(19) United States
(12) Patent Application Publication
(10) Pub. No.: US 2016/0166281 A1
(43) Pub. Date: Jun. 16, 2016

(54) INSERTION TOOL AND INSERTION METHOD FOR INSERTION TOOL

(71) Applicant: TERUMO KABUSHIKI KAISHA, Tokyo (JP)

(72) Inventors: Masakatsu KAWAURA, Sunnyvale, CA (US); Nao MANDAI, Mountain View, CA (US)

(73) Assignee: TERUMO KABUSHIKI KAISHA, Tokyo (JP)

(21) Appl. No.: 15/051,094

(22) Filed: Feb. 23, 2016

Related U.S. Application Data
(63) Continuation of application No. PCT/JP2014/074253, filed on Sep. 12, 2014.

(30) Foreign Application Priority Data
Sep. 20, 2013 (JP) 2013-196165

(51) Int. Cl. A61B 17/34; A61F 2/00

(52) U.S. Cl. CPC A61B 17/3468; A61F 2/0045; A61F 2002/0072; A61B 2017/3492

(57) ABSTRACT

An insertion tool is disclosed, which includes a vaginal-insertion tool having a vaginal-insertion portion configured to be inserted into a vagina and a suction portion provided at the vaginal-insertion portion; a urethral-insertion tool having a urethral-insertion portion configured to be inserted into a urethra; a maintaining portion capable of maintaining a relative positional relationship between the urethral-insertion portion and the vaginal-insertion portion; and a displacement portion capable of causing a distal portion of the vaginal-insertion portion to approach the urethral-insertion portion rather than the relative positional relationship maintained by the maintaining portion.

### CROSS-REFERENCES TO RELATED APPLICATIONS

This application is a continuation of International Application No. PCT/JP2014/074253 filed on Sep. 12, 2014, and claims priority to Japanese Application No. 2013-196165 filed on Sep. 20, 2013, the entire content of both of which is incorporated herein by reference.

### TECHNICAL FIELD

The present disclosure generally relates to an insertion tool and an insertion method for an insertion tool.

### BACKGROUND DISCUSSION

If a person suffers from a urinary incontinence, specifically, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth or the like.

For the treatment of urinary incontinence, a surgical treatment can be effective, in which there is used, for example, a belt-shaped living body tissue supporting indwelling article called “sling.” The sling is indwelled inside the body and the urethra is supported by the sling (for example, refer to Japanese Patent Laid-Open No. 2010-99499). In order to indwell the sling inside the body, an operator would incise the vagina with a surgical knife, dissect the part between the urethra and vagina, and make the dissected region and the outside communicate with each other through obturator foramens by use of a puncture needle or the like. Then, in this state, the sling is indwelled into the body.

If the vagina is incised once, however, a situation may occur that the sling is exposed to the inside of the vagina from a wound caused by the incision, and complications may be caused by an infection from the wound. Further, since the vagina is incised, the burden on the patient can be relatively heavy. Further, the urethra or the like may be damaged in the course of the procedure by the operator. In addition, the fingertip of the operator himself/herself may be damaged.

### SUMMARY

It is desirable to provide an insertion tool and an insertion method for an insertion tool wherein a wide space can be secured between the urethral wall and the vaginal wall while the burden on a living body can be reduced.

An insertion tool is disclosed, which includes a vaginal-insertion tool having a vaginal-insertion portion configured to be inserted into a vagina and a suction portion provided at the vaginal-insertion portion; a urethral-insertion tool having a urethral-insertion portion configured to be inserted into a urethra; a maintaining portion capable of maintaining a relative positional relationship between the urethral-insertion portion and the vaginal-insertion portion; and a displacement portion capable of causing a distal portion of the vaginal-insertion portion to approach the urethral-insertion portion rather than the relative positional relationship maintained by the maintaining portion.

In accordance with an exemplary embodiment, the insertion tool is configured such that the suction portion is provided at least in the vaginal-insertion portion on a side of the urethral-insertion portion.

In accordance with an exemplary embodiment, the insertion tool is configured such that the vaginal-insertion tool has a vaginal-insertion portion supporting portion supporting the vaginal-insertion portion, the urethral-insertion tool has a urethral-insertion portion supporting portion supporting the urethral-insertion portion, and the displacement portion interlocks the vaginal-insertion portion supporting portion and the urethral-insertion portion supporting portion for rotary movement to each other.

Alternatively, the insertion tool may be configured such that the vaginal-insertion tool has a vaginal-insertion portion supporting portion supporting the vaginal-insertion portion, the urethral-insertion tool has a urethral-insertion portion supporting portion supporting the urethral-insertion portion, and the displacement portion interlocks the vaginal-insertion portion supporting portion and the urethral-insertion portion supporting portion for movement toward and away from each other.

In accordance with an exemplary embodiment, the maintaining portion may include a pawl portion provided at one of the vaginal-insertion portion supporting portion and the urethral-insertion portion supporting portion and an engagement portion provided at the other one of the vaginal-insertion portion supporting portion and the urethral-insertion portion supporting portion and configured to engage with the pawl portion.

In accordance with an exemplary embodiment, the engagement between the pawl portion and the engagement portion can be cancelled.

In accordance with an exemplary embodiment, the displacement portion may include a hinge portion provided at an intermediate portion in a longitudinal direction of the vaginal-insertion portion for allowing the vaginal-insertion portion to be flexion-deformed at the intermediate portion in the longitudinal direction.

In accordance with an exemplary embodiment, the maintaining portion may include an operation portion disposed on an outer periphery of the vaginal-insertion portion and movable in the longitudinal direction with respect to the vaginal-insertion portion, and the maintaining portion blocks the flexion-deformation by positioning the operation portion so as to overlap with the hinge portion but allows the flexion-deformation by positioning the operation portion so as to displace from the hinge portion.

According to another aspect of the present disclosure, an insertion method is disclosed for an insertion tool having a vaginal-insertion tool including a vaginal-insertion portion configured to be inserted into a vagina and having a suction hole provided at the vaginal-insertion portion and a urethral-insertion tool including a urethral-insertion portion configured to be inserted into a urethra, the method including: inserting the vaginal-insertion portion into the vagina and inserting the urethral-insertion portion into the urethra; displacing at least one of the vaginal-insertion portion and the urethral-insertion portion in a direction spaced away from the other one of the vaginal-insertion portion and the urethral-insertion portion in a state in which a vaginal wall is sucked by the suction hole; and maintaining a relative positional relationship between the vaginal-insertion portion and the urethral-insertion portion after the displacement.

With the present disclosure, since the distal portion of the vaginal-insertion portion can be moved toward the urethral-insertion portion by the displacement portion, absorption of the vaginal wall by the vaginal-insertion portion can be performed with a relatively higher degree of certainty and a sufficient space can be secured between the vagina and the urethra. In particular, by inserting the vaginal-insertion portion and the urethral-insertion portion into the vagina and the urethra, respectively, and moving the vaginal-insertion portion toward the urethral-insertion portion, the vaginal-insertion portion can be closely contacted with the vaginal wall. In this state, the vaginal wall can be absorbed with a higher degree of certainty to the vaginal-insertion portion. Then, after the vaginal wall is absorbed, the vagina can be separated sufficiently from the urethra by spacing the vaginal-insertion portion away from the urethra insertion portion and maintaining (fixing) the postures of them by the maintaining portion. Consequently, a space sufficient for puncture by the puncture member can be secured between the vagina and the urethra. Therefore, puncture by the puncture member can be performed with relative safety.

### BRIEF DESCRIPTION OF THE DRAWINGS

FIG. 1 is a perspective view depicting an example of an implant;

FIG. 2 is a perspective view depicting a puncture apparatus according to a first embodiment;

FIG. 3 is a lateral view of the puncture apparatus depicted in FIG. 2;

FIG. 4 is a plan view depicting an operation member, which the puncture apparatus as depicted in FIG. 2 includes;

FIGS. 5A and 5B depict a puncture member which the puncture apparatus depicted in FIG. 2 includes, wherein FIG. 5A is a perspective view and FIG. 5B is a sectional view taken along line VB-VB in FIG. 5A;

FIG. 6 is a cross sectional view of the puncture member depicted in FIGS. 5A and 5B;

FIGS. 7A to 7C depict a state maintaining portion, which the puncture member depicted in FIGS. 5A and 5B includes, wherein FIG. 7A is a top plan view and FIGS. 7B and 7C are sectional views;

FIGS. 8A to 8C are partial enlarged views depicting the state maintaining portion which the puncture member depicted in FIGS. 5A and 5B has, wherein FIGS. 8A and 8B are plan views of modifications and FIG. 8C is a plan view of the present embodiment;

FIG. 9 is a plan view depicting a guide portion of a frame provided in the puncture apparatus as depicted in FIG. 2;

FIG. 10 is a sectional view of the guide portion depicted in FIG. 9;

FIG. 11 is a plan view depicting a fixing portion of the frame, which the puncture apparatus depicted in FIG. 2 includes;

FIG. 12 is a lateral view of an insertion tool, which the puncture apparatus as depicted in FIG. 2 includes;

FIGS. 13A and 13B are sectional views illustrating a function of a marker provided at a urethral-insertion portion in an inappropriate case and an appropriate case, respectively;

FIG. 14 is a sectional view illustrating the function of the marker provided at the urethral-insertion portion;

FIGS. 15A and 15B are a lateral view and a front view, respectively, depicting a positional relationship between the puncture member and the obturator foramen (pelvis);

FIGS. 16A and 16B are partial enlarged views of a vaginal-insertion tool, which the insertion tool as depicted in FIG. 12 includes, wherein FIG. 16A is a top plan view and FIG. 16B is a sectional view;

FIG. 17A is a sectional view depicting an example of a shape of a vaginal wall, and FIG. 17B is a sectional view depicting a vaginal-insertion portion in a state in which it is inserted in a vagina depicted in FIG. 17A;

FIG. 18 is a sectional view depicting the vaginal-insertion portion depicted in FIG. 12 in a state in which it is inserted in the vagina;

FIGS. 19A and 19B illustrate a maintaining portion and a displacement portion of the insertion tool depicted in FIG. 12, wherein FIG. 19A is a view depicting a locked state and FIG. 19B is a view depicting an unlocked state;

FIGS. 20A and 20B are views illustrating an operation procedure of the puncture apparatus depicted in FIG. 2;

FIGS. 21A and 21B are views illustrating another operation procedure of the puncture apparatus depicted in FIG. 2;

FIGS. 22A and 22B are views illustrating still another operation procedure of the puncture apparatus depicted in FIG. 2;

FIG. 23 is a lateral view illustrating a relationship between the puncture apparatus and the pelvis in a state illustrated in FIG. 22B;

FIG. 24 is a view illustrating an operation procedure of the puncture apparatus depicted in FIG. 2;

FIGS. 25A and 25B are views illustrating the operation procedure of the puncture apparatus depicted in FIG. 2;

FIG. 26 is a lateral view illustrating a relationship between the puncture apparatus and the pelvis in a state illustrated in FIG. 25A;

FIG. 27 is a sectional view depicting a posture of the puncture member with respect to a urethra in a state illustrated in FIG. 25B;

FIGS. 28A and 28B are views illustrating the operation procedure of the puncture apparatus depicted in FIG. 2;

FIG. 29 is a view illustrating the operation procedure of the puncture apparatus depicted in FIG. 2;

FIG. 30 is a lateral view depicting an insertion tool, which a puncture apparatus according to a second embodiment includes;

FIG. 31 is a lateral view depicting an insertion tool, which a puncture apparatus according to a third embodiment includes;

FIG. 32 is a lateral view depicting an insertion tool, which a puncture apparatus according to a fourth embodiment includes;

FIG. 33 is a sectional view depicting a urethral-insertion tool, which shows the insertion tool depicted in FIG. 32 in a state in which it is inserted in a urethra;

FIGS. 34A and 34B are lateral views depicting an insertion tool, which a puncture apparatus according to a fifth embodiment includes, wherein FIG. 34A is a view depicting the insertion tool in a state in which it is locked by a maintaining portion and FIG. 34B is a view depicting the insertion tool in another state in which it is not locked by the maintaining portion;

FIG. 35A is a lateral view of an insertion tool, which a puncture apparatus according to a sixth embodiment includes;

FIG. 35B is a lateral view depicting the insertion tool in a state in which it is locked by a maintaining portion;

FIG. 35C is a lateral view depicting the insertion tool in another state in which it is not locked by the maintaining portion;

FIG. 36 is a lateral view of an insertion tool, which a puncture apparatus according to a seventh embodiment includes;

FIG. 37 is a sectional view depicting a vaginal-insertion tool depicted in FIG. 36 in a state in which it is inserted in a vagina;

FIG. 38 is a lateral view of an insertion tool, which a puncture apparatus according to an eighth embodiment includes;

FIGS. 39A and 39B depict a distal portion of a vaginal-insertion tool depicted in FIG. 38, wherein FIG. 39A is a top plan view and FIG. 39B is a sectional view taken along line XXXIXB-XXXIXB in FIG. 39A;

FIGS. 40A and 40B depict the distal portion depicted in FIGS. 39A and 39B in a state in which it is inserted in a vagina, wherein FIG. 40A is a view depicting the distal portion in a state before a vaginal wall is absorbed and FIG. 40B is a view depicting the distal portion in another state in which the vaginal wall is absorbed;

FIG. 41 is a sectional view depicting a distal portion of a vaginal-insertion tool, which a puncture apparatus according to a ninth embodiment includes;

FIGS. 42A and 42B depict the distal portion depicted in FIG. 41 in a state in which it is inserted in a vagina, wherein FIG. 42A is a view depicting the distal portion before a vaginal wall is absorbed and FIG. 42B is a view depicting the distal portion in a state in which the vaginal wall is absorbed;

FIG. 43 is a sectional view depicting a distal portion of a vaginal-insertion tool, which a puncture apparatus according to a tenth embodiment includes;

FIGS. 44A to 44C depict a distal portion of a vaginal-insertion tool, which a puncture apparatus according to an eleventh embodiment includes, wherein FIG. 44A is a lateral view, FIG. 44B is a top plan view, and FIG. 44C is a bottom plan view;

FIGS. 45A and 45B depict the distal portion depicted in FIGS. 44A to 44C in a state in which it is inserted in a vagina, wherein FIG. 45A is a view depicting the distal portion in a state before a vaginal wall is absorbed and FIG. 45B is a view depicting the distal portion in another state in which the vaginal wall is absorbed;

FIG. 46 is a view depicting a distal portion of a vaginal-insertion tool, which a puncture apparatus according to a twelfth embodiment includes;

FIGS. 47A and 47B depict a distal portion of a vaginal-insertion tool, which a puncture apparatus according to a thirteenth embodiment includes, wherein FIG. 47A is a lateral view and FIG. 47B is a sectional view;

FIGS. 48A and 48B are views illustrating a function of the distal portion depicted in FIGS. 47A and 47B;

FIG. 49 is a lateral view depicting a urethral-insertion tool, which a puncture apparatus according to a fourteenth embodiment includes;

FIG. 50 is a lateral view depicting a urethral-insertion tool, which a puncture apparatus according to a fifteenth embodiment includes;

FIGS. 51A and 51B are partial sectional views depicting a urethral-insertion portion, which a puncture apparatus according to a sixteenth embodiment includes;

FIG. 52 is a perspective view depicting a puncture member, which a puncture apparatus according to a seventeenth embodiment includes; and

FIG. 53 is a sectional view depicting a modification to the puncture member depicted in FIG. 52.

### DETAILED DESCRIPTION

In the following, a puncture apparatus to which an insertion tool of the present disclosure is applied and an insertion method for the insertion tool are described in detail in connection with preferred embodiments depicted in the accompanying drawings.

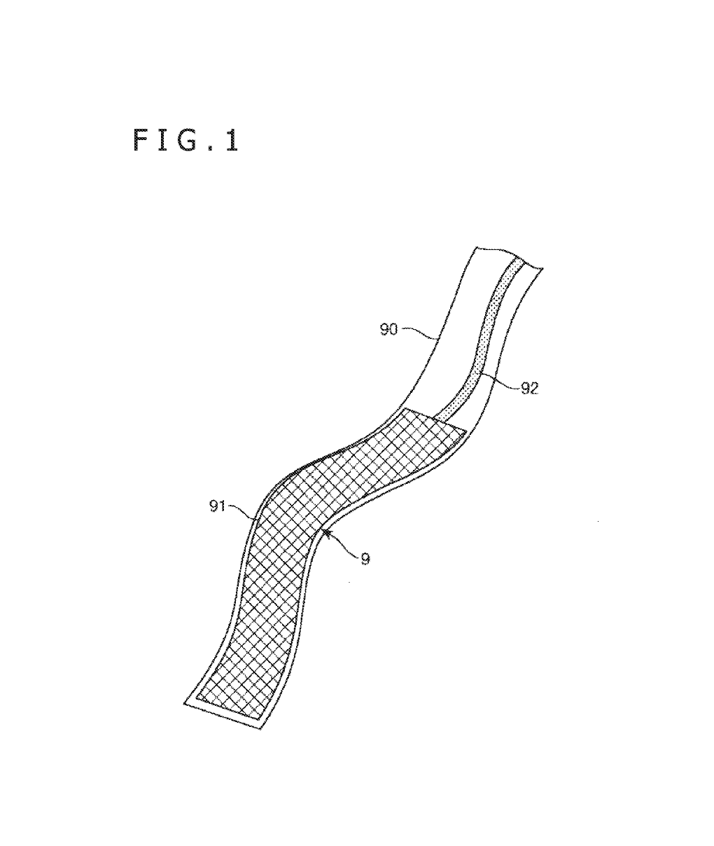

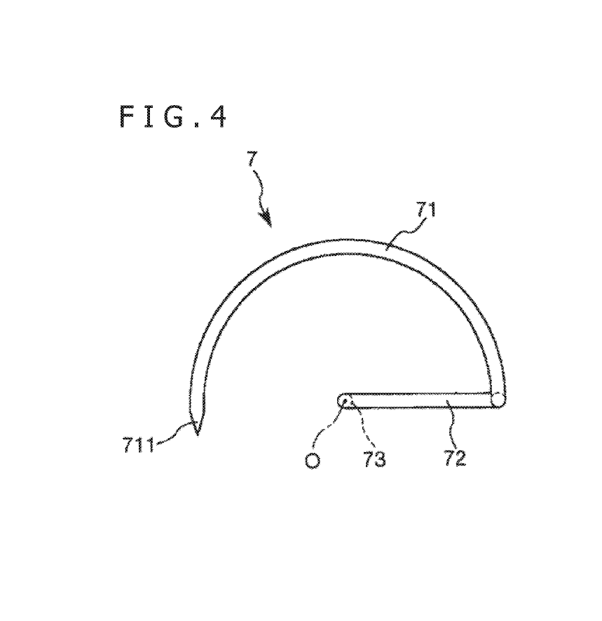

First, a puncture apparatus according to a first embodiment is described below with reference to FIGS. 1 to 29.

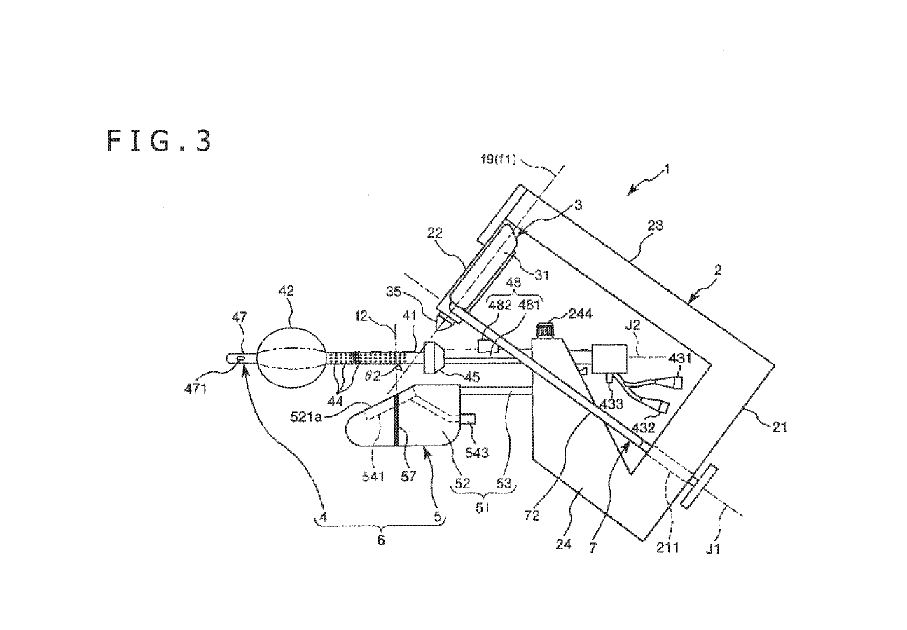

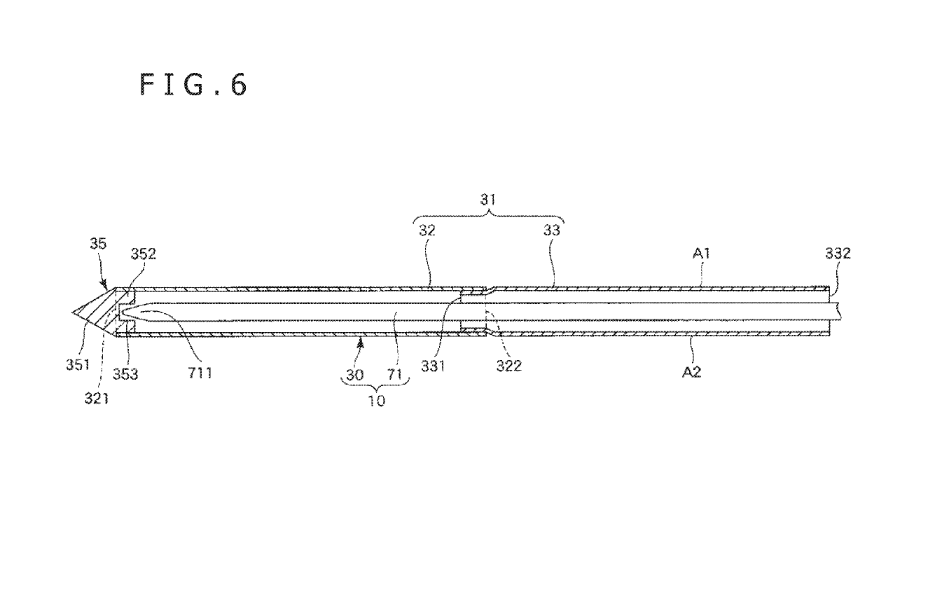

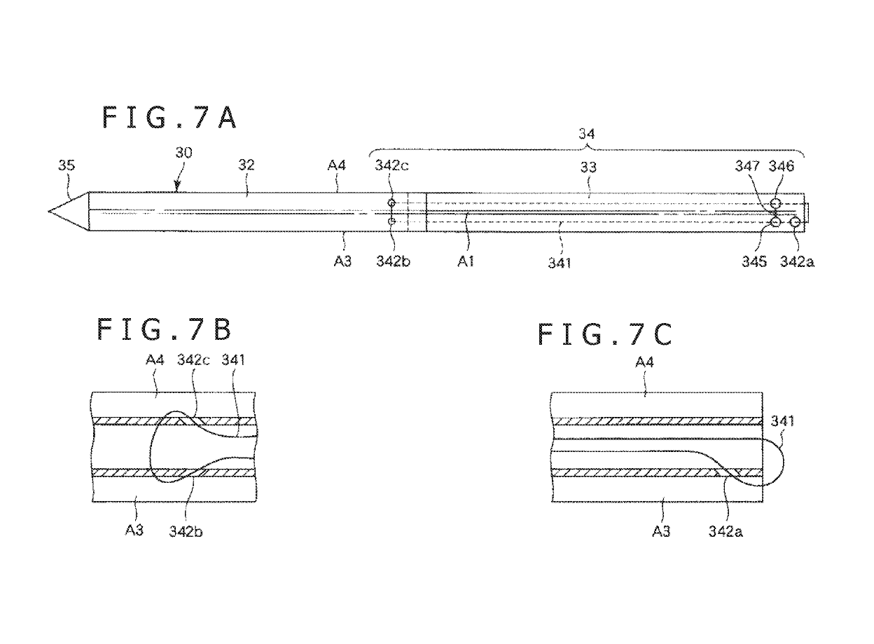

Note that, in the following description, the left side in FIG. 3 is referred to as “distal end,” the right side as “proximal end,” the upper side as “upper,” and the lower side as “lower” for the convenience of description. FIG. 3 depicts the puncture apparatus in a state in which it has not been used, and in the following description, this state is referred to as “initial state” for the convenience of description. Another state in which the puncture apparatus (insertion tool) depicted in FIG. 3 is mounted on a patient is referred to also as “mounted state” for the convenience of description. Furthermore, in FIGS. 6, 7A, 7B, and 7C, the puncture member, which extends in an arc is depicted in a linearly stretched state for the convenience of illustration.

First, an example of an implant (living body tissue supporting indwelling article) 9 for being embedded in a living body by the puncture apparatus is described.

The implant 9 depicted in FIG. 1 is a tool, which can be embedded in a living body for the treatment of female urinary incontinence. More particularly, the implant 9 is a tool for supporting a urethra, for example, a tool for supporting, when the urethra tends to move to the vaginal wall side, the urethra so as to restrict its movement in a direction in which the urethra is spaced away from the vaginal wall. As such, an implant 9 as just described, for example, an elongated article having flexibility can be used.

As depicted in FIG. 1, the implant 9 has an implant main body 91 and a belt 92 interlocked to one end of the implant main body 91. Note that, for example, a guide wire, a cord, a string, or the like may be used in place of the belt 92. The implant main body 91 has a form of a net and has a belt-like general shape. The implant main body 91 can be configured from an article formed by crossing linear objects with each other into a braid, namely, for example, a net-like braid. The linear objects may be those having a circular transverse sectional shape, or those having a flattened transverse sectional shape, namely, those of strip-like shape or the like.

The implant 9 having such a configuration as described above is accommodated in a packaging material 90 which is sterilized in an unused state. Consequently, contamination of the implant 9 can be prevented.

The constituent material of each of the implant main body 91, belt 92 and packaging material 90 is not restricted particularly, and various resin materials having biocompatibility such as polypropylene, polyester, nylon, etc. and their fibers can be used.

Although the implant 9 is described above, the implant 9 is not limited to the aforementioned net-like one only if it can demonstrate similar effects.

Further, a “pelvic treatment kit” can be configured from such an implant 9 as described above and a sheath 30 hereinafter described.

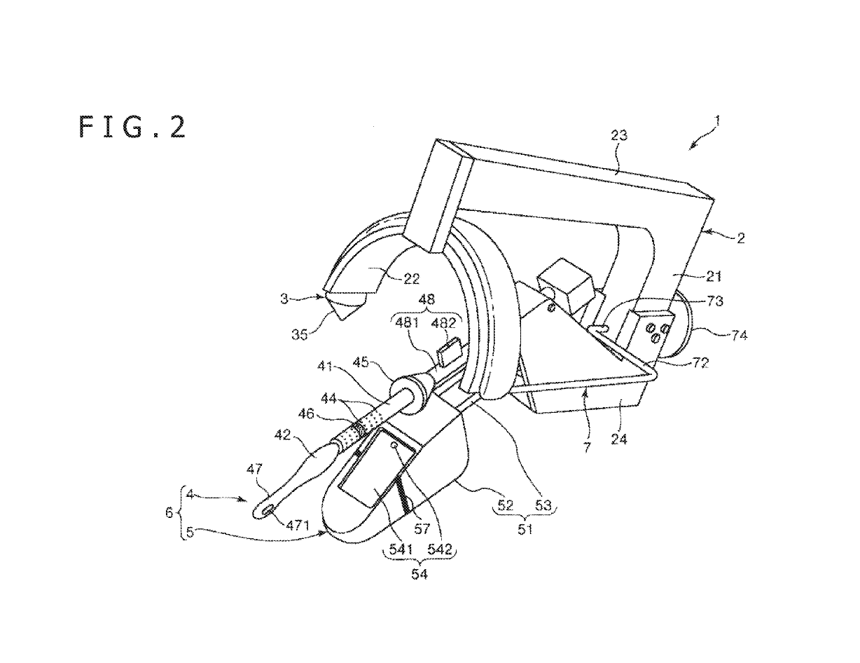

A puncture apparatus 1 depicted in FIG. 2 is an apparatus for embedding the aforementioned implant 9 into a living body.

As depicted in FIG. 2, the puncture apparatus 1 can include a frame (supporting portion) 2, an insertion tool 6, an operation member 7, and a puncture member 3. The puncture member 3, the insertion tool 6, and the operation member 7 are supported on the frame 2, and the puncture member 3 is supported on the operation member 7. Further, the insertion tool 6 has a urethral-insertion tool 4 and a vaginal-insertion tool 5.

In the following, the components mentioned are described in order.

The operation member 7 is a member for supporting and operating the puncture member 3. As shown in FIGS. 2, 3, and 4, such an operation member 7 as just described can include an insertion portion 71, a shaft portion 73, and an interlock portion 72, which interlocks the insertion portion 71 and the shaft portion 73 to each other. The insertion portion 71, interlock portion 72, and shaft portion 73 may be formed integrally with one another, or, alternatively, at least one of them may be formed as a separate body from the others of them.

The insertion portion 71 is a portion to be inserted into the puncture member 3 and functions as a stylet for reinforcing the puncture member 3 from the inner side. By inserting the insertion portion 71 into the puncture member 3, the puncture member 3 is connected to the operation member 7, whereby an operation of the puncture member 3 by the operation member 7 (puncture of a living body) is permitted.

The insertion portion 71 has an arcuate shape corresponding to the shape of the puncture member 3. The center angle of the insertion portion 71 is set in accordance with the center angle of the puncture member 3. In accordance with an exemplary embodiment, a distal portion 711 of the insertion portion 71 is tapering, by which the insertion of the insertion portion 71 into the puncture member 3 can be performed relatively smoothly.

Note that, while the transverse sectional shape of the insertion portion 71 in the present embodiment is a circular shape, the transverse sectional shape of the insertion portion 71 is not restricted to this, but may be, for example, an elliptic shape, a diamond shape rounded at the corners thereof, a rectangular shape (flat shape, plate shape) rounded at the corners thereof, or a spindle shape having an increased width (increased dimension) at a central portion with respect to the opposite end portions thereof. By applying such transverse sectional shapes, in a state in which the insertion portion 71 is inserted in the puncture member 3, rotation and so forth of the puncture member 3 with respect to the insertion portion 71 can be reduced relatively effectively.

The shaft portion 73 extends along an axis J1, which intersects with the center O of the insertion portion 71 and is orthogonal to a plane f1, which can include the insertion portion 71. In accordance with an exemplary embodiment, the interlock portion 72 interlocks a proximal portion of the insertion portion 71 and a distal portion of the shaft portion 73. The interlock portion 72 has a substantially L shape bent at a substantially right angle at an intermediate portion thereof.

The operation member 7 having such a configuration as described above is configured so as to have a higher rigidity than that of the puncture member 3. The material constituting the operation member 7 is not restricted particularly and, for example, various metallic materials such as stainless steel, aluminum or aluminum alloys, titanium or titanium alloys and so forth can be used. Note that, in order to reduce distortion and so forth of the puncture member 3, the interlock portion 72 and the shaft portion 73 may be formed thicker than the insertion portion 71 as occasion demands.

Further, a handle 74 can be fixed to a proximal portion of the shaft portion 73 such that the operation member 7 can be operated to turn around the axis J1 by rotationally operating the handle 74. Note that, for example, the interlock portion 72 may be grasped to operate the operation member 7 to rotate while the handle 74 is omitted.

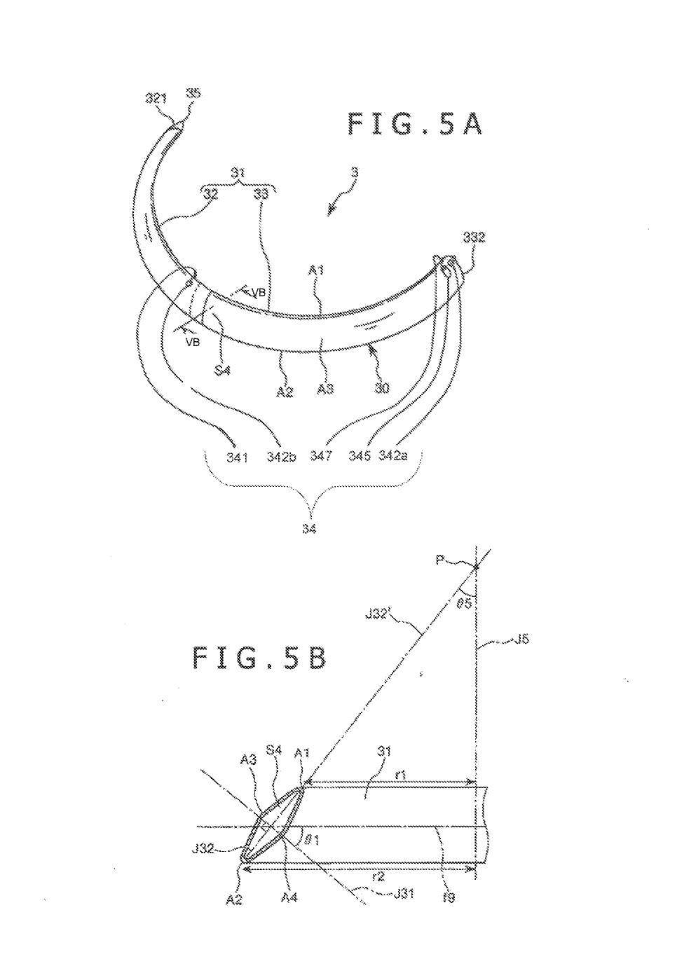

The puncture member 3 is a member for puncturing a living body. As shown in FIG. 5A, the puncture member 3 can include an elongated sheath (medical tube) 30, and a needle body 35 provided at the distal end of the sheath 30. The sheath 30 can include a tubular main body 31, and a state maintaining portion 34.

The main body 31 is configured from an elongated pipe member (tube) and is open at the distal end and the proximal end thereof. The inner side of the main body 31 can function as a space into which the insertion portion 71 is to be inserted and into which the implant 9 is to be inserted.

The main body 31 has a curved shape in which it is curved in an arc. Further, the main body 31 has a flattened transverse sectional shape having a minor axis J31 and a major axis J32 as depicted in FIG. 5B. By forming the main body 31 in a flattened shape, the implant main body 91 can be controlled to a desired posture in the main body 31.

Note that the flattened shape of the main body 31 is not restricted particularly and can be, for example, an elliptical shape, a sectional shape of a convex lens, a diamond shape rounded at the corners thereof, a rectangle (flattened shape) rounded at the corners thereof or a spindle shape having a width greater (diameter increased) at a central portion than at the opposite end portions thereof.

Meanwhile, the width (length in the major axis J32 direction) of the internal space of the main body 31 is designed so as to be substantially equal to the width of the implant main body 91. By the configuration, the frictional resistance between the implant 9 and the main body 31 can be reduced, such that unnecessary force is not applied to the implant 9, and the implant main body 91 can be disposed into the main body 31 in a state in which it is developed sufficiently. However, the width of the internal space of the main body 31 may be smaller than the width of the implant main body 91. In this case, the width of the main body 31 can be shorten, and therefore, the puncture member 3 becomes minimally invasive.

Note that, in the following description, for the convenience of description, an end portion positioned on the inner side in the major axis J32 direction is referred to also as “inner circumferential portion A1,” an end portion positioned on the outer side in the major axis J32 direction be referred to also as “outer circumferential portion A2,” a face directed to the upper side is referred to also as “front face A3,” and a face directed to the lower side is referred to also as “back face A4,” as depicted in FIG. 5B.

Where, as depicted in FIG. 5B, a plane including the center point of the arc of a central portion S4 and the center point of a transverse sectional shape across the longitudinal direction of the main body 31 (plane including the center axis of the main body 31) is represented as plane f9 and the angle between the plane f9 and the minor axis J31 at the central portion S4 is represented as inclination angle θ1, the inclination angle θ1 preferably is an acute angle. Where the inclination angle θ1 is an acute angle, the implant main body 91 can be disposed substantially in parallel to the urethra as hereinafter described. Therefore, the urethra can be supported relatively effectively.

Although the inclination angle θ1 is not restricted particularly only if it is an acute angle, for example, the inclination angle θ1, for example, preferably is approximately 20 degrees to 60 degrees, more preferably is 30 degrees to 45 degrees, and most preferably is 35 degrees to 40 degrees. The aforementioned effects are further enhanced thereby.

Note that, although it is preferable for the inclination angle θ1 to satisfy the aforementioned numerical range over the overall region in the extending direction of the main body 31, only it is necessary for the inclination angle θ1 to satisfy the aforementioned numerical range at least at the central portion S4. Here, the “central portion S4” signifies a region including a region positioned between the urethra and the vagina at least in a state in which a living body is punctured by the puncture member 3 (a state in which the main body 31 is disposed inside the living body).

A marker may be provided at portions, which protrude to the outside of a living body in a state in which the main body 31 is disposed in the living body, of the opposite end portions of the main body 31 at positions spaced by an equal distance from the central portion S4, which makes it possible to confirm the position of the central portion S4 inside the living body by comparing the positions of both markers.

The configuration of the main body 31 can be described in the following manner. In particular, the main body 31 is formed such that the major axis J32 is inclined with respect to a center axis J5 of the arc and the center axis J5 and an extension line J32′ of the major axis J32 have an intersection P as depicted in FIG. 5B. In this case, the angle θ5 defined between the center axis J5 and the extension line J32′ is equal to the inclination angle θ1.

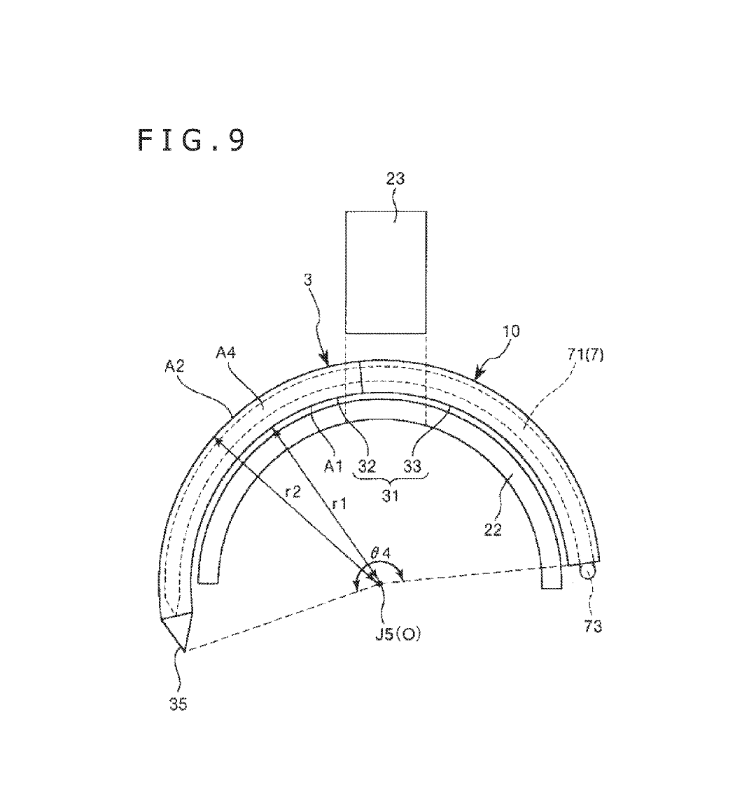

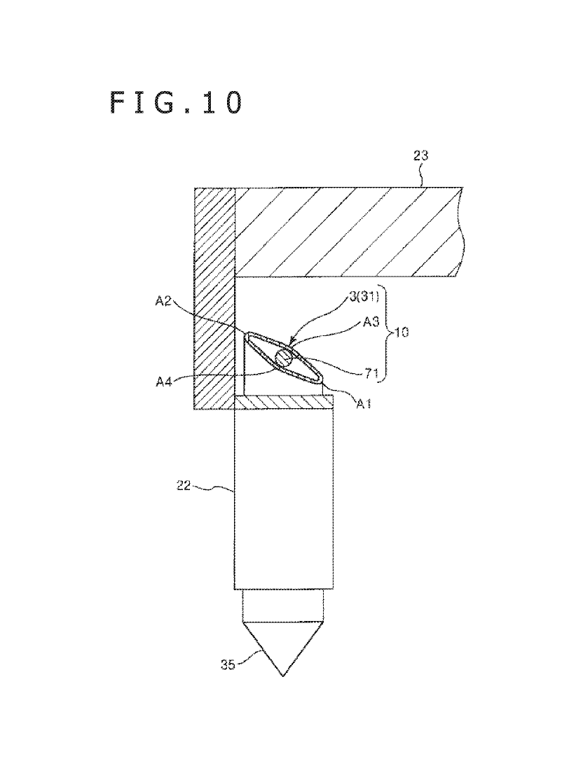

Alternatively, for example, also it can be regarded that, in a plan view as viewed in the direction of the center axis J5 of the main body 31 as depicted in FIG. 9, the main body 31 has the inner circumferential portion A1 positioned on an inner circumferential edge thereof and having a minimum radius of curvature r1 and the outer circumferential portion A2 positioned on an outer circumferential edge thereof and having a maximum radius of curvature r2, and, as depicted in FIGS. 5B and 10, the inner circumferential portion A1 and the outer circumferential portion A2 are positioned in a displaced relationship from each other in the direction of the center axis J5.

Such a main body 31 as described above is configured such that two divisional pieces are connected to each other such that the main body 31 can be divided halfway. In particular, for example, the main body 31 has a distal end divisional piece 32 positioned on the needle body 35 side and a proximal end divisional piece 33 positioned on the proximal side of the distal end divisional piece 32. The distal end divisional piece 32 and the proximal end divisional piece 33 are connected for separation from each other. Further, in the main body 31, the distal end divisional piece 32 and the proximal end divisional piece 33 have lengths substantially equal to each other, and the boundary between them is positioned at the central portion S4.

As depicted in FIG. 6, the distal end divisional piece 32 has a tubular shape and has a distal side opening 321 and a proximal side opening 322. In accordance with an exemplary embodiment, the proximal end divisional piece 33 has a tubular shape and has a distal side opening 331 and a proximal side opening 332. A distal portion of the proximal end divisional piece 33 is inserted in a proximal portion of the distal end divisional piece 32, whereby the distal end divisional piece 32 and the proximal end divisional piece 33 are connected to each other. By inserting the proximal end divisional piece 33 in the distal end divisional piece 32, a step which may possibly appear on the boundary between the divisional pieces 32 and 33 is less likely to be caught by the living body tissue at the time of puncture by the puncture member 3. Consequently, puncture of a living body by the puncture member 3 can be performed relatively smoothly. However, conversely to the present embodiment, a proximal portion of the distal end divisional piece 32 may be inserted in the proximal end divisional piece 33 to thereby connect the divisional pieces 32 and 33 together.

The state (connected state) in which the divisional pieces 32 and 33 are connected together is maintained by the state maintaining portion 34. As depicted in FIG. 7A, the state maintaining portion 34 has holes 342a, 342b and 342c, an endless string 341 fitted in the holes 342a, 342b and 342c, exposure holes 345 and 346 for exposing the string 341, and a slit 347 interconnecting the exposure holes 345 and 346.

In accordance with an exemplary embodiment, the hole 342a can be provided at a proximal portion of the proximal end divisional piece 33 rather near to the inner circumferential portion A1 of the front face A3. In accordance with an exemplary embodiment, the holes 342b and 342c can be provided in an opposing relationship to each other at positions of the front face A3 and the back face A4 of a proximal portion of the distal end divisional piece 32 rather near to the inner circumferential portion A1.

The string 341 is disposed in the main body 31 and is routed outside the main body 31 between the hole 342b and the hole 342c and between the hole 342a and the proximal side opening 332. Consequently, the connection state of the divisional pieces 32 and 33 can be simply maintained. Further, exposure of the string 341 to the outside of the main body 31 can be reduced, and the string 341 becomes less likely to be caught by a living body tissue. In accordance with an exemplary embodiment, the overall length of the string 341 can be made as short as possible, therefore, the string 341 is less likely to be caught by the implant 9 at the time of inserting and passing the implant 9 into and through the main body 31. Especially, for example, since the holes 342a, 342b and 342c are disposed rather near to the inner circumferential portion A1 as described hereinabove, also the string 341 is disposed biasedly rather near to the inner circumferential portion A1. Therefore, the string 341 becomes less likely to be caught by the implant 9.

The string 341 can be obtained, for example, by preparing a string having ends, inserting one end of the string into the main body 31 via the proximal side opening 332, drawing out the same to the outside of the main body 31 through the hole 342b, inserting the same into the main body 31 via the hole 342c, drawing out the same to the outside of the main body 31 through the hole 342a and tying the same with the other end of the string in the proximity of the proximal side opening 332. Note, however, that the position of the knot is not limited.

Here, as shown in FIG. 7C, the axis of the hole 342a is inclined such that the outer side opening is positioned on the proximal side with respect to the inner side opening. In accordance with an exemplary embodiment, as shown in FIG. 7B, the axis of each of the holes 342b and 342c is inclined such that the outer side opening is positioned on the distal side with respect to the inner side opening, which makes it possible to allow each of the holes 342a, 342b and 342c to extend along the route of the string 341. Consequently, the string 341 is less likely to be caught by the holes 342a, 342b and 342c.

Note that the number and the disposition of holes through which the string 341 passes are not restricted particularly only if the connected state of the distal end divisional piece 32 and the proximal end divisional piece 33 can be maintained by the string 341. Further, the string 341 need not be in an endless state but may be in an ended state in which it has one end and the other end. For example, an ended string may be prepared and passed at one end thereof through the hole 342a and the proximal side opening 332 to form a loop while it passes at the other end thereof through the holes 342b and 342c to form a loop. Further, for example, the string 341 can include a cord or a belt, which can be used similarly to the string 341.

In accordance with an exemplary embodiment, the exposure holes 345 and 346 can be provided in an opposing relationship to each other at the front face A3 and the back face A4 of the proximal portion of the proximal end divisional piece 33. Further, the exposure holes 345 and 346 can be provided at locations at which they are exposed from the body surface when the main body 31 is disposed in a living body. Further, the exposure holes 345 and 346 can be positioned on the route of the string 341 and the string 341 is exposed through the exposure holes 345 and 346. Further, the exposure holes 345 and 346 can be tied with each other by the slit 347 provided in the inner circumferential portion A1 along a circumferential direction of the main body 31.

In such a state maintaining portion 34 as described above, if the string 341 is cut, then a state in which the distal end divisional piece 32 and the proximal end divisional piece 33 are separable from each other can be established. By the configuration, the distal end divisional piece 32 and the proximal end divisional piece 33 can be placed into a separable state through a simple operation. Further, since the cutting of the string 341 is visually observable, it can be confirmed that the distal end divisional piece 32 and the proximal end divisional piece 33 are in a separable state.

In accordance with an exemplary embodiment, by providing the exposure holes 345 and 346 and the slit 347 in such a manner as in the present embodiment, the string 341 can be simply cut. Describing by way of example, scissors are prepared, and one of blades thereof is fitted into the exposure holes 345 and 346 and the string 341 is positioned between the one and the other blades. Then, if the scissors are operated to close, then at least one of the pair of blades passes through the slit 347 until the pair of blades are overlapped with each other, and the string 341 is cut during the process.

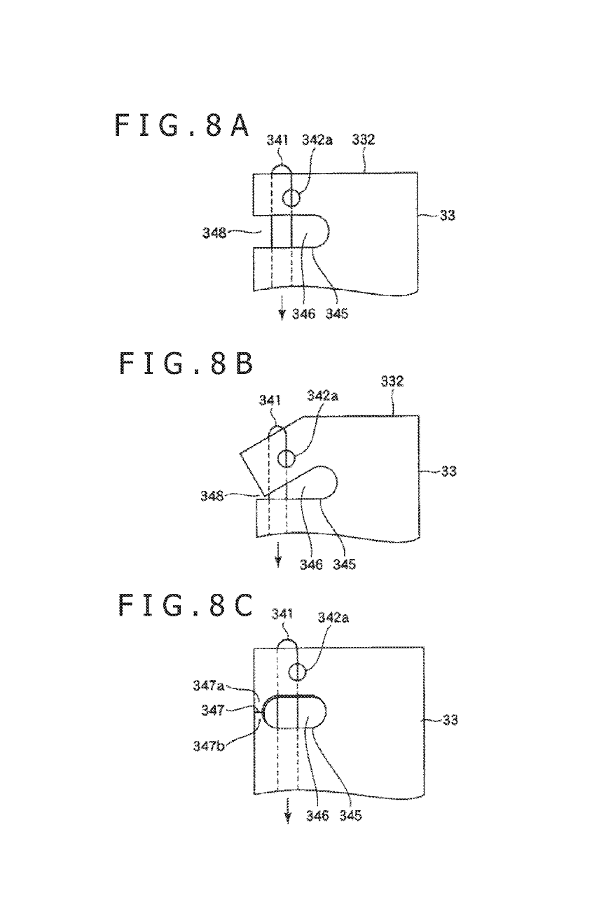

In this manner, in this embodiment, the slit 347 is used as a passing route of a blade. By this, cutting of the string 341 can be performed as described above, and deformation of the main body 31 by the tension on the string 341 can be prevented. For example, the passing route of a blade may be configured from a hole 348 in place of the slit 347 as shown in FIG. 8A. In this case, however, depending upon the hardness of the main body 31 or the like, the hole 348 may be crushed through buckling by the tension on the string 341 as depicted in FIG. 8B, resulting in deformation of the main body 31. In contrast, in the case of the slit 347, since portions 347a and 347b on the opposite sides of the slit 347 attach and are pressed against each other as depicted in FIG. 8C, such a deformation as described above will not occur. Consequently, the main body 31 can be prevented from deformation.

The main body 31 described above has the needle body 35 at the distal end thereof. As depicted in FIG. 6, the needle body 35 can include a tapering needle tip 351, and a proximal portion 352 provided on the proximal side of the needle tip 351. The proximal portion 352 is inserted in the main body 31, whereby the needle body 35 is held on the main body 31 in a freely detachable manner. The proximal portion 352 is fitted in the main body 31 with force of such a degree that the needle body 35 can be prevented from being unintentionally removed from the main body 31.

Note that the needle body 35 may be configured integrally with the main body 31, and in this case, only it is necessary for the needle body 35 to be cut away from the main body 31 by scissors or the like.

Further, an engaging portion 353 for engaging with the distal portion 711 of the insertion portion 71 is provided at the proximal portion 352. The engaging portion 353 is configured from a recess, and in an insertion state in which the insertion portion 71 is inserted in the puncture member 3, the distal portion 711 is positioned in the engaging portion 353. Consequently, displacement of the puncture member 3 with respect to the insertion portion 71, more particularly, rotation of the puncture member 3 with respect to the insertion portion 71, displacement of the puncture member 3 in a widthwise direction with respect to the insertion portion 71 or the like, is inhibited, and puncture of the puncture member 3 into a living body can be performed with increased smoothness.

The puncture member 3 has been described above. A center angle θ4 (refer to FIG. 9) of the puncture member 3 is not restricted particularly and is appropriately set in accordance with various conditions. However, the center angle θ4 is set such that the needle body 35 can enter the body of a patient via one of the inguinal regions, pass between the urethra and the vagina and leave the body via the other inguinal region. In accordance with an exemplary embodiment, for example, the center angle θ4 preferably is approximately 150 degrees to 270 degrees, more preferably is approximately 170 degrees to 250 degrees, and most preferably is approximately 190 degrees to 230 degrees.

The constituent materials of the main body 31 and the needle body 35 preferably are such rigid materials as to maintain the shape and the internal space of them in a state where the puncture member 3 is inserted in a living body. As such rigid materials, for example, various resin materials such as polyethylene, polyimide, polyamide, polyester elastomer or polypropylene or various metal materials such as stainless steel, aluminum or aluminum alloys, titanium or titanium alloys can be used.

Note that, where the main body 31 and the needle body 35 are configured not only by adopting rigid materials but by adopting other materials than rigid materials, the wall may be reinforced with a reinforcement member. For example, a braid of high strength may be embedded in the wall to maintain the shape and the internal space in the state in which the puncture member 3 is inserted in a body. Another example of the reinforcement member is a spiral body, which is embedded in the wall of the main body 31, by which flexibility can be provided while the internal space is maintained to such an extent that an inserted article can be slidably moved therein.

In accordance with an exemplary embodiment, the main body 31 preferably is optically transparent so that the inside thereof can be visually observed from the outside, which makes it possible, for example, to simply confirm from the outside whether the distal portion 711 of the insertion portion 71 inserted in the inside of the main body 31 is in engagement with the engaging portion 353, whether the string 341 has not been cut, and so on.

In the puncture apparatus 1, such a puncture member 3 (main body 31) as described above and the insertion portion 71 which is inserted in the main body 31 cooperatively configure a medical tube assembly 10, and use of the puncture apparatus 1 is started in a state in which the members configure the medical tube assembly 10.

In accordance with an exemplary embodiment, the frame 2 holds the operation member 7, on which the puncture member 3 is mounted, for rotary movement and has the insertion tool 6 fixed thereto in a freely detachable manner. Such a frame 2 as just described has a function of determining a puncture route by the needle body 35 when the puncture member 3 punctures the living body tissue. In particular, the frame 2 determines the positional relationship of the puncture member 3, urethral-insertion tool 4 and vaginal-insertion tool 5 such that, when the puncture member 3 punctures the living body tissue, the needle body 35 passes between the urethral-insertion tool 4 and the vaginal-insertion tool 5 without colliding with any of the insertion tools.

As depicted in FIGS. 2 and 3, the frame 2 can include a bearing portion 21 for bearing the shaft portion 73 of the operation member 7, a guide portion 22 for guiding the puncture member 3, an interlock portion 23 for interlocking the bearing portion 21 and the guide portion 22 to each other, and a fixing portion 24 to which the insertion tool 6 is to be fixed.

In accordance with an exemplary embodiment, the bearing portion 21 is positioned on the proximal side of the puncture apparatus 1 and extends in a direction substantially orthogonal to the axis J1. The bearing portion 21 has a through-hole 211 formed on the axis J1 thereof, and the shaft portion 73 is inserted for rotary movement in the through-hole 211. As a result, the operation member 7 is supported on the frame 2 so as to move rotationally around the axis J1.

The guide portion 22 is positioned on the distal side of the puncture apparatus 1 and disposed in an opposing relationship to the bearing portion 21. As depicted in FIG. 9, the guide portion 22 is positioned on the inner side of the puncture member 3 and is configured such that it has an arcuate shape extending along the puncture member 3 and supports the puncture member 3 from the inner side. As depicted in FIG. 10, the puncture member 3 is disposed on the guide portion 22 such that the back face A4 is positioned on the distal side and the front face A3 is positioned on the proximal side.

In accordance with an exemplary embodiment, the interlock portion 23 interlocks the bearing portion 21 and the guide portion 22 to each other. The interlock portion 23 has a form of a bar extending substantially in parallel to the axis J1. The interlock portion 23 functions also as a grasping portion. An operator can use the puncture apparatus 1 in a stable state by grasping the interlock portion 23.

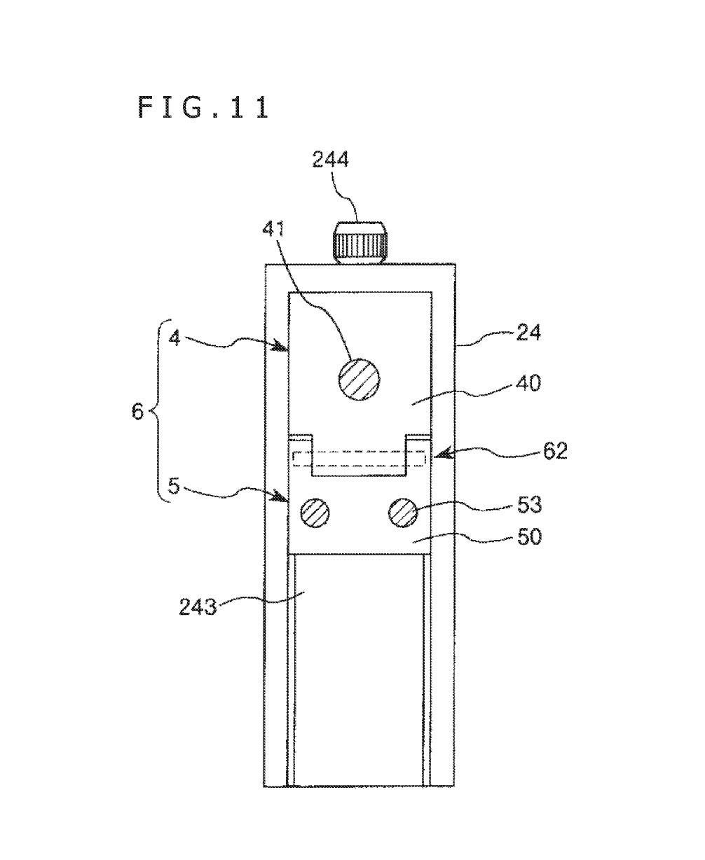

The fixing portion 24 is disposed in an opposing relationship to the interlock portion 23 with the axis J1 interposed therebetween. The fixing portion 24 has a recess 243 in which to fit supporting portions 40 and 50 hereinafter described of the insertion tool 6, and a male screw 244 as depicted in FIG. 11. With such a fixing portion 24 as just described, the insertion tool 6 can be fixed to the fixing portion 24 by fitting the supporting portions 40 and 50 into the recess 243 and then tightening the male screw 244 into the supporting portion 40.

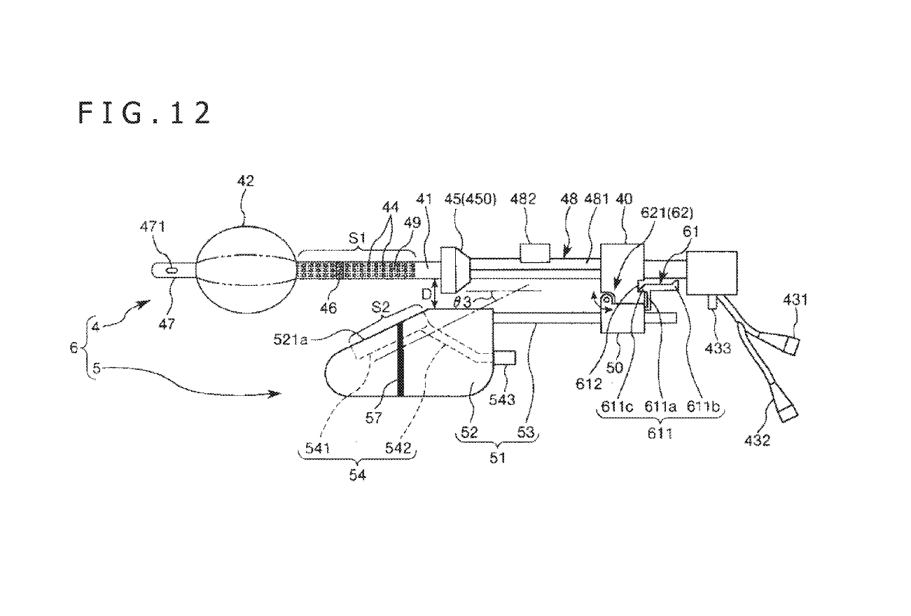

As depicted in FIG. 12, the insertion tool 6 has the urethral-insertion tool 4 and the vaginal-insertion tool 5.

The urethral-insertion tool 4 has an elongated urethral-insertion portion 41 for being inserted up to the middle thereof into the urethra, and the supporting portion (urethral-insertion portion supporting portion) 40 for supporting the urethral-insertion portion 41. As regards the constituent materials of the urethral-insertion portion 41 and the supporting portion 40, there is no particular restriction, and various metal materials such as, for example, stainless steel, aluminum or aluminum alloys, or titanium or titanium alloys or various resin materials can be used.

Further, the length of the urethral-insertion portion 41 (portion on the distal side from the supporting portion 40) is not restricted particularly but is suitably set depending upon the length of the urethra, the shape of the bladder and so forth of the patient. Since the length of the urethra of most women is approximately 30 mm to 50 mm, the length of the urethral-insertion portion 41 is preferably set to approximately 50 mm to 100 mm with reference to the length of the urethra.

In accordance with an exemplary embodiment, the urethral-insertion portion 41 can have a straight tube shape. At a distal portion of such a urethral-insertion portion 41 as just described, a balloon (attachment portion) 42 which is an expandable/contractible expansion body, and a urine drainage portion 47.

The balloon 42 is disposed such that it is positioned in the bladder when the urethral-insertion portion 41 is inserted in the urethra. The balloon 42 extends in the urethral-insertion portion 41 and is connected to a balloon port 431 provided at a proximal portion of the urethral-insertion portion 41. A balloon expansion tool such as a syringe can be connected to the balloon port 431, and if a working fluid (liquid such as physiological saline solution, or a gas or the like) is supplied into the balloon 42 from the balloon expansion tool, then the balloon 42 is expanded. In contrast, if the working fluid is extracted from the balloon 42 by the balloon expansion tool, then the balloon 42 is contracted. Note that in FIG. 12, the state in which the balloon 42 is contracted is indicated by an alternate long and short dashes line and the state in which the balloon 42 is expanded is indicated by a solid line.

The urine drainage portion 47 is used to drain the urine in the bladder in a state in which the urethral-insertion portion 41 is inserted in the urethra. A urine drain hole 471 is provided in the urine drainage portion 47 such that it extends between the inside and the outside of the urine drainage portion 47. Further, the urine drain hole 471 extends through the inside of the urethral-insertion portion 41 and is connected to a urine drain port 432 provided at a proximal portion of the urethral-insertion portion 41. Therefore, the urine introduced from the urine drain hole 471 can be drained from the urine drain port 432.

The balloon 42 and the urine drainage portion 47 can be configured, for example, from a double lumen.

In accordance with an exemplary embodiment, a plurality of suction holes 44 can be located in the urethral-insertion portion 41 on a proximal side of the balloon 42, and more particularly, for example, the plurality of suction holes 44 can be located at a mid-portion of the urethral-insertion portion 41. The plurality of suction holes 44 can be formed as a suction portion for absorbing a urethral wall to the urethral-insertion portion 41.

The plurality of suction holes 44 is disposed over an overall area in a circumferential direction of the urethral-insertion portion 41. The suction holes 44 are connected to a suction port 433 provided on the supporting portion 40 through the inside of the urethral-insertion portion 41. A suction device such as a pump can be connected to the suction port 433, and if the suction device is rendered operative in a state in which the urethral-insertion portion 41 is inserted in the urethra, then the urethral wall can be absorbed and fixed to the suction holes 44. In accordance with an exemplary embodiment, for example, by providing the plurality of suction holes 44 over an overall area in a circumferential direction of the urethral-insertion portion 41 as in the present embodiment, the urethral wall can be absorbed and fixed over a wide range thereof to the urethral-insertion portion 41.

Note that the number of the suction holes 44 is not restricted particularly, and for example, only one suction hole may be provided. Further, the layout of the suction holes 44 is not restricted particularly, and, for example, the suction holes 44 may be formed in only part of the urethral-insertion portion 41 in the circumferential direction.

If the urethral-insertion portion 41 is pushed in to the inside of the body (to the distal side of the urethral-insertion portion 41) in the state in which the urethral wall is absorbed and fixed to the urethral-insertion portion 41 in this manner, then the urethra and the bladder are pushed in to the inside of the body together with this. Consequently, the bladder can be displaced to a position at which it does not overlap with a puncture route of the puncture member 3. Therefore, a greater puncture route can be secured for the puncture member 3, and puncture of the puncture member 3 can be performed relatively accurately and safely.

Further, a visually observable marker (detection portion) 46 can be provided, for example, at an approximate mid-portion (or halfway) between the ends of the urethral-insertion portion 41 on the proximal side of the balloon 42. In accordance with an exemplary embodiment, the marker 46 can be used to detect the distance between the bladder and the urethral orifice, or in other words, the length of the urethra.

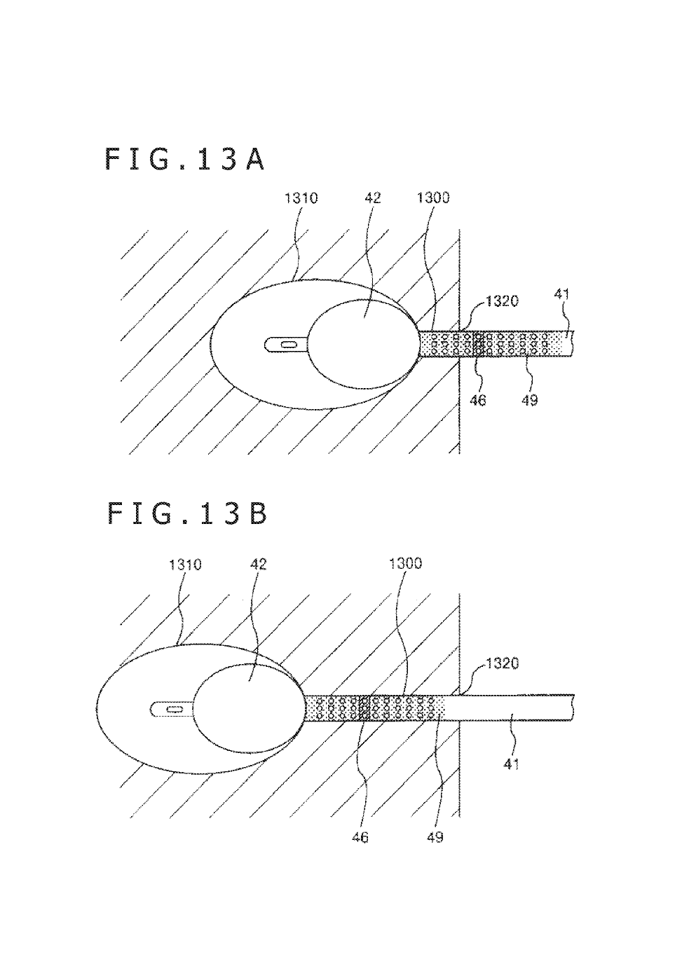

Describing more particularly, where the separated distance between a bladder 1310 and a urethral orifice 1320 is smaller than a predetermined distance as depicted in FIG. 13A, in a state in which the urethral-insertion portion 41 is inserted in a urethra 1300 and the balloon 42 is expanded until it is attached to the bladder neck, the marker 46 is exposed from the urethral orifice 1320. In contrast, if the separated distance between the bladder 1310 and the urethral orifice 1320 is greater than the predetermined distance as depicted in FIG. 13B, then in the state in which the urethral-insertion portion 41 is inserted in the urethra 1300 and the balloon 42 is expanded until it is attached to the bladder neck, the marker 46 is positioned in the urethra 1300 and is not exposed from the urethral orifice 1320.

If the marker 46 is exposed from the urethral orifice 1320 as depicted in FIG. 13A, then it can be estimated that the distance between the bladder 1310 and the urethral orifice 1320 is short and a sufficient space for allowing puncture by the puncture member 3 does not exist between them. Therefore, in this case, it can be decided that puncture by the puncture member 3 cannot be performed.

In contrast, if the marker 46 is not exposed from the urethral orifice 1320 as depicted in FIG. 13B, then it can be estimated that the distance between the bladder 1310 and the urethral orifice 1320 is sufficiently great and a sufficient space for allowing puncture by the puncture member 3 exists between them. Therefore, in this case, it can be decided that puncture by the puncture member 3 can be performed.

In this manner, by providing the marker 46, it is possible to decide whether or not puncture by the puncture member 3 through the puncture apparatus 1 is possible, or in other words, whether or not the puncture apparatus 1 can be applied to the patient. Especially, by disposing the balloon 42 and causing the balloon 42 to attach to the bladder neck, the separated distance between the bladder 1310 and the urethral orifice 1320 can be detected accurately, and the aforementioned decision can be performed with enhanced accuracy.

The configuration of the marker 46 is not restricted particularly only if the operator can visually observe the marker 46 from the outside. For example, the marker 46 may be configured from a portion colored in a different color from those of surrounding elements (distal side and proximal side with respect to the marker 46) or may be configured from a recess or a projection.

Alternatively, the marker 46 may be graduations to which distances from the proximal end of the balloon 42 are inscribed. In this case, since whether or not puncture by the puncture member 3 is possible cannot be decided depending upon whether or not the marker 46 is exposed from the urethral orifice 1320 as in the present embodiment, the length of the urethra 1300 may be measured by the graduations to decide whether or not puncture by the puncture member 3 is possible depending upon the length.

Further, as depicted in FIGS. 12, 13A and 13B, a sliding resistance amplification portion 49 for contacting with the urethral wall (tissue in the urethra) to raise the sliding resistance with respect to the urethra is provided at a distal portion of the urethral-insertion portion 41. Although the bladder 1310 is displaced sufficiently to a position at which it does not overlap with the puncture route of the puncture member 3 by absorbing and fixing the urethral wall to the urethral-insertion portion 41 during a procedure in which the puncture apparatus 1 is used and pushing in the urethral-insertion portion 41 to the inside of the body as described above, if the absorption of the urethral wall by the suction holes 44 thereupon is not sufficient, then the urethral wall may be come off from the suction holes 44, resulting in failure to push in the bladder 1310 sufficiently. Therefore, by providing the sliding resistance amplification portion 49 on the urethral-insertion portion 41 so that the urethral-insertion portion 41 is less likely to slip with respect to the urethral wall as in the present embodiment, even in a case in which absorption by the suction holes 44 is not sufficient, the bladder 1310 can be pushed in to the inside of the body, or even in another case in which absorption by the suction holes 44 is sufficient, the bladder 1310 can be pushed in to the inside of the body with a higher degree of certainty by a synergetic effect. Therefore, puncture by the puncture member 3 can be performed relatively safely.

The configuration of the sliding resistance amplification portion 49 is not restricted particularly if the aforementioned effect can be achieved. In the present embodiment, the sliding resistance amplification portion 49 is configured from fine recesses/projections (rough surface) formed on the surface of the urethral-insertion portion 41. Consequently, the configuration of the sliding resistance amplification portion 49 can be simplified. Note that the aforementioned recesses/projections signify that only it is necessary for the configuration of the sliding resistance amplification portion 49 to have at least one of recesses and projections.

Further, the sliding resistance amplification portion 49 can be provided over an overall area in a circumferential direction of the urethral-insertion portion 41. Consequently, since the area of the sliding resistance amplification portion 49 can be made greater, the sliding resistance with respect to the urethra 1300 can be increased more. Note that the sliding resistance amplification portion 49 can be formed by embossing, sandblasting, laser processing or the like.

Further, the sliding resistance amplification portion 49 is provided in an overlapping relationship with a region S1 in which the suction holes 44 are formed. In other words, the suction holes 44 are formed at the sliding resistance amplification portion 49. If the urethral wall is absorbed by the suction holes 44, then the urethral wall bites into and is caught by the suction holes 44. Since such an anchor effect as just described is demonstrated in the sliding resistance amplification portion 49, the sliding resistance of the urethral-insertion portion 41 with respect to the urethra can be increased more. Further, since the sliding resistance amplification portion 49 is provided in an overlapping relationship with the region S1, the sliding resistance amplification portion 49 and the region S1 can be disposed widely, and therefore, higher effects can be demonstrated by both of them.

Further, a tubular marker (positioning portion) 45 is provided for sliding movement on the urethral-insertion portion 41. Further, a restriction portion 48 can be mounted on the urethral-insertion portion 41 between the marker 45 and the supporting portion 40 such that, when the marker 45 is attached to the restriction portion 48, further sliding movement of the marker 45 toward the proximal side is restricted. The restriction portion 48 can be removed from the urethral-insertion portion 41. If the restriction portion 48 is removed from the urethral-insertion portion 41, then the marker 45 can be slidably moved further to the proximal side.

The configuration of the restriction portion 48 is not restricted particularly only if the aforementioned effect can be achieved. In the present embodiment, the restriction portion 48 has a main body 481 having a substantially C-like semi-cylindrical shape as a transverse sectional shape thereof, and a knob 482 provided on the main body 481. The main body 481 is mounted on the urethral-insertion portion 41, for example, in a state in which it has a diameter increased a little so that it may not be removed from the urethral-insertion portion 41 by its own weight or may not slidably move with respect to the urethral-insertion portion 41.

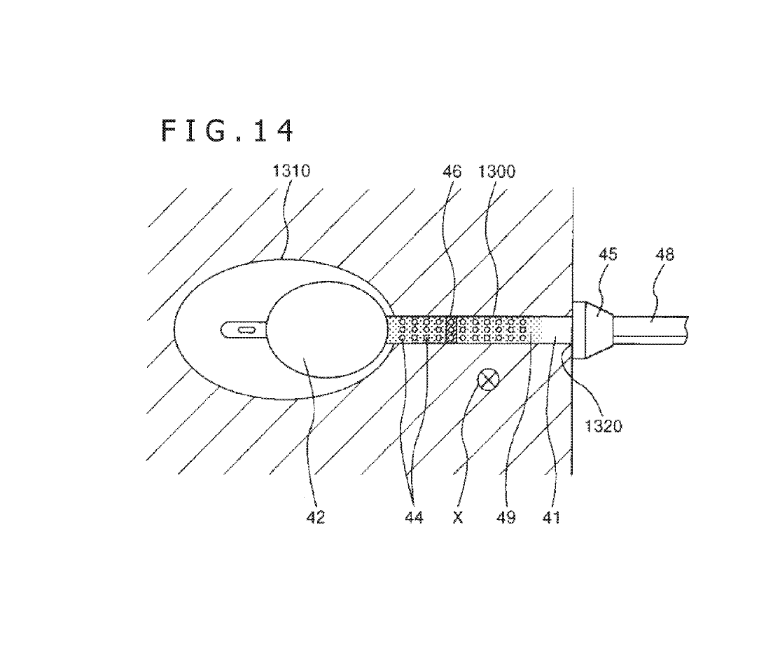

The marker 45 is positioned such that, in a state in which it is attached to the restriction portion 48 and is restricted from slidably moving further to the proximal side (state illustrated in FIG. 12), it has a predetermined relative positional relationship with the puncture route of the puncture member 3. Therefore, if the urethral-insertion portion 41 is inserted into the urethra 1300 and the marker 45 can be positioned at the urethral orifice 1320 (or in the proximity of the urethral orifice 1320) as depicted in FIG. 14, then a puncture route X of the puncture member 3 can be positioned with respect to the urethral orifice 1320 (or in the proximity of the urethral orifice 1320). By positioning the puncture route X with respect to the urethral orifice 1320 (or in the proximity of the urethral orifice 1320) in this manner, the accuracy in positioning can be enhanced and accurate puncture by the puncture member 3 can be performed in comparison with an alternative case in which the puncture route X is positioned, for example, with respect to the bladder 1310. Here, the “predetermined relative positional relationship” can be regarded as such a positional relationship that, for example, in a state in which the marker 45 is positioned at the urethral orifice 1320, the puncture route X of the puncture member 3 does not overlap with the bladder 1310.

Further, since the marker 45 has an outer diameter greater than that of the urethral-insertion portion 41 and projects to the outer side of the urethral-insertion portion 41, it cannot be inserted into the urethra 1300. Therefore, if the urethral-insertion portion 41 is inserted into the urethra 1300, then the marker 45 is attached, halfway of the insertion, to the urethral orifice 1320 (or the tissue around the urethral orifice 1320, this similarly applies also to the following description) and the marker 45 can be positioned at the urethral orifice 1320 naturally. By utilizing a physical catch in this manner, the marker 45 can be positioned at the urethral orifice 1320 with relative certainty. Further, when the marker 45 is attached to the urethral orifice 1320, further insertion of the urethral-insertion portion 41 into the urethra 1300 is restricted, and therefore, excessive insertion of the urethral-insertion portion 41 into the urethra 1300 can be prevented and the safety of the puncture apparatus 1 can be enhanced.

Such a marker 45 as described above has, in addition to such a function of positioning the puncture route of the puncture member 3 with respect to the urethral orifice 1320 as described above, another function as an inflow reduction portion 450 for reducing inflow of air into the urethra 1300 through the urethral orifice 1320 in a state in which the urethral-insertion portion 41 is inserted in the urethra 1300. Describing more particularly, in a state in which the urethral-insertion portion 41 is inserted in the urethra 1300 and the marker 45 is attached to the urethral orifice 1320 as depicted in FIG. 14, the urethral orifice 1320 is covered with and closed up by the marker 45. Therefore, it can be difficult for air to flow into the urethra 1300 through the urethral orifice 1320, and inflow of air into the urethra 1300 (suction holes 44) through the urethral orifice 1320 can be reduced. As a result, absorption of the urethral wall by the suction holes 44 can be performed with a higher degree of certainty and effectiveness.

Since the marker 45 serves also as the inflow reduction portion 450 in this manner, the configuration of the urethral-insertion tool 4 can be simplified and also an operation of the urethral-insertion tool 4 can be simplified. Further, the number of component parts can be reduced, which can contribute to a reduction in cost.

Now, inclination between the urethral-insertion portion 41 and the puncture member 3 is described. As depicted in FIG. 3, although the inclination angle (in other words, the angle defined by the axes J1 and J2) θ2 of the plane f9 (plane f1) with respect to the plane f2 orthogonal to the axis J2 of the urethral-insertion portion 41 is not restricted particularly, for example, it preferably is approximately 20 degrees to 60 degrees, more preferably is approximately 30 degrees to 45 degrees, and most preferably is approximately 35 degrees to 40 degrees. By this, puncture by the puncture member 3 can be performed relatively easily. Further, the puncture distance by the puncture member 3 can be made shorter.

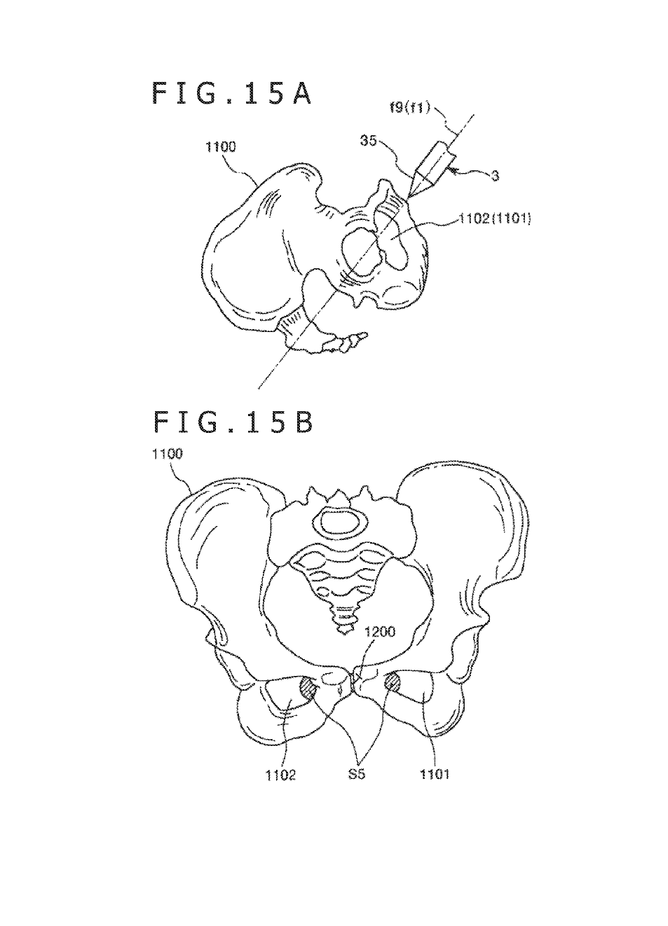

Describing more particularly, by setting the inclination angle θ2 within the aforesaid range, the puncture member 3 can capture left and right obturator foramens 1101 and 1102 of a pelvis 1100 widely on a plane as depicted in FIG. 15A, and a wide puncture space can be secure for the puncture member 3. In other words, in a state in which the patient assumes a predetermined posture (lithotomy position), the puncture member 3 can puncture in a direction comparatively near to the vertical direction relative to the obturator foramens 1101 and 1102. Therefore, the puncture by the puncture member 3 can be performed relatively easily.

In addition, by causing the puncture member 3 to puncture in a direction comparatively near to the vertical direction relative to the obturator foramens 1101 and 1102, the puncture member 3 passes a shallow portion of the tissue. Therefore, the needle body 35 of the puncture member 3 can pass between the left and right obturator foramens 1101 and 1102 taking a shorter course. Therefore, as depicted in FIG. 15B, the puncture member 3 can pass those zones in the obturator foramens 1101 and 1102 rather near to a pubic symphysis 1200, preferably, safety zones S5. Since the safety zones S5 are regions which include a comparatively small number of nerves and blood vessels to which damage should be avoided, the puncture member 3 passes the safety zones S5 so that the puncture can be performed by the puncture member 3 safely. Therefore, a less invasive procedure can be realized, and the burden on the patient can be reduced.

In this manner, by setting the inclination angle θ2 within the aforesaid range, the puncture of the patient by the puncture member 3 can be performed appropriately. Further, the puncture at the aforementioned angle makes it easier to aim at the tissue between a middle urethra (which refers to a middle portion of the urethra in the longitudinal direction) and the vagina. Here, since the tissue between the middle urethra and the vagina is a position suitable as a region into which the implant 9 is to be embedded to carry out treatment for urinary incontinence, effective treatment can be performed by targeting the tissue between the middle urethra and the vagina.

In contrast, where the inclination angle θ2 is smaller than the aforesaid lower limit or greater than the aforesaid upper limit, depending upon individual differences of the patient, the posture of the patient during the procedure or the like, it sometimes occurs that the puncture member 3 cannot capture the obturator foramens 1101 and 1102 widely on a plane or the puncture route cannot be made sufficiently short.

The configuration of the urethral-insertion tool 4 has been described above. In such a urethral-insertion tool 4 as described above, the urethral-insertion portion 41 may be configured so as not to be able to slidably move with respect to the supporting portion 40 or so as to be able to slidably move with respect to the supporting portion 40. Where the urethral-insertion portion 41 is configured for sliding movement, the urethral-insertion portion 41 may be configured such that, for example, if a screw (not depicted) provided on the supporting portion 40 is loosened, then the urethral-insertion portion 41 is permitted to slidably move with respect to the supporting portion 40, but if the screw is tightened, then the urethral-insertion portion 41 is placed into a state in which it is fixed to the supporting portion 40. With the configuration just described, since the length of the urethral-insertion portion 41 can be adjusted, the urethral-insertion tool 4 is improved further in convenience in use. Note that this similarly applies also to the vaginal-insertion tool 5 hereinafter described.

Further, while, in the puncture apparatus 1, the urethral-insertion tool 4 is fixed to the frame 2 such that the inclination angle θ2 is fixed, the puncture apparatus 1 is not limited to this, but the inclination angle θ2 may be variable. By this, the inclination angle θ2 can be adjusted in accordance with the patient, and therefore, the puncture apparatus 1 is further improved in convenience in use.

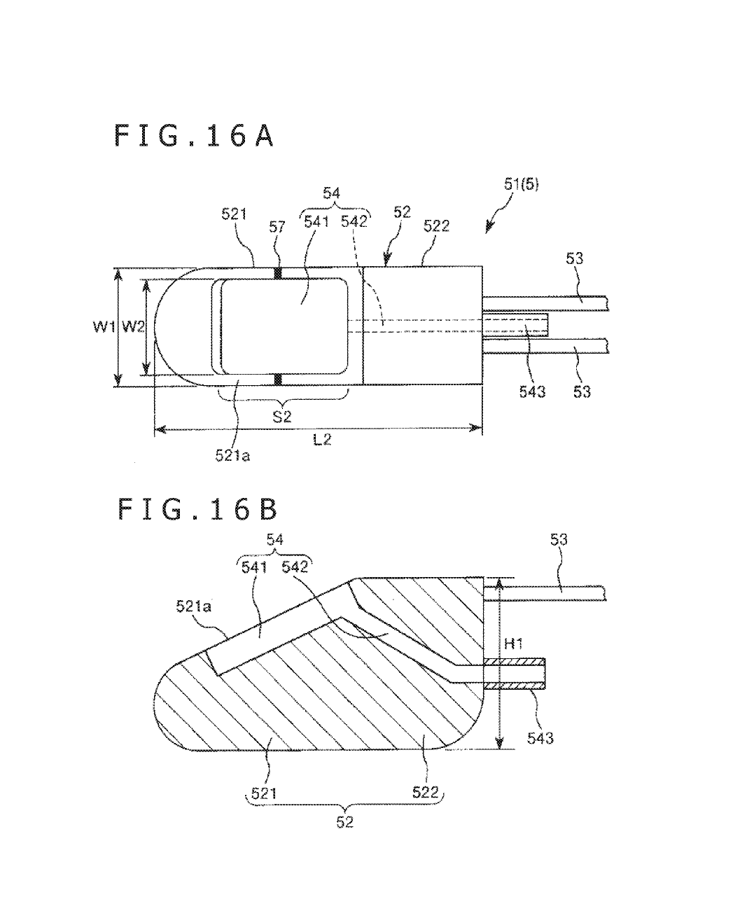

As depicted in FIGS. 12, 16A and 16B, the vaginal-insertion tool 5 has an elongated vaginal-insertion portion 51 for being inserted up to the middle thereof into the vagina, and a supporting portion (vaginal-insertion portion supporting portion) 50 for supporting the vaginal-insertion portion 51. Further, the vaginal-insertion portion 51 has a distal portion 52 positioned on the distal side and a shaft portion 53 connected to a proximal portion of the distal portion 52, the shaft portion 53 being supported by the supporting portion 50.

As the constituent materials of the vaginal-insertion portion 51 and the supporting portion 50, there is no particular restriction, and various metal materials such as, for example, stainless steel, aluminum or aluminum alloys, or titanium or titanium alloys or various resin materials can be used, like in the case of the urethral-insertion tool 4 (the urethral-insertion portion 41 and the supporting portion 40).

The distal portion 52 is a portion for being inserted into the vagina. Although the length L2 of the distal portion 52 is not restricted particularly, for example, it preferably is approximately 20 mm to 100 mm, and more preferably is approximately 30 mm to 60 mm. Further, although the width W1 of the distal portion 52 is not restricted particularly, it preferably is approximately 10 mm to 50 mm, and more preferably is approximately 20 mm to 40 mm. By adopting such a length (L2) and a width (W1) as given above, the distal portion 52 is provided with a shape and a size suitable for most vaginas. Therefore, the stability of the puncture apparatus 1 in the mounted state can be enhanced and the burden on the patient can be reduced.

Further, the distal portion 52 has a substantially fixed width as a whole and has a rounded distal portion. Further, the distal portion 52 has a tapering portion 521 positioned on the distal side and having a height gradually increasing toward the proximal side, and an inflow reduction portion 522 positioned on the proximal side of the tapering portion 521 and having a substantially fixed height. Note that the inflow reduction portion 522 has a function of reducing inflow of air into the vagina through the vaginal orifice in a state in which the distal portion 52 is inserted in the vagina.

In the tapering portion 521, an upper face (face on the urethral-insertion portion 41 side) 521a is inclined with respect to the urethral-insertion portion 41 such that it is spaced away from the urethral-insertion portion 41 toward the distal end. Consequently, in comparison with an alternative case in which the upper face 521a is not inclined, the positional relationship between the urethral-insertion portion 41 and the distal portion 52 can be made closer to the actual positional relationship between the urethra and the vagina. Therefore, in the mounted state, the puncture apparatus 1 can be held stably on the patient and the burden on the patient can be reduced.

Although an inclination angle θ3 of the upper face 521a with respect to the urethral-insertion portion 41 is not restricted particularly, it preferably is, for example, approximately 0 degrees to 45 degrees, and more preferably is approximately 0 degrees to 30 degrees. This setting can help enable the aforesaid effects to be exhibited more remarkably. In contrast, if the inclination angle θ3 is smaller than the aforesaid lower limit value or is greater than the aforesaid upper limit value, then depending upon the individual differences of the patient, the posture during the procedure or the like, the vagina or the urethra is deformed unnaturally in the mounted state, and the puncture apparatus 1 may not be held stably.

Meanwhile, in the distal portion 52, a suction hole 54 is provided as a suction portion for absorbing the vaginal wall to the distal portion 52.

The suction hole 54 has a bottomed recess 541 which is open to the upper face 521a of the tapering portion 521. Consequently, as hereinafter described, the vaginal anterior wall can be absorbed to the distal portion 52. Further, the suction hole 54 has a connection hole 542 provided in the distal portion 52 for connecting the bottom face of the recess 541 and a suction port 543 provided at a proximal portion of the distal portion 52 to each other.

The suction port 543 is provided so as to be positioned outside a living body in the mounted state. To the suction port 543, a suction device such as a pump can be connected. If the suction device is rendered operative in a state in which the distal portion 52 is inserted in the vagina, then the vaginal anterior wall which is an upper face of the vaginal wall is absorbed and fixed to the recess 541. If the vaginal-insertion portion 51 is pushed in to the inner side of the body (distal side of the vaginal-insertion portion 51) in a state in which the vaginal anterior wall is absorbed and fixed to the recess 541, then the vaginal wall can be pushed in together with this. Therefore, the disposition or the shape of the vaginal wall can be arranged, and the puncture route for the puncture member 3 can be secured sufficiently. As a result, puncture by the puncture member 3 can be performed relatively accurately and safely.

Note that the number of suction holes 54 is not limited to one but a plurality of suction holes 54 may be provided. Where a plurality of suction holes 54 are provided, the disposition of the recess 541 is not limited particularly but the recesses 541 may be disposed in a juxtaposed relationship in the lengthwise direction or may be disposed in a juxtaposed relationship in a widthwise direction. Alternatively, the recesses 541 may be disposed in a juxtaposed relationship in both of the lengthwise direction and the widthwise direction and hence in a matrix or may be disposed irregularly.

As depicted in FIG. 12, a region S2 in which the recess 541 is formed is opposed to the region S1 in which the suction holes 44 are formed. Further, the puncture apparatus 1 is configured such that the needle tip of the puncture member 3 passes between the regions S1 and S2. As described hereinabove, since, in the region S1, the urethral wall is absorbed to the urethral-insertion portion 41 and, in the region S2, the vaginal wall is absorbed to the distal portion 52, between the regions S1 and S2, the urethral wall and the vaginal wall are spaced away from each other by a greater distance and with a relative higher degree of certainty. By passing the puncture member 3 through such a region as just described, the puncture member 3 can be operated to puncture with a higher degree of safety.

Note that the region S2 preferably extends substantially over an overall area in a widthwise direction of the upper face 521a as depicted in FIG. 16A. Although the width W2 of the region S2 is not restricted particularly, for example, it preferably is approximately 9 mm to 49 mm, and more preferably is approximately 19 mm to 39 mm. By this, the vaginal wall can be absorbed to the distal portion 52 with a higher degree of certainty without being influenced much by the shape of the vaginal wall.

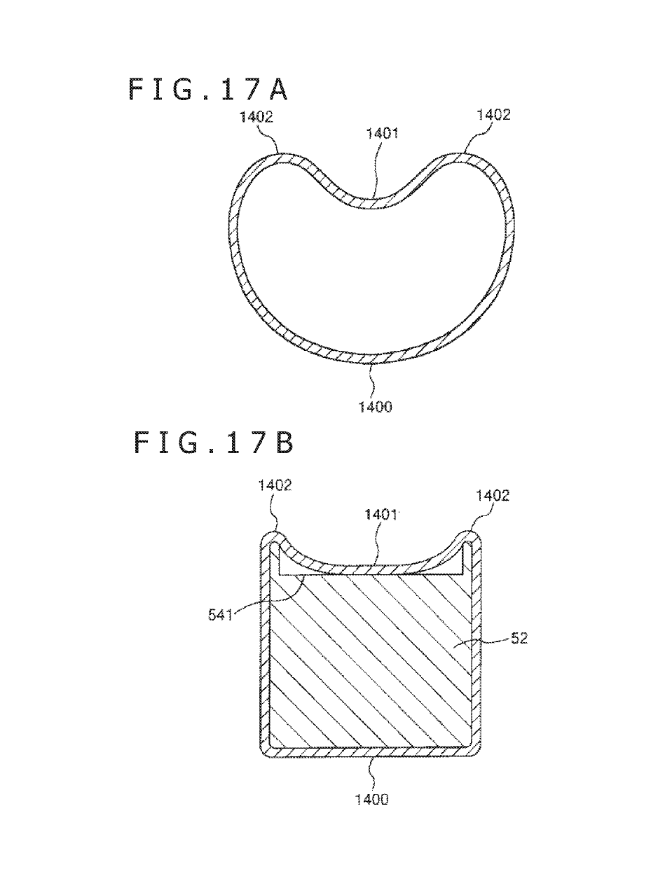

In accordance with an exemplary embodiment, for example, depending upon the patient, a central portion 1401 of the vaginal anterior wall extends downwardly into the vagina such that a vagina 1400 has depressed portions (regions also called “buttonholes”) 1402 on the opposite sides of the central portion 1401. Also in such a case as just described, not only the central portion 1401 but also the depressed portions 1402 can be absorbed with a higher degree of certainty as depicted in FIG. 17B. By absorbing the depressed portions 1402 to the distal portion 52 with a higher degree of certainty, the depressed portions 1402 can be spaced away from the urethra 1300, and the depressed portions 1402 can be effectively prevented from being punctured by the puncture member 3.

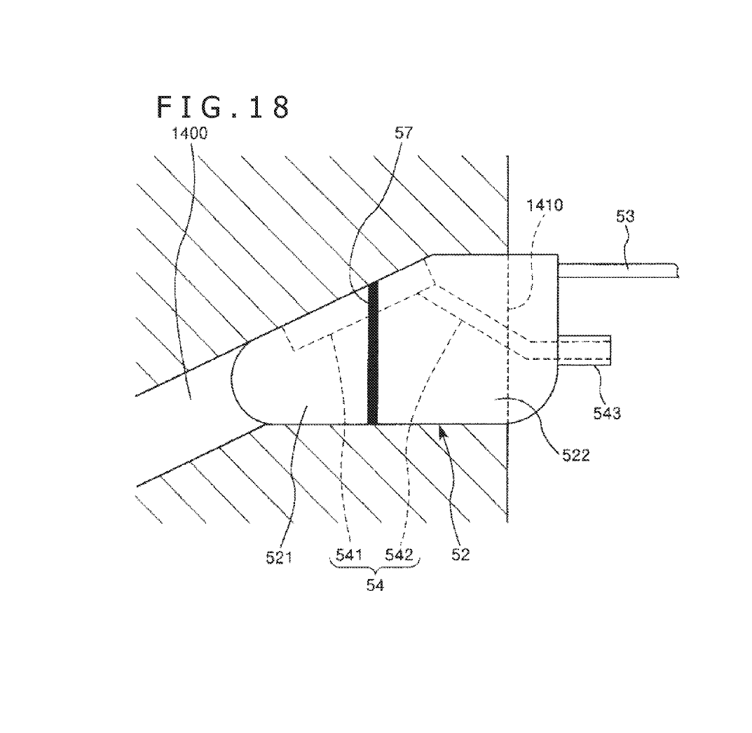

Here, in the state in which the distal portion 52 is inserted in the vagina 1400 as depicted in FIG. 18, the inflow reduction portion 522 is positioned in the proximity of a vaginal orifice 1410 of the vagina 1400 (on the proximal side with respect to the region of the vagina 1400 in which the recess 541 is positioned) and at the vaginal orifice 1410. The inflow reduction portion 522 is higher than the tapering portion 521 on the distal side therefrom and is longer at an overall circumference thereof (transverse area) than the tapering portion 521. Therefore, a portion of the vagina 1400 in the proximity of the vaginal orifice 1410 and the vaginal orifice 1410 extend by a great amount and are closely contacted with the outer periphery of the inflow reduction portion 522 strongly by the contracting force thereof. Consequently, inflow of air into the vagina 1400 (recess 541) through the vaginal orifice 1410 is reduced, and absorption of the vaginal wall by the suction hole 54 can be performed with a relatively higher degree of certainty and effectiveness.

Note that the shape of the inflow reduction portion 522 is not restricted to the shape in the present embodiment only if it can demonstrate the aforementioned effect, and for example, the inflow reduction portion 522 may be configured such that the height H1 thereof gradually increases toward the proximal side. In this case, the upper face of the inflow reduction portion 522 may be a face continuing to and having an inclination same as that of the upper face 521a of the tapering portion 521 or may be a discontinuous face having a different inclination.

Further, at the distal portion 52, a marker 57 such that the puncture route of the puncture apparatus 1 can be confirmed thereby is provided. Since the marker 57 is provided such that the puncture member 3 punctures a region between the vaginal wall positioned thereabove and the urethral wall, by confirming the position of the marker 57, the puncture route of the puncture member 3 can be confirmed relatively easily. Consequently, the operability and the safety of the puncture apparatus 1 are improved. The marker 57 is preferably provided on at least one of the bottom face and a side face of the distal portion 52. Since the bottom face or the side face is a face, which can be visually confirmed readily by the operator even in a state in which the distal portion 52 is inserted in the vagina, by providing the marker 57 at such a position as just described, the puncture route of the puncture member 3 can be confirmed. Further, the insertion depth of the distal portion 52 in the vagina can be confirmed. Only it is necessary for such a marker 57 as just described to be visually observed from the outside, and it can be configured, for example, from a colored portion, a recess or projection or the like.

Although the separated distance D between such a distal portion 52 and a urethral-insertion portion 41 as described above is not restricted particularly, for example, it preferably is approximately 5 mm to 40 mm with reference to the separated distance between the urethral orifice and the vaginal orifice of most women.

In accordance with an exemplary embodiment, the shaft portion 53 has a form of a thin bar extending substantially in parallel to the urethral-insertion portion 41. Although the length of the shaft portion 53 (separated distance between the distal portion 52 and the supporting portion 50) is not restricted particularly, for example, it preferably is substantially equal to or smaller than approximately 100 mm and more preferably is approximately 20 mm to 50 mm. By this, the shaft portion 53 can be made with an appropriate length, and the operability of the puncture apparatus 1 is improved. If the length of the shaft portion 53 exceeds the aforesaid upper limit value, then depending upon the configuration and so forth of the frame 2, the center of gravity of the puncture apparatus 1 is displaced by a great distance from the patient, and the stability of the puncture apparatus 1 in the mounted state may be degraded.

The urethral-insertion tool 4 and the vaginal-insertion tool 5 have been described. The insertion tool 6 having such a urethral-insertion tool 4 and a vaginal-insertion tool 5 as described above further can have a maintaining portion 61 for maintaining a relative positional relationship between the urethral-insertion portion 41 and the vaginal-insertion portion 51, and a displacement portion 62, which can move the distal portion 52 of the vaginal-insertion portion 51 closer to the urethral-insertion portion 41 than the relative positional relationship maintained by the maintaining portion 61.

As depicted in FIG. 12, the displacement portion 62 has a hinge portion 621, and the supporting portions 40 and 50 are interlocked with each other for rotary movement by the hinge portion 621. By interlocking the supporting portion 40 with the supporting portion 50 for rotary movement in this manner, the displacement portion 62 of a simple configuration can be obtained, and further, an operation for moving the distal portion 52 toward the urethral-insertion portion 41 can be performed.

Meanwhile, the maintaining portion 61 can assume a state in which it fixes the urethral-insertion tool 4 and the vaginal-insertion tool 5 to maintain the relative positional relationship therebetween and another state in which it cancels the fixation of the urethral-insertion tool 4 and the vaginal-insertion tool 5 and allows the vaginal-insertion tool 5 to be moved rotationally with respect to the urethral-insertion tool 4.

As depicted in FIG. 12, the maintaining portion 61 has an operation portion 611 provided on the supporting portion 50, and an engaging portion 612 provided on the supporting portion 40 for engaging with the operation portion 611. Further, the operation portion 611 has a shaft portion 611a which can be flexion-deformed (elastically deformed), a knob 611b interlocked with the shaft portion 611a, and a pawl portion 611c provided at a distal portion of the knob 611b. The engaging portion 612 can be configured from a recess 612a formed on the supporting portion 40.

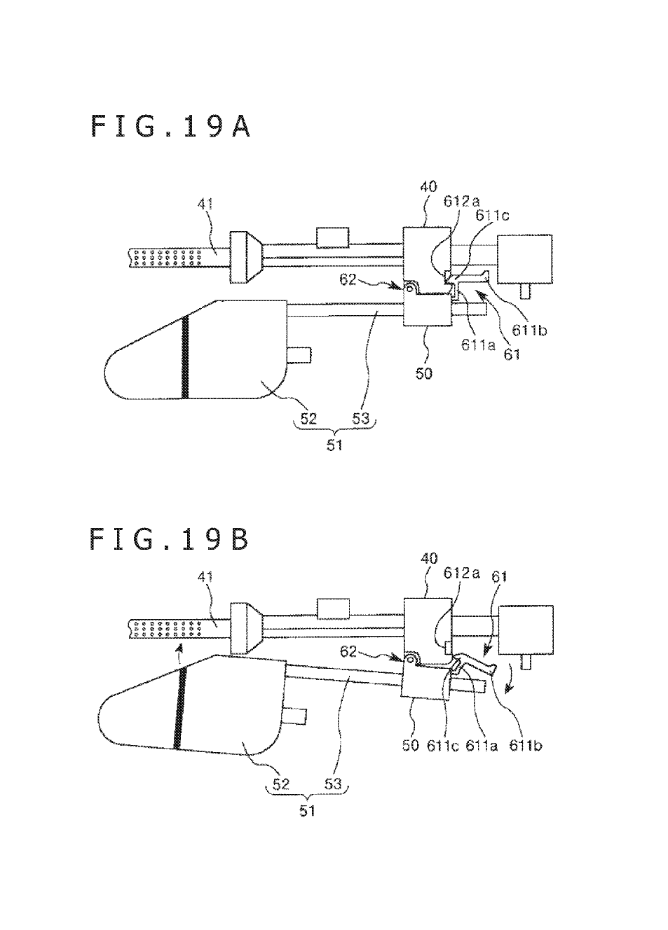

As depicted in FIG. 19A, by placing the pawl portion 611c into a state in which it is engaged with the recess 612a, the supporting portion 50 is fixed to the supporting portion 40, by which the relative positional relationship between the urethral-insertion tool 4 and the vaginal-insertion tool 5 can be maintained. On the other hand, by pulling the knob 611b downwardly to remove the pawl portion 611c from the recess 612a as depicted in FIG. 19B, the maintenance state is canceled and the vaginal-insertion tool 5 is placed into a state in which it can be moved rotationally with respect to the urethral-insertion tool 4. In the state in which the vaginal-insertion tool 5 can be moved rotationally, the vaginal-insertion portion 51 (distal portion 52) can be moved more closely to the urethral-insertion portion 41 than in the maintenance state depicted in FIG. 19A.

Note that, while the engaging portion 612 in the present embodiment is provided on the supporting portion 40 and the operation portion 611 is provided on the supporting portion 50, conversely the engaging portion 612 may be provided on the supporting portion 50 while the operation portion 611 is provided on the supporting portion 40. Further, the configuration of the operation portion 611 is not limited to that of the present embodiment if a similar effect can be demonstrated.

Further, the insertion tool 6 may further include a biasing portion (for example, a spring) for biasing the vaginal-insertion portion 51 to the urethral-insertion portion 41 side. By this, if the maintaining portion 61 is unlocked, then the vaginal-insertion portion 51 is moved automatically toward the urethral-insertion portion 41 side by the biasing force of the biasing portion. Therefore, an operation of the insertion tool 6 is further simplified.

Now, usage of the puncture apparatus 1, namely, a method (procedure) of embedding the implant 9 into a living body using the puncture apparatus 1, is described.

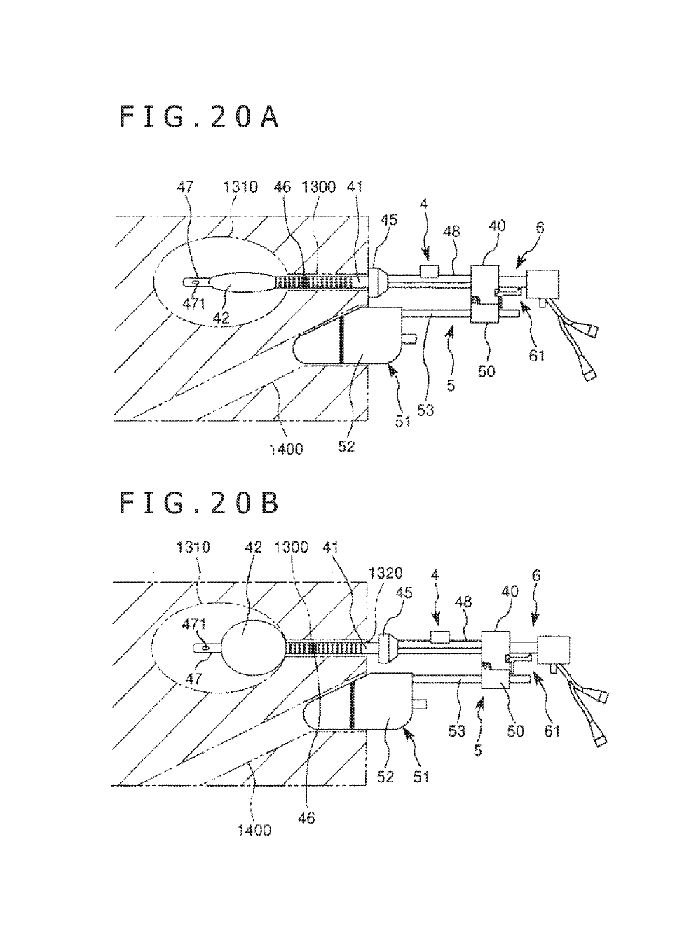

First, the patient is set to a lithotomy position on an operating table, and the insertion tool 6 is mounted on the patient as depicted in FIG. 20A.

In accordance with an exemplary embodiment, the urethral-insertion portion 41 of the urethral-insertion tool 4 is first inserted into the urethra 1300 of the patient and the vaginal-insertion portion 51 of the vaginal-insertion tool 5 is inserted into the vagina 1400 of the patient. By inserting the urethral-insertion portion 41 into the urethra 1300, the urethra 1300 is corrected to its predetermined shape (in the present embodiment, into a linear shape) by the urethral-insertion portion 41, and by inserting the vaginal-insertion portion 51 into the vagina 1400, the vagina 1400 is corrected to its predetermined shape by the vaginal-insertion portion 51 (distal portion 52).

Then, the balloon 42 disposed in the bladder 1310 is expanded and the urine is drained from within the bladder 1310 through the urine drain hole 471 as occasion demands. Then, as depicted in FIG. 20B, the urethral-insertion portion 41 is pulled to the outer side of the body (to the proximal side) until the balloon 42 attaches the bladder neck, and in this state, it is confirmed whether or not the marker 46 is exposed from the urethral orifice 1320. As described hereinabove, where the marker 46 is exposed from the urethral orifice 1320, it can be decided that the puncture apparatus 1 cannot be used, and at this point of time, the procedure is discontinued. In contrast, where the marker 46 is not exposed from the urethral orifice 1320, it can be decided that the puncture apparatus 1 can be used, and the procedure is continued.

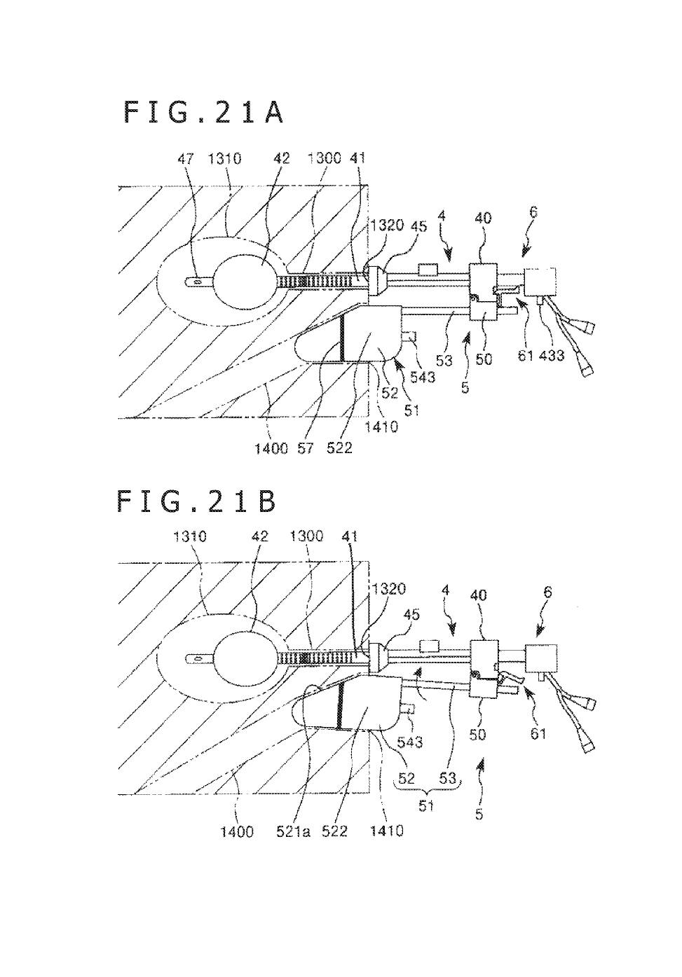

Then, the urethral-insertion portion 41 is pushed in to the inner side of the body until the marker 45 attaches the urethral orifice 1320 as depicted in FIG. 21A. Consequently, the puncture member 3 is positioned with respect to the urethral orifice 1320, and the puncture route of the puncture member 3 is defined. Here, by further confirming the puncture route of the puncture member 3 using the marker 57 provided at the distal portion 52 of the vaginal-insertion portion 51, a relatively safer procedure can be performed.

Then, suction devices are connected to the suction ports 433 and 543 and are rendered operative to suck the urethral wall to the suction holes 44 of the urethral-insertion portion 41 and absorb the vaginal wall to the suction hole 54 of the vaginal-insertion portion 51. As described hereinabove, since, at this time, on the urethra 1300 side, the marker 45 covers and closes up the urethral orifice 1320, inflow of air into the urethra 1300 through the urethral orifice 1320 is reduced, and the absorption of the urethral wall to the urethral-insertion portion 41 can be performed with a relative higher degree of certainty. Meanwhile, on the vagina 1400 side, since the vaginal orifice 1410 closely contacts with the inflow reduction portion 522, inflow of air into the vagina 1400 through the vaginal orifice 1410 can be reduced, and the absorption of the vaginal wall to the vaginal-insertion portion 51 can be performed with a relatively higher degree of certainty.

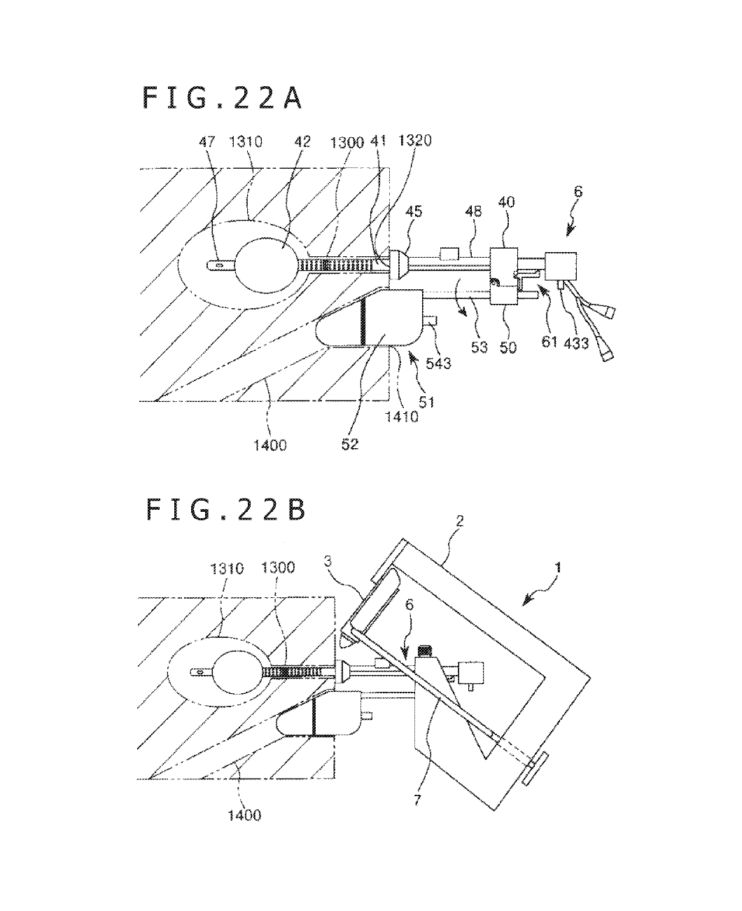

In accordance with an exemplary embodiment, for example, in the vaginal-insertion tool 5, the maintaining portion 61 is unlocked first as depicted in FIG. 21B to place the vaginal-insertion portion 51 into a state in which it can move rotationally with respect to the urethral-insertion portion 41. Then, the vaginal-insertion portion 51 is moved to the urethral-insertion portion 41 side. Consequently, the upper face 521a of the distal portion 52 and the vaginal anterior wall can be closely contacted with a higher degree of certainty with each other. In this state, by rendering the suction device connected to the suction port 543 operative, the vaginal wall can be absorbed to the vaginal-insertion portion 51 with a higher degree of certainty. After the vaginal wall is absorbed, the vaginal-insertion portion 51 is moved in a direction away from the urethral-insertion portion 41 as depicted in FIG. 22A, and the maintaining portion 61 is locked to fix the relative positional relationship of the vaginal-insertion portion 51 and the urethral-insertion portion 41. Consequently, the state in which the urethra 1300 and the vagina 1400 are sufficiently spaced away from each other can be maintained.

Upon such absorption as described above, for example, if the urethral wall is absorbed precisely to the urethral-insertion portion 41, then since the suction holes 44 is closed up with the urethral wall, the suction from the suction port 433 is stopped or weakened. Similarly, if the vaginal wall is precisely absorbed to the vaginal-insertion portion 51, then since the suction hole 54 is closed up with the vaginal wall, the suction from the suction port 543 is stopped or weakened. Therefore, the operator can confirm whether or not the urethral wall and the vaginal wall are precisely absorbed to the urethral-insertion portion 41 and the vaginal-insertion portion 51 from suction conditions (for example, magnitudes of sound generated by the suction) from the suction ports 433 and 543, respectively.

Note that the insertion tool 6 may have a confirmation portion for mechanically confirming an absorption state. Although the confirmation portion is not restricted particularly only if it allows confirmation of the absorption state, for example, it can be configured such that it has a flow range measurement unit (negative pressure gauge) for measuring the flow range from the suction port 543 and a decision unit for deciding whether or not absorption is performed precisely on the basis of a measurement result from the flow range measurement unit.

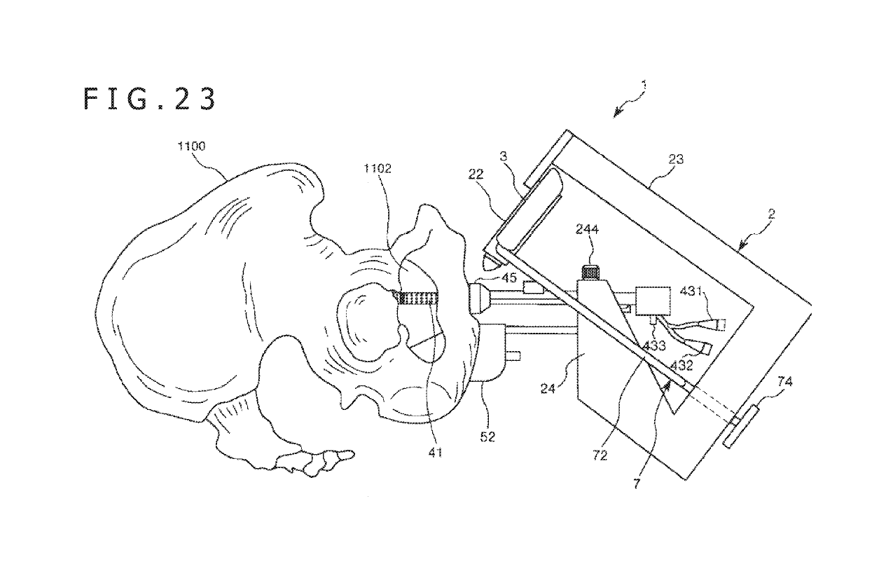

After the urethral wall and the vaginal anterior wall are placed into the state in which they are spaced away from each other sufficiently to allow the puncture member 3 to puncture in such a manner as described above, the frame 2 is fixed to the insertion tool 6 as depicted in FIG. 22B. Consequently, a state in which the puncture apparatus 1 is mounted on the patient is established. In this state, the positional relationship between the pelvis 1100 and the puncture apparatus 1 is such a state as depicted in FIG. 23.

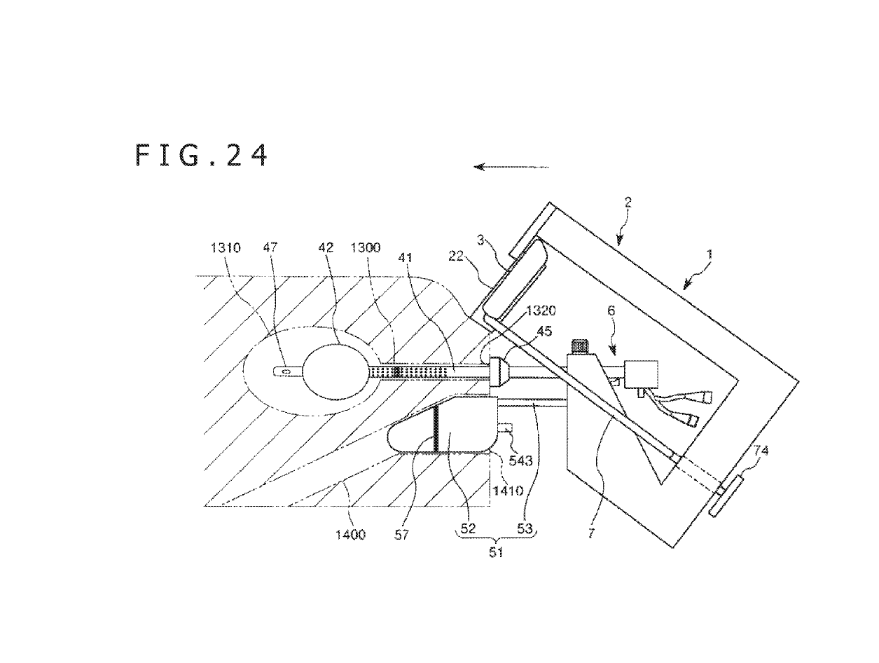

Then, the restriction portion 48 is removed from the urethral-insertion portion 41 as depicted in FIG. 24, and the insertion tool 6 is pushed in to the inner side of the body. Since the urethral wall is absorbed to the urethral-insertion portion 41 and the vaginal wall is absorbed to the vaginal-insertion portion 51 as described hereinabove, when the insertion tool 6 is pushed in to the inner side of the body, the urethra 1300 and the vagina 1400 are pushed in together, whereupon the tissue between them is extended. Therefore, puncture of the tissue by the puncture member 3 can be further facilitated.

In accordance with an exemplary embodiment, for example, in the present embodiment, since the sliding resistance amplification portion 49 for increasing the sliding resistance with respect to the urethral is provided on the urethral-insertion portion 41, the urethra 1300 can be pushed in with a higher degree of certainty. Further, since rubbing (displacement) of the urethral-insertion portion 41 and the urethra 1300 against (from) each other is reduced, damage to the urethra can be reduced.

Note that, at this time, since the restriction portion 48 is removed, the marker 45 is slidably moved toward the proximal side of the urethral-insertion portion 41 while attaching the urethral orifice 1320, and this does not obstruct the pushing in of the urethral-insertion portion 41.

Further, while a case in which both of the urethra 1300 and the vagina 1400 are pushed in to the inner side of the body is described in the foregoing description, only one of the urethra 1300 and the vagina 1400 may be pushed in to the inner side of the body. Also by this, a similar effect can be demonstrated.

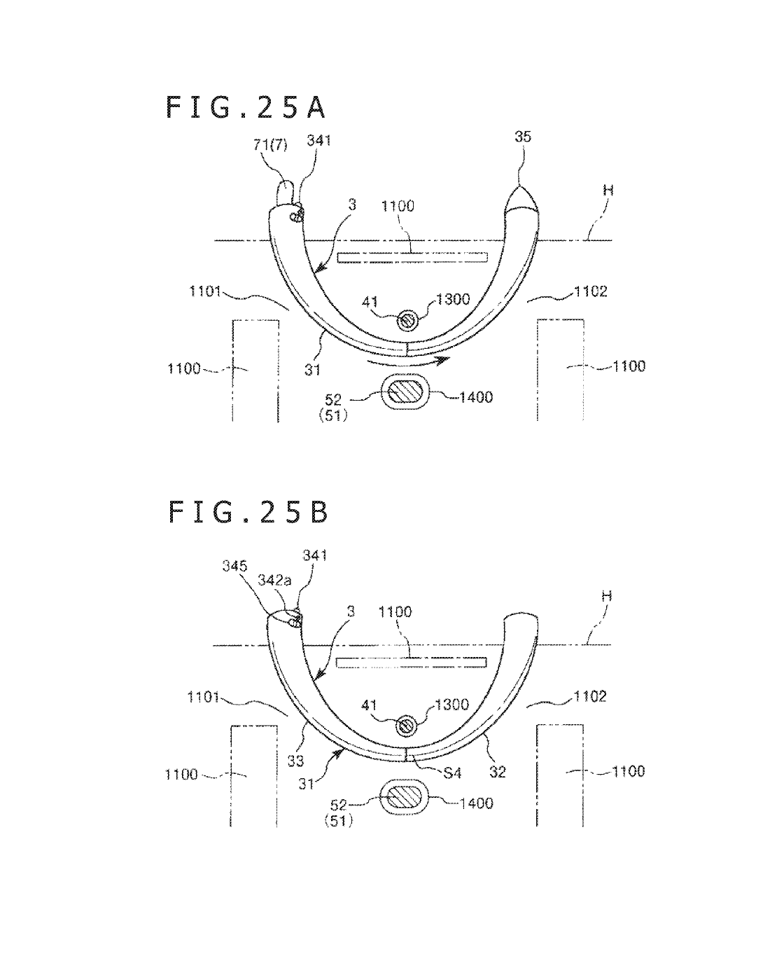

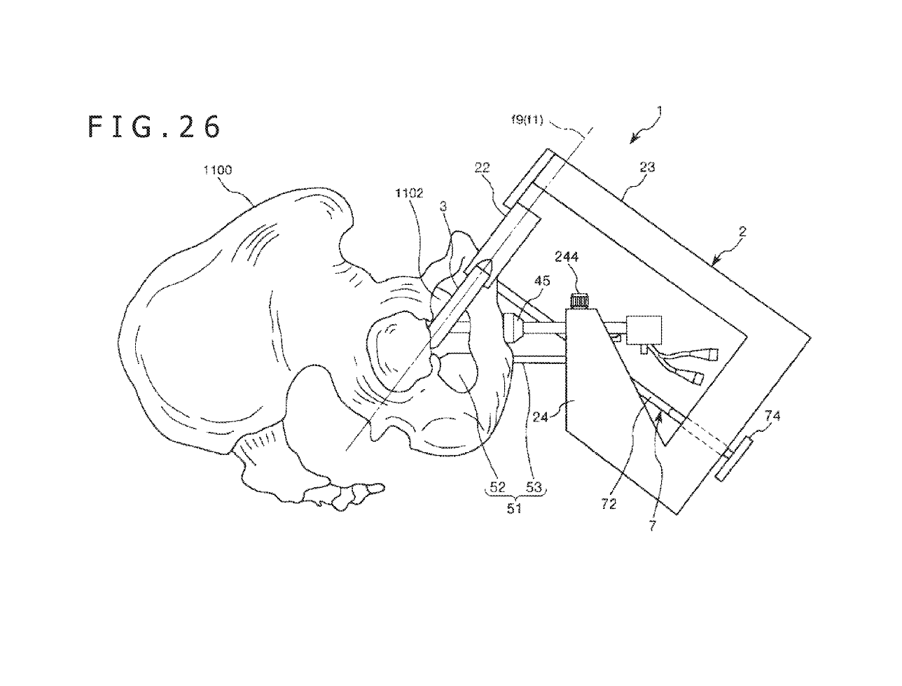

Then, in the state in which the urethra 1300 and the vagina 1400 are pushed in to the inner side of the body, the puncture apparatus 1 is positioned such that the puncture route of the puncture member 3 passes a safety zone S5 of the left and right obturator foramens 1101 and 1102 of the pelvis 1100, and while this state is maintained, the handle 74 is operated to turn the operation member 7 as depicted in FIG. 25A. Consequently, the needle body 35 of the puncture member 3 punctures the body surface H at the right side inguinal region of the patient or in a region in the proximity of the right side inguinal region and enters the body. Then, the needle body 35 successively passes the obturator foramen 1101, a region between the urethra 1300 and the vagina 1400, and the other obturator foramen 1102, and then projects to the outside of the body from the body surface H at the left side inguinal region or in a region in the proximity of the left side inguinal region. Along with this, if the puncture member 3 punctures substantially vertically with respect to the left and right obturator foramens 1101 and 1102 of the pelvis 1100, then the route can be formed at a position suitable for indwelling of the implant 9. In this state, the positional relationship between the pelvis 1100 and the puncture apparatus 1 is such a state as depicted in FIG. 26.

Further, thereupon, the puncture member 3 punctures the living body such that it passes between the marker 46 and the urethral orifice 1320. Consequently, the puncture member 3 punctures a region between a middle portion of the urethra 1300 (middle urethra) and the vagina 1400, and a relatively more effective treatment can be performed.

The puncture member 3 is disposed in the living body thereby. Then, the handle 74 is operated to turn the operation member 7 in the reverse direction to pull out only the operation member 7 from within the living body while the puncture member 3 remains disposed in the living body. Thereafter, the frame 2 is removed from the insertion tool 6, and the needle body 35 is removed from the main body 31 further. Consequently, a state in which only the main body 31 is disposed in the living body is established as depicted in FIG. 25B. The main body 31 is disposed in the living body in the state in which both of the distal side opening and the proximal side opening thereof are exposed to the outside of the living body.

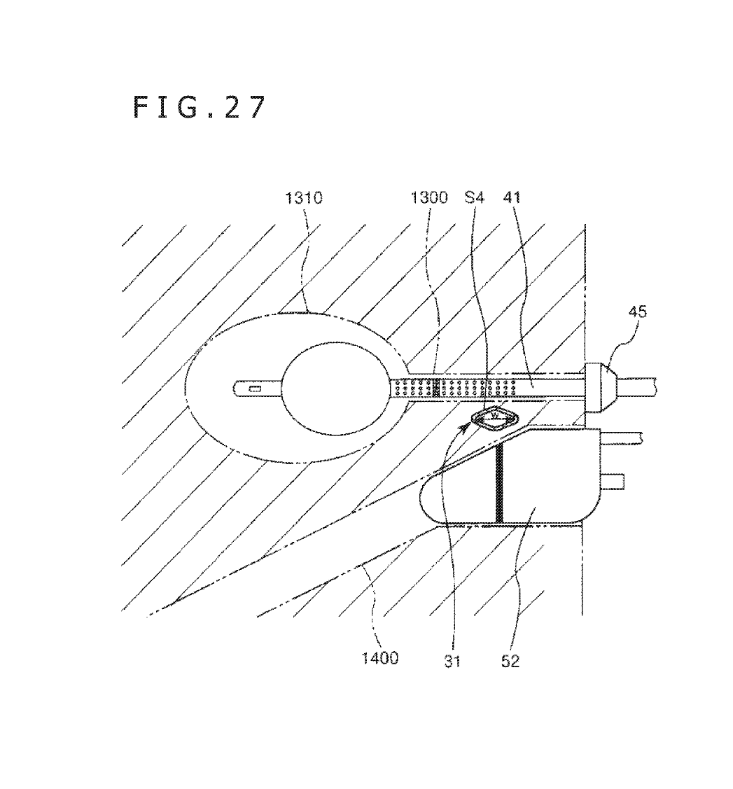

Then, the position of the main body 31 is adjusted as occasion demands. In particular, the left and right projection lengths of the main body 31 can be adjusted to each other to position the central portion S4 of the main body 31 between the urethra 1300 and the vagina 1400. In this state, as depicted in FIG. 27, the widthwise direction (direction of the major axis J32) W of the central portion S4 is disposed substantially in parallel to the urethra 1300. In other words, the urethra 1300 in and by which the urethral-insertion tool 4 is inserted and corrected and the widthwise direction W of the central portion S4 are positioned substantially in parallel to each other.

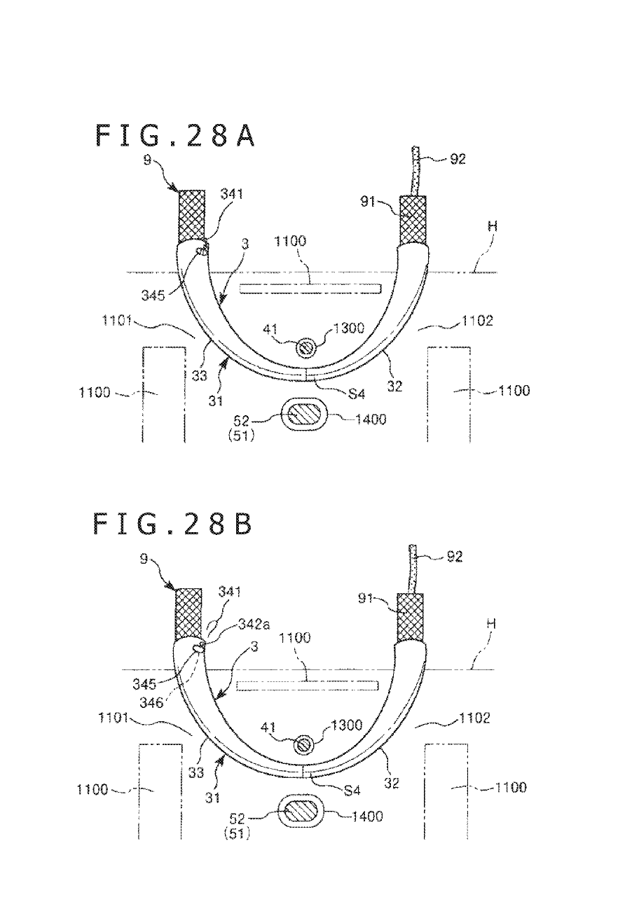

Then, the implant 9 is extracted from the packaging material 90 and inserted into the main body 31 to establish a state in which the implant main body 91 projects from the proximal side opening and the distal side opening of the main body 31 as depicted in FIG. 28A. By keeping the packaging material 90 accommodated until a point of time immediately before it is disposed into the main body 31 in this manner, contamination to the implant 9 can be prevented. Note that, since the main body 31 has a flattened shape as described hereinabove, the posture of the implant main body 91 follows the flattened shape. In particular, the implant main body 91 is disposed into the main body 31 such that the widthwise direction thereof coincides with the widthwise direction of the main body 31. From the relationship to the urethra 1300, the implant main body 91 is disposed in parallel to the corrected urethra 1300.

Then, the string 341 exposed from the exposure holes 345 and 346 is cut as depicted in FIG. 28B. Consequently, the main body 31 is placed into a state in which it can be divided into the distal end divisional piece 32 and the proximal end divisional piece 33. Then, the absorption of the urethral wall by the urethral-insertion portion 41 and the absorption of the vaginal wall by the vaginal-insertion portion 51 can be stopped. Consequently, the position and the shape of the urethra 1300 and the vagina 1400 return to their respective original states.

Then, the connection between the distal end divisional piece 32 and the proximal end divisional piece 33 is cancelled, and the distal end divisional piece 32 is pulled out from the living body toward the distal side and the proximal end divisional piece 33 is pulled out from the living body toward the proximal side. Along with this, the distal end divisional piece 32 and the proximal end divisional piece 33 are moved substantially at the same time in the opposite directions to each other so that the distal end divisional piece 32 and the proximal end divisional piece 33 are moved arcuately along the individual shape. Consequently, the main body 31 is removed smoothly from the living body.

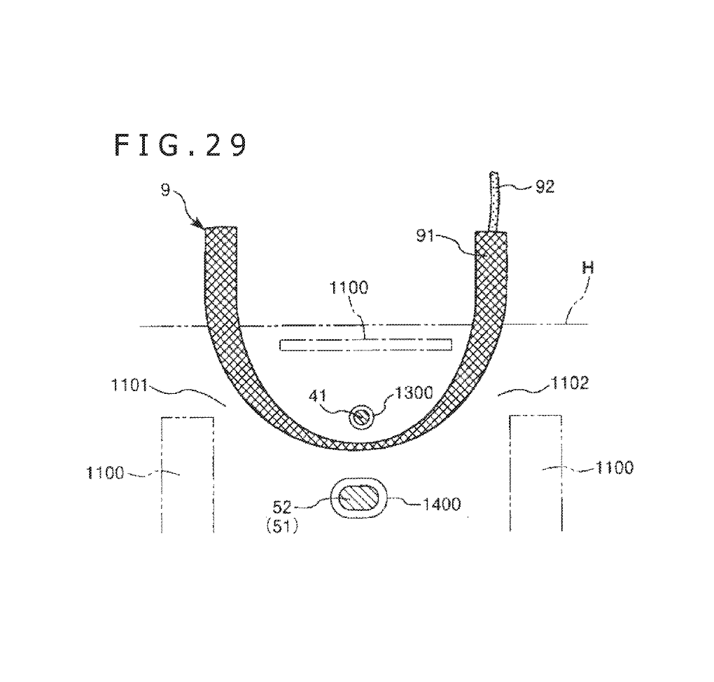

When the distal end divisional piece 32 and the proximal end divisional piece 33 are removed from the living body in such a manner as described above, the surrounding tissue which has been expanded by the main body 31 restores its original position, and the tissue is gradually contacted with the implant main body 91 from a central portion toward the opposite end portions of the implant main body 91. From the fact that the distal end divisional piece 32 and the proximal end divisional piece 33 are moved in a direction along the shape thereof and the fact that the main body 31 has an internal space in which the implant main body 91 can move with sufficiently low friction as described above, the implant main body 91 can be indwelled in its state without being acted upon by unnecessary tensile force. Consequently, the necessity for adjustment of the tension of the implant main body 91 can be eliminated. From the foregoing, a state in which the implant main body 91 is embedded in the living body as depicted in FIG. 29 is established.

In the state in which the implant 9 is embedded in the living body, the implant main body 91 is disposed substantial in parallel to the urethra 1300 in a region between the urethra 1300 and the vagina 1400. Therefore, the urethra 1300 can be supported over a wider area by the implant main body 91.

By dividing and removing the main body 31 from the living body in this manner, removal of the main body 31 from the living body can be performed relatively easily. Further, since the divisional pieces 32 and 33 during the removal little have an influence on the posture of the implant main body 91 in the region between the urethra 1300 and the vagina 1400, the implant main body 91 can be embedded in a desired posture.

Further, since the divisional pieces 32 and 33 are removed from the living body in the state in which the urethral-insertion portion 41 is inserted in the urethra 1300, excessive tension can be prevented from being applied to the urethra 1300 by the implant main body 91 to be indwelled in the living body.

Then, the insertion tool 6 is removed from the living body. In particular, the urethral-insertion portion 41 is removed from the urethra 1300 and the vaginal-insertion portion 51 is removed from the vagina 1400. After the urethral-insertion tool 4 is removed, the urethra 1300 returns to a shape of a natural state. However, since the implant main body 91 is embedded in the living body tissue, the urethra 1300 in its natural state and the implant main body 91 can be maintained in a parallel state.

Thereafter, any unnecessary portion of the implant main body 91 can be cut away, and the procedure is ended.

As described above, with the puncture apparatus 1, when the implant 9 is to be indwelled, this can be coped with only by a procedure of minimal invasion such as puncture by the puncture member 3 and incision or the like which is large in invasion need not be performed. Therefore, the burden on the patient is relatively low and the safety of the patient is relatively high as well. Further, since the implant main body 91 can be embedded in parallel to the urethra 1300, the urethra 1300 can be supported over a wider area. Further, the living body can be punctured by the puncture member 3 avoiding the urethra 1300 and the vagina 1400, and can help prevent the puncture member 3 from puncturing the urethra 1300 or the vagina 1400. Further, it can help prevent the implant 9 from being exposed to the inside of the vagina from a wound caused by incision of the vagina as in a conventional case in which the vagina is incised or that occurrence of complications such as infection from the wound. The puncture apparatus 1 is relatively safe and can embed the implant 9 with relative certainty.

FIG. 30 is a lateral view depicting an insertion tool, which includes a puncture apparatus according to a second embodiment.

Although the second embodiment of the puncture apparatus is described below with reference to FIG. 30, the description is given principally of differences of the second embodiment from the aforementioned first embodiment while description of like matters are omitted herein to avoid redundancy.

The present embodiment is similar to the aforementioned first embodiment except that it is different in configuration of the urethral-insertion tool.

As depicted in FIG. 30, in the urethral-insertion tool 4 of the present embodiment, the marker 45 is provided on the front face of the urethral-insertion portion 41. Further, the marker 45 does not substantially project from the front face of the urethral-insertion portion 41. Therefore, the marker 45 can advance into the urethra 1300 together with the urethral-insertion portion 41. Further, the marker 45 can be fixedly applied to the front face of the urethral-insertion portion 41 and cannot move relative to the urethral-insertion portion 41. Such a marker 45 as described above does not have a function as the inflow reduction portion 450.

By configuring the marker 45 in such a manner as described above, the configuration of the puncture apparatus 1 can be simplified in comparison, for example, with the first embodiment. Further, an operation of the puncture apparatus 1 at a predetermined stage is facilitated. In particular, for example, in the aforementioned first embodiment, the marker 45 projects from the urethral-insertion portion 41 and cannot advance into the urethra 1300, and therefore, in order to establish the state depicted in FIG. 24, the restriction portion 48 must be removed to place the marker 45 into a slidably movable state with respect to the urethral-insertion portion 41. In contrast, in the present embodiment, since the marker 45 can advance into the urethra 1300, such an operation as described above is not required, and the operation can be performed smoothly as much.

The configuration of the marker 45 is not restricted particularly only if the operator can visually observe the same from the outside. For example, the marker 45 may be configured from a colored portion colored in a different color from the surroundings or may be configured from a recess or a projection.

Note that, while, in the present embodiment, the marker 45 does not substantially project from the outer periphery of the urethral-insertion portion 41, it may project a little from the outer periphery of the urethral-insertion portion 41 only if it can advance into the urethra 1300.

Further, in the urethral-insertion tool 4 of the present embodiment, the sliding resistance amplification portion 49 is configured from a ring-shaped protrusion (projection) projecting from the outer periphery of the urethral-insertion portion 41. Also by configuring the sliding resistance amplification portion 49 in this manner, the sliding resistance of the urethral-insertion portion 41 with respect to the urethra 1300 can be effectively increased. Note that, while, in the present embodiment, a single ring-shaped projection is provided, the number of such projections is not limited particularly. For example, a plurality of projections may be provided in a juxtaposed relationship in the lengthwise direction of the urethral-insertion portion 41. Further, the projection may not have a ring shape, and such a configuration that a plurality of wart-like projections are disposed along a circumferential direction of the urethral-insertion portion 41 may be adopted or the projection may be a spiral projection extending in the longitudinal direction of the urethral-insertion portion 41.

Furthermore, in the present embodiment, the sliding resistance amplification portion 49 can also serve as the marker 46. Therefore, the configuration of the urethral-insertion tool 4 can be simplified. Note that, in order that the function as the marker 46 may be demonstrated more readily, the sliding resistance amplification portion 49 is preferably colored in a color different from that of the surroundings (distal side and proximal side).

Also with such a second embodiment as described above, effects similar to those of the aforementioned first embodiment can be demonstrated.

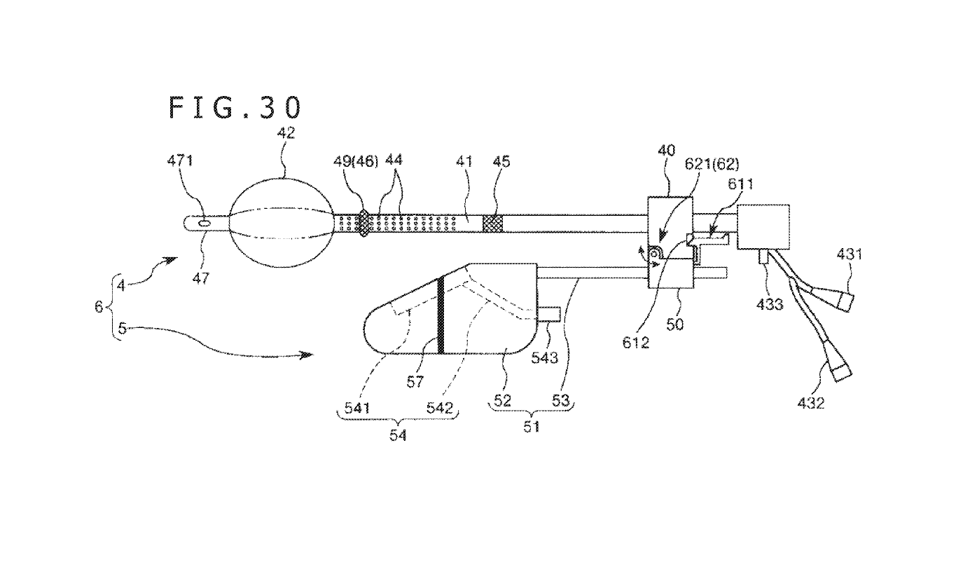

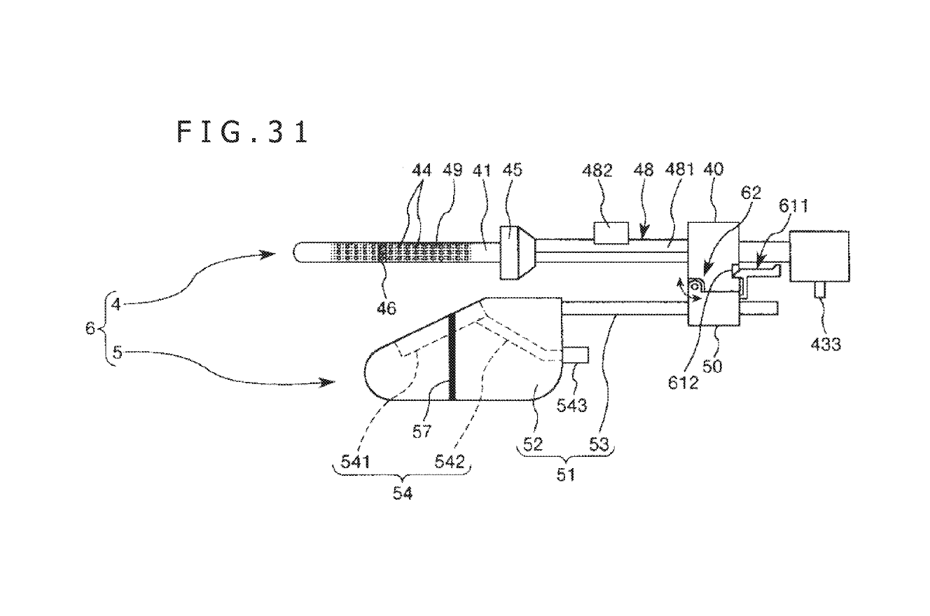

FIG. 31 is a lateral view depicting an insertion tool, which includes a puncture apparatus according to a third embodiment.

Although the third embodiment of the puncture apparatus is described below with reference to FIG. 31, the description is given principally of differences of the third embodiment from the aforementioned first embodiment while description of like matters are omitted herein to avoid redundancy.

The present embodiment is similar to the aforementioned first embodiment except that it is different in configuration of the urethral-insertion tool.

As depicted in FIG. 31, in the urethral-insertion tool 4 of the present embodiment, the balloon 42 and the urine drainage portion 47 are omitted. With such a configuration as just described, the configuration of the urethral-insertion tool 4 can be simplified. Note that, in the puncture apparatus 1, even if the balloon 42 used to be engaged principally with the bladder neck to perform positioning of the urethral-insertion portion 41 in order to position the puncture position of the puncture member 3 with respect to the urethral orifice 1320 is omitted, the operability of the puncture apparatus 1 upon puncture by the puncture member 3 deteriorates relatively little.

Also with such a third embodiment as described above, effects similar to those of the aforementioned first embodiment can be demonstrated.

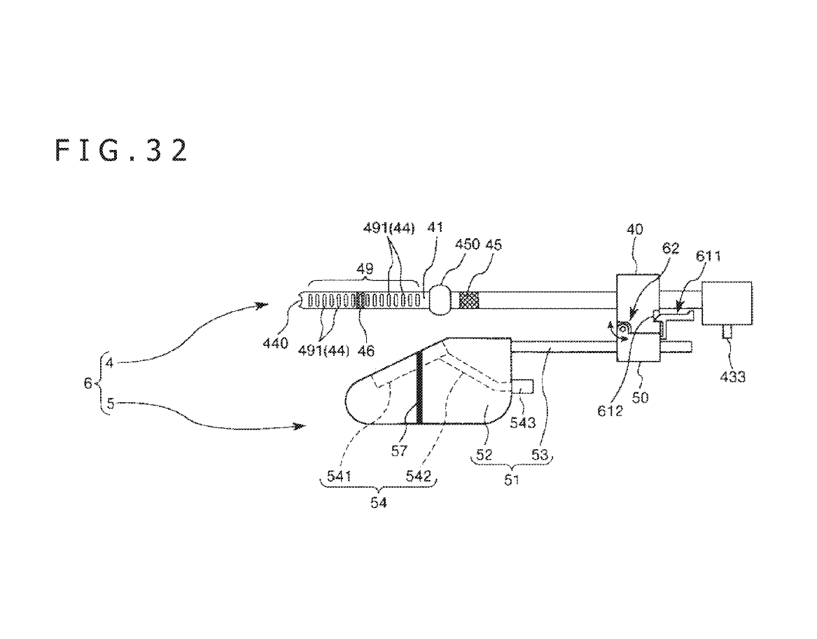

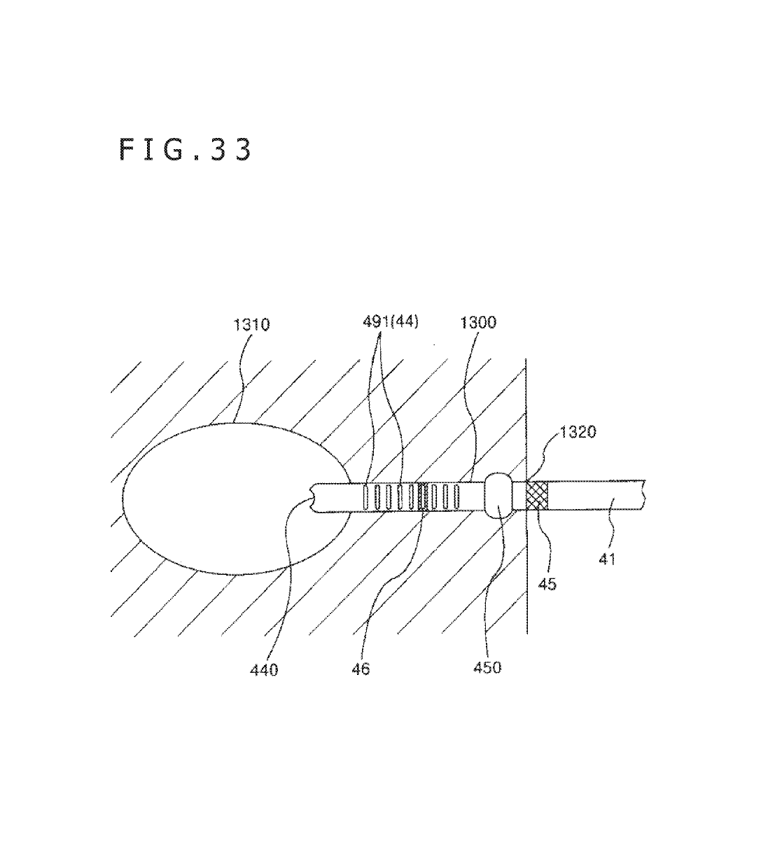

FIG. 32 is a lateral view depicting an insertion tool, which includes a puncture apparatus according to a fourth embodiment. FIG. 33 is a sectional view depicting the urethral-insertion tool, which includes the insertion tool depicted in FIG. 32 in a state in which it is inserted in the urethra.

Although the fourth embodiment of the puncture apparatus is described below with reference to FIGS. 32 and 33, the description is given principally of differences of the fourth embodiment from the aforementioned embodiments while description of like matters are omitted herein to avoid redundancy.

The present embodiment is similar to the aforementioned first embodiment except that it is different in configuration of the urethral-insertion tool.

As depicted in FIG. 32, in the urethral-insertion tool 4 of the present embodiment, the balloon 42 and the urine drainage portion 47 are omitted.

Further, in the urethral-insertion tool 4 of the present embodiment, the marker 45 is provided on the front face of the urethral-insertion portion 41 but does not substantially project from the front face of the urethral-insertion portion 41. Therefore, the marker 45 can advance into the urethra 1300. Further, the marker 45 can be fixedly applied to the front face of the urethral-insertion portion 41 and cannot move relative to the urethral-insertion portion 41. Such a configuration of the marker 45 as just described is similar to that of the aforementioned second embodiment.

With such a marker 45 as just described, since it cannot serve also as the inflow reduction portion 450 as in the case of the aforementioned first embodiment, in the present embodiment, the inflow reduction portion 450 is provided separately from the marker 45.

The inflow reduction portion 450 is provided between the marker 45 and the region 51, and in a state in which the urethral-insertion portion 41 is inserted in the urethra 1300 and the marker 45 is positioned at the urethral orifice 1320, the marker 45 is positioned in the urethra 1300. Further, the inflow reduction portion 450 is configured from a ring-shaped projection (protrusion) provided projecting from the outer periphery of the urethral-insertion portion 41.

Further, the inflow reduction portion 450 has an outer diameter gradually decreasing from a central portion toward the opposite ends (distal end and proximal end) thereof, and the outer diameter at the opposite ends is substantially equal to the outer diameter of the urethral-insertion portion 41. Therefore, a step is not substantially formed between the inflow reduction portion 450 and the urethral-insertion portion 41, and the urethral-insertion portion 41 can be inserted in lower invasion into the urethra 1300.

In a state in which the urethral-insertion portion 41 is inserted in the urethra 1300 as depicted in FIG. 33, the inflow reduction portion 450 is positioned in the urethra 1300. Since the inflow reduction portion 450 has an outer diameter greater than that of any other portion of the urethral-insertion portion 41 (portions on the distal side and the proximal side of the inflow reduction portion 450), the urethral wall contacts more closely at the inflow reduction portion 450 than at any other portion of the urethral-insertion portion 41. Therefore, at the inflow reduction portion 450, the urethra 1300 can be sealed, and inflow of air to the interior side than the inflow reduction portion 450 of the urethra 1300 from the urethral orifice 1320, namely, to the location at which the suction holes 44 is positioned, can be reduced. As a result, absorption of the urethral wall by the suction holes 44 can be performed with a relatively higher degree of certainty and effectiveness.

Further, in the urethral-insertion tool 4 of the present embodiment, the sliding resistance amplification portion 49 is configured such that it has a groove 491, which extends in a circumferential direction of the urethral-insertion portion 41. Actually, a plurality of such grooves 491 are provided along the longitudinal direction of the urethral-insertion portion 41. Note that the number of grooves 491 disposed along the circumferential direction of the urethral-insertion portion 41 is not restricted particularly, and may be one or two or more. Also with such a sliding resistance amplification portion 49 as just described, the sliding resistance of the urethral-insertion portion 41 with respect to the urethra 1300 can be increased by a simple configuration.

In accordance with an exemplary embodiment, for example, since each groove 491 is formed as a groove extending in the circumferential direction of the urethral-insertion portion 41, the sliding resistance with respect to the urethra 1300 when the urethral-insertion portion 41 is pushed in to the inner side of the body can be increased in comparison with that in an alternative case in which the groove 491 is formed as a circular groove or a groove extending in the longitudinal direction of the urethral-insertion portion 41, for example. Further, also by providing a plurality of grooves 491 in the longitudinal direction of the urethral-insertion portion 41, the sliding resistance with respect to the urethra 1300 when the urethral-insertion portion 41 is pushed in to the inner side of the body can be increased. Therefore, with the present embodiment, the aforesaid effects can be further enhanced.

Further, in the present embodiment, at least one of the grooves 491 serves also as a suction hole 44. In particular, for example, the groove 491 which serves also as the suction hole 44 passes the inside of the urethral-insertion portion 41 and is connected to the suction port 433. By such a configuration as just described, the configuration of the urethral-insertion tool 4 can be simplified further. Further, where the groove 491 also serves as the suction hole 44, catching between the urethral wall and the groove 491 in a state in which the urethral wall is absorbed is strengthened. Therefore, the sliding resistance of the urethral-insertion portion 41 with respect to the urethra can be further increased.

Further, the urethral-insertion tool 4 of the present embodiment has a suction hole 440, which faces the distal end of the urethral-insertion portion 41. As depicted in FIG. 33, the suction hole 440 is positioned in the bladder 1310 when the urethral-insertion portion 41 is inserted in the urethra 1300 until the marker 45 is positioned at the urethral orifice 1320. Therefore, the bladder wall can be absorbed by the suction hole 440. Consequently, the bladder 1310 can be pushed in to the inner side of the body with a higher degree of effectiveness together with the urethral-insertion portion 41. Note that the suction hole 440 may also serve as the urine drainage portion 47 for draining the urine in the bladder 1310.

Also with such a fourth embodiment as described above, effects similar to those of the aforementioned first embodiment can be demonstrated.

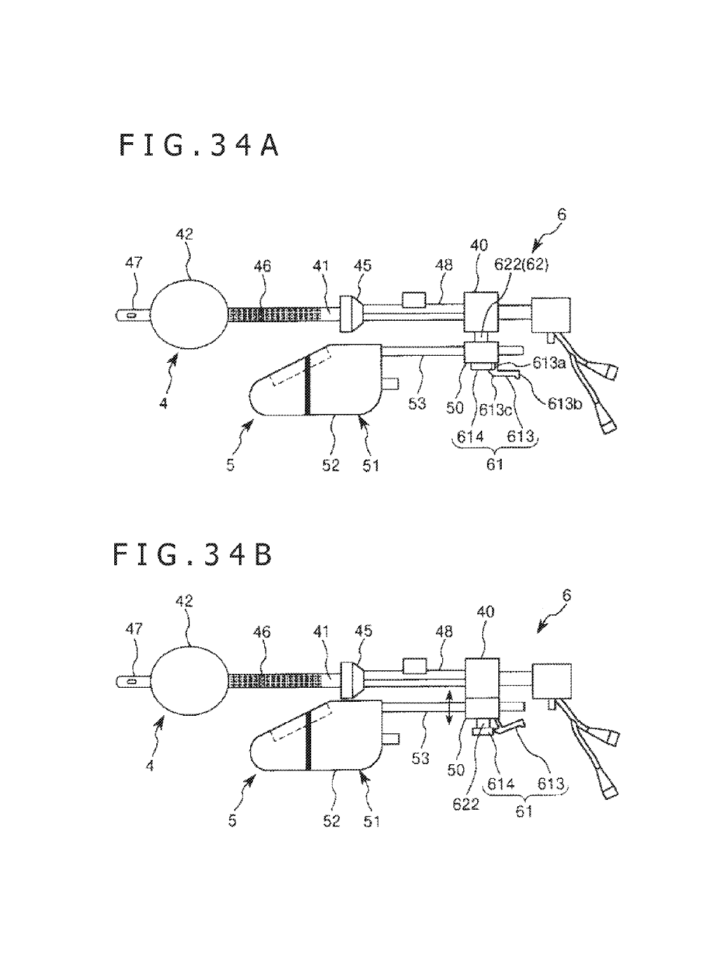

FIGS. 34A and 34B depict an insertion tool, which includes a puncture apparatus according to a fifth embodiment, and wherein FIG. 34A is a view depicting the insertion tool in a state in which it is locked by a maintaining portion and FIG. 34B is a view depicting the insertion tool in another state in which it is not locked by the maintaining portion.

Although the fifth embodiment of the puncture apparatus is described below with reference to FIGS. 34A and 34B, the description is given principally of differences of the fifth embodiment from the aforementioned embodiments while description of like matters are omitted herein to avoid redundancy.

The present embodiment is similar to the aforementioned first embodiment except that it is different in configuration of the urethral-insertion tool, particularly in configuration of the displacement portion and the maintaining portion.

As depicted in FIG. 34A, the displacement portion 62 which the insertion tool 6 of the present embodiment has interlocks the urethral-insertion portion 41 and the vaginal-insertion portion 51 for movement toward and away from each other. In accordance with an exemplary embodiment, for example, the displacement portion 62 has a slide shaft 622 projecting to the lower side from the supporting portion 40, and the supporting portion 50 is interlocked for sliding movement to the slide shaft 622. Note that the supporting portion 50 is provided against rotation with respect to the slide shaft 622. Further, a flange (engaging portion) 614 is provided at a distal portion of the slide shaft 622 such that removal of the supporting portion 50 from the slide shaft 622 is prevented by the flange 614. By providing the vaginal-insertion tool 5 for sliding movement with respect to the slide shaft 622 in this manner, the urethral-insertion portion 41 and the vaginal-insertion portion 51 can be moved toward and away from each other by a relatively simple operation.

Meanwhile, the maintaining portion 61 can assume a state in which the urethral-insertion tool 4 and the vaginal-insertion tool 5 maintain their relative positional relationship and another state in which the vaginal-insertion tool 5 is slidably movable with respect to the urethral-insertion tool 4. Such a maintaining portion 61 as just described has the aforementioned flange 614, and an operation portion 613 provided at the supporting portion 50. Further, the operation portion 613 has a shaft portion 613a which can be flexion-deformed, a knob 613b interlocked to the shaft portion 613a, and a pawl portion 613c provided at a distal portion of the knob 613b. The pawl portion 613c is engageable with the flange 614.

By placing the pawl portion 613c into a state in which it engages with the flange 614 as depicted in FIG. 34A, the supporting portion 40 and the supporting portion 50 are fixed to each other, and the relative positional relationship between the urethral-insertion tool 4 and the vaginal-insertion tool 5 can be maintained. Meanwhile, by pulling the knob 613b upwardly to remove the pawl portion 613c from the flange 614 as depicted in FIG. 34B, the lock (maintaining state) is cancelled to place the vaginal-insertion tool 5 into a state in which it can slidably move with respect to the urethral-insertion tool 4. Further, in the state in which vaginal-insertion tool 5 is slidably movable, the distal portion 52 (vaginal-insertion portion 51) can be positioned more closely to the urethral-insertion portion 41 than that in the maintaining state depicted in FIG. 34A.

Note that, while, in the present embodiment, the slide shaft 622 is provided on the supporting portion 40 and the operation portion 613 is provided on the supporting portion 50, conversely the slide shaft 622 may be provided on the supporting portion 50 while the operation portion 613 is provided on the supporting portion 40. Further, the configuration of the operation portion 613 is not limited to that of the present embodiment only if it can demonstrate similar effects.

Also with such a fifth embodiment as described above, effects similar to those of the aforementioned first embodiment can be demonstrated.

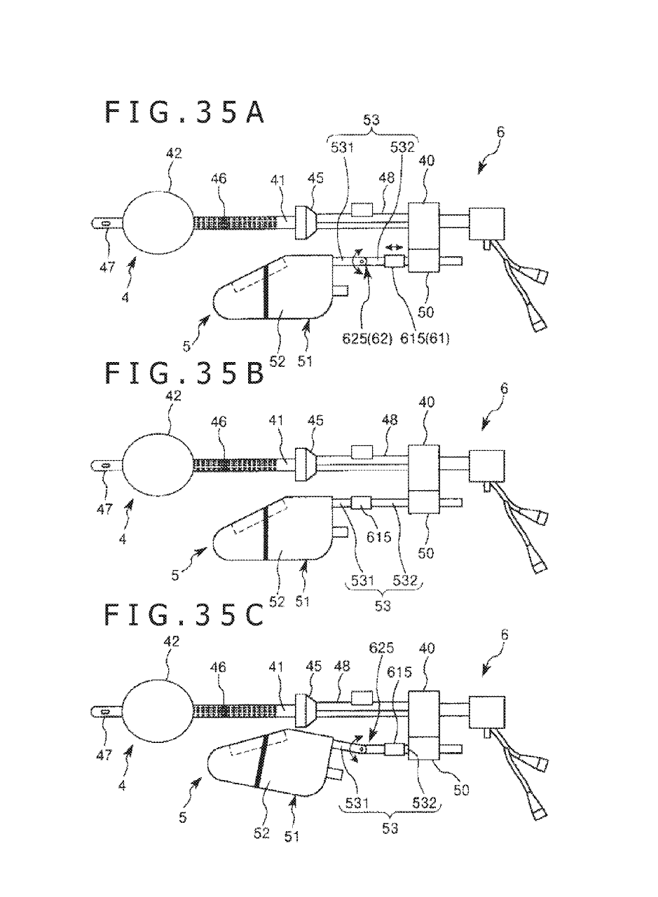

FIG. 35A is a lateral view of an insertion tool, which includes a puncture apparatus according to a sixth embodiment. FIG. 35B is a lateral view depicting the insertion tool in a state in which it is locked by a maintaining portion, and FIG. 35C is a lateral view depicting the insertion tool in another state in which it is not locked by the maintaining portion.

Although the sixth embodiment of the puncture apparatus is described below with reference to FIGS. 35A to 35C, the description is given principally of differences of the sixth embodiment from the aforementioned embodiments while description of like matters are omitted herein to avoid redundancy.

The present embodiment is similar to the aforementioned first embodiment except that it is different in configuration of the urethral-insertion tool, particularly in configuration of the displacement portion and the maintaining portion.

As depicted in FIG. 35A, in the insertion tool 6 of the present embodiment, the supporting portions 40 and 50 are fixed.

Further, in the insertion tool 6 of the present embodiment, the shaft portion 53 of the vaginal-insertion portion 51 can be flexion-deformed midway thereof by the displacement portion 62. In particular, for example, the shaft portion 53 is configured from two divisional parts including a distal side shaft portion 531 positioned on the distal side and a proximal side shaft portion 532 positioned on the proximal side. The distal side shaft portion 531 and the proximal side shaft portion 532 are interlocked for rotary movement to each other by a hinge portion 625. Thus, by moving rotationally the distal side shaft portion 531 with respect to the proximal side shaft portion 532, the distal portion 52 can be moved toward and away from the urethral-insertion portion 41.

Meanwhile, the maintaining portion 61 can assume a state in which the distal side shaft portion 531 and the proximal side shaft portion 532 are maintained in a linear posture and another state in which the distal side shaft portion 531 can be moved rotationally with respect to the proximal side shaft portion 532. Such a maintaining portion 61 as just described has a tubular slider (operation portion) 615 provided on an outer circumference of the shaft portion 53 for sliding movement in the longitudinal direction with respect to the shaft portion 53.

By placing the slider 615 into a state in which it is covered with the hinge portion 625 as depicted in FIG. 35B, the distal side shaft portion 531 and the proximal side shaft portion 532 are maintained in a linear posture and the relative positional relationship of the urethral-insertion portion 41 and the distal portion 52 is maintained. On the other hand, by slidably moving the slider 615 into a state in which the hinge portion 625 is exposed as depicted in FIG. 35C, the lock (maintained state) is cancelled and the distal portion 52 is placed into a state in which it can move rotationally with respect to the urethral-insertion portion 41. In the rotary movement state, the distal portion 52 can be positioned more closely to the urethral-insertion portion 41 than in the maintained state depicted in FIG. 35B. With such a maintaining portion 61 as just described, an operation thereof can be performed.

Also with such a sixth embodiment as described above, effects similar to those of the aforementioned first embodiment can be demonstrated.

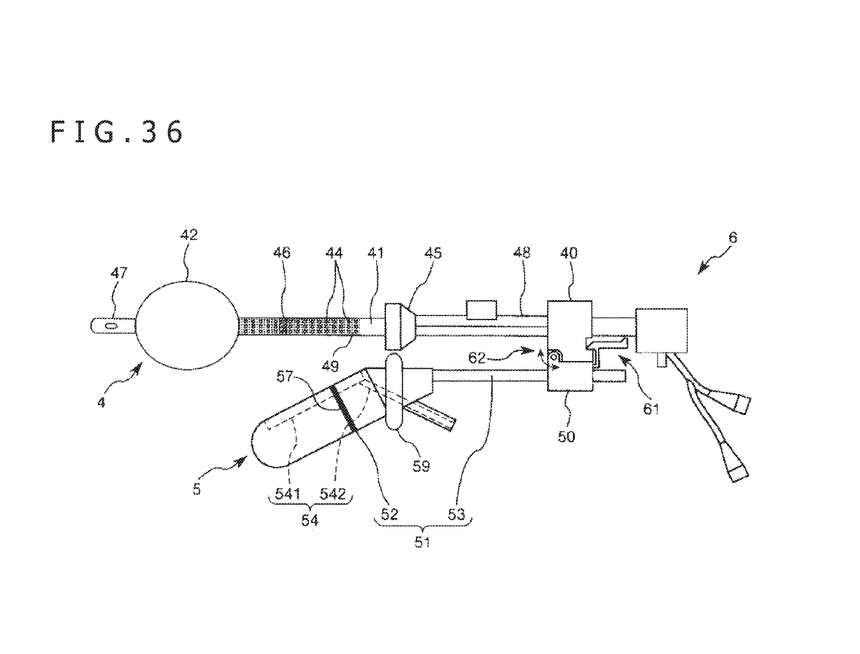

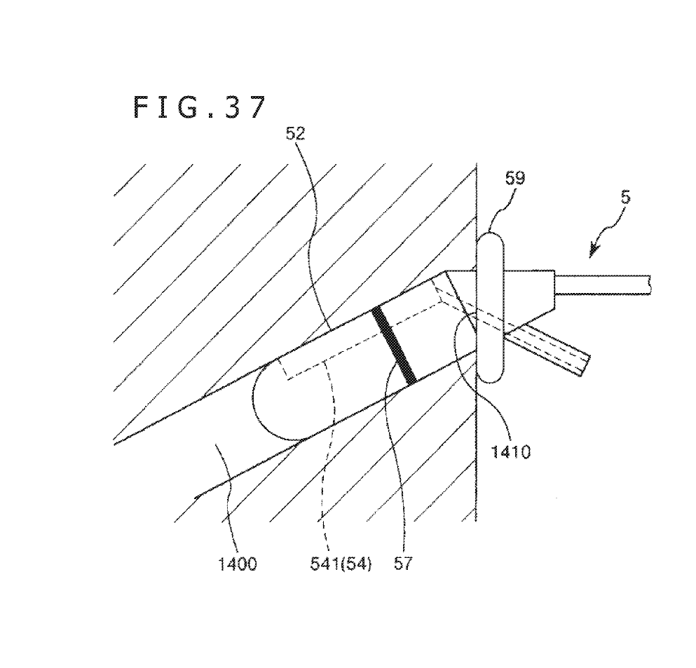

FIG. 36 is a lateral view of an insertion tool, which includes a puncture apparatus according to a seventh embodiment. FIG. 37 is a sectional view depicting a vaginal-insertion tool depicted in FIG. 36 in a state in which it is inserted in the vagina.

Although the seventh embodiment of the puncture apparatus is described below with reference to FIGS. 36 and 37, the description is given principally of differences of the seventh embodiment from the aforementioned embodiments while description of like matters are omitted herein to avoid redundancy.

The present embodiment is similar to the aforementioned first embodiment except that it is different in configuration of the vaginal-insertion tool.

As depicted in FIG. 36, in the vaginal-insertion tool 5 of the present embodiment, the distal portion 52 has a flattened shape crushed in the heightwise direction. Therefore, the distal portion 52 can be inserted readily into the vagina, for example, in comparison with that of the aforementioned first embodiment.

Further, a ring-shaped inflow reduction portion 59 is provided at a proximal portion of the distal portion 52, more particularly, on the proximal side with respect to the recessed portion 541, such that is projects from an outer periphery of the distal portion 52. The inflow reduction portion 59 can be configured from such a member as, for example, an O-ring. Such an inflow reduction portion 59 as just described is not inserted in the vagina 1400 together with the distal portion 52. Furthermore, the inflow reduction portion 59 projects sufficiently such that it covers the vaginal orifice 1410. Further, the inflow reduction portion 59 preferably has air tightness and flexibility and can be configured, for example, from a rubber material.

As depicted in FIG. 37, such an inflow reduction portion 59 as described above attaches to the vaginal orifice 1410 in a state in which the distal portion 52 is inserted in the vagina 1400 to cover and close up the vaginal orifice 1410 to reduce inflow of air into the vagina 1400 through the vaginal orifice 1410. Consequently, absorption of the vaginal wall by the suction hole 54 can be performed with a relatively higher degree of certainty and effectiveness.

Note that the inflow reduction portion 59 may be fixed to the distal portion 52 or may be slidably movable in a longitudinal direction on the distal portion 52. Where the inflow reduction portion 59 is slidably movable in the longitudinal direction on the distal portion 52, since the position of the inflow reduction portion 59 can be adjusted, the aforesaid effects can be demonstrated more remarkably.

Further, preferably the inflow reduction portion 59 is freely detachable from the distal portion 52, which makes it possible to demonstrate the aforesaid effects more remarkably by preparing a plurality of inflow reduction portions 59, for example, having shapes different from each other and selectively mounting one of the inflow reduction portions 59 suitable for the patient on the distal portion 52.

Also with such a seventh embodiment as described above, effects similar to those of the aforementioned first embodiment can be demonstrated.

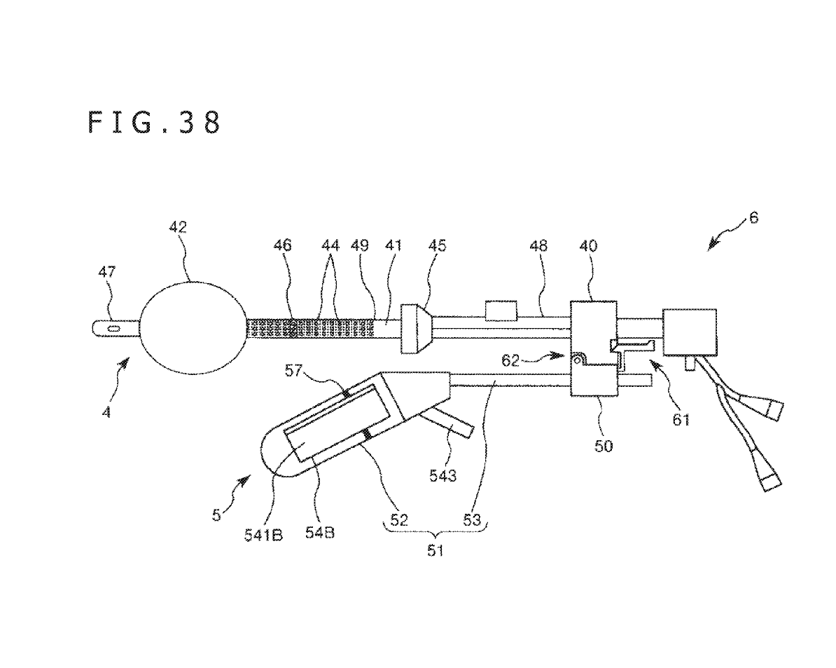

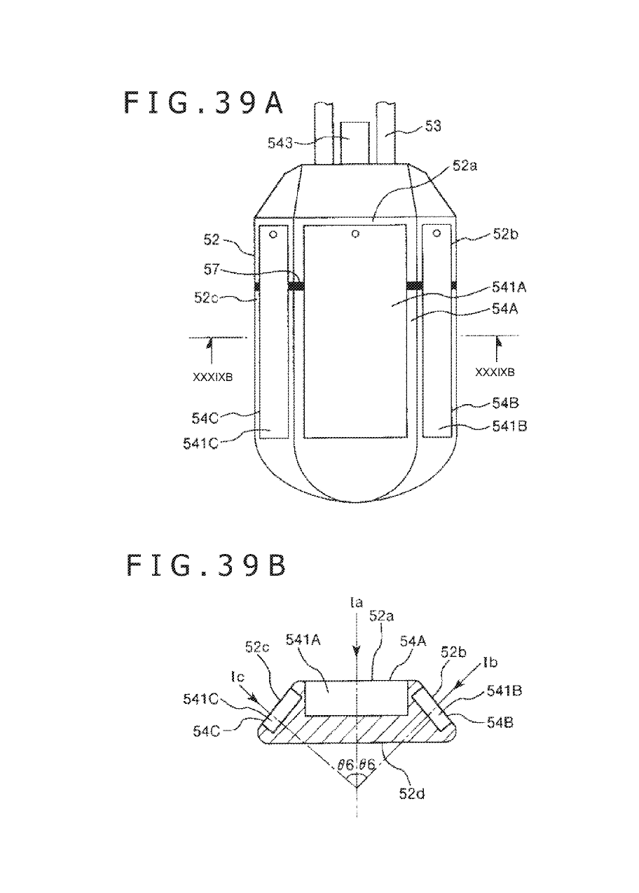

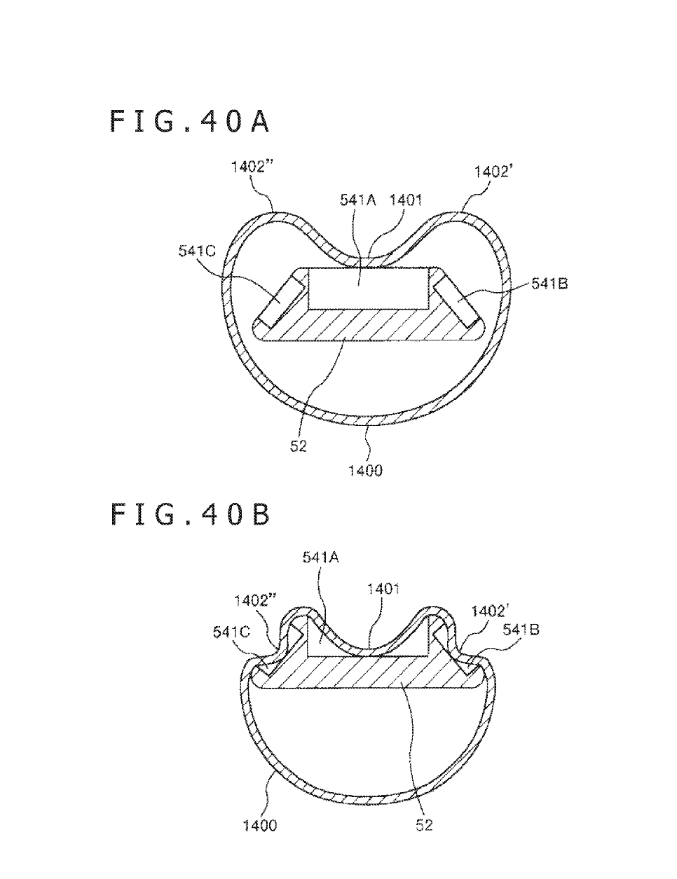

FIG. 38 is a lateral view of an insertion tool, which includes a puncture apparatus according to an eighth embodiment. FIGS. 39A and 39B depict a distal portion of a vaginal-insertion tool depicted in FIG. 38, and wherein FIG. 39A is a top plan view and FIG. 39B is a sectional view taken along line XXXVIIIB-XXXVIIIB in FIG. 39A. FIGS. 40A and 40B depict the distal portion depicted in FIGS. 39A and 39B in a state in which it is inserted in the vagina, and wherein FIG. 40A is a view depicting the distal portion in a state before the vaginal wall is absorbed and FIG. 40B is a view depicting the distal portion in another state in which the vaginal wall is absorbed.

Although the eighth embodiment of the puncture apparatus is described below with reference to FIGS. 38 to 40B, the description is given principally of differences of the eighth embodiment from the aforementioned embodiments while description of like matters are omitted herein to avoid redundancy.

The present embodiment is similar to the aforementioned first embodiment except that it is different in configuration of the vaginal-insertion tool.

As depicted in FIG. 38, in the vaginal-insertion tool 5 of the present embodiment, the distal portion 52 has a flattened shape crushed in the heightwise direction. Therefore, the distal portion 52 can be inserted into the vagina readily, for example, in comparison with that of the aforementioned first embodiment. Further, as depicted in FIGS. 39A and 39B, the distal portion 52 has an upper face (first face) 52a and a lower face 52d disposed in an opposing relationship to each other, and side faces (second faces) 52b and 52c which interlock the upper face 52a and the lower face 52d to each other and are inclined with respect to the upper face 52a. The distal portion 52 has a substantially trapezoidal transverse sectional shape.

Further, the distal portion 52 has a suction hole (first suction hole) 54A open to the upper face 52a, another suction hole (second suction hole) 54B open to the side face 52b, and a further suction hole (second suction hole) 54C open to the side face 52c. In other words, the distal portion 52 of the present embodiment is configured such that a pair of suction holes 54B and 54C are disposed on the opposite sides of the suction hole 54A. The three suction holes 54A, 54B and 54C are connected to the suction port 543 individually through the inside of the distal portion 52.

Further, the suction hole 54A has a recessed portion 541A open over a wide range to the upper face 52a, the suction hole 54B has another recessed portion 541B open over a wide range to the side face 52b, and the suction hole 54C has a further recessed portion 541C open over a wide range to the side face 52c. Further, the recessed portion 541A has a depth greater than that of the recessed portions 541B and 541C and has a width greater than that of the recessed portions 541B and 541C.

Further, the three suction holes 54A, 54B and 54C are different in suction direction from each other. The suction hole 54A has a suction direction (first suction direction) la along a normal to the upper face 52a, the suction hole 54B has a suction direction (second suction direction) 1b along a normal to the side face 52b, and the suction hole 54C has a suction direction (second suction direction) 1c along a normal to the side face 52c. By adopting such a configuration as just described, absorption of the vagina 1400, which has such a depressed portion 1402 as described with reference to aforementioned FIG. 17, can be performed more efficiently.

Describing more particularly, in a state in which the distal portion 52 is inserted in the vagina 1400 as depicted in FIG. 40A, the recessed portion 541A is directed to a central portion 1401 (depending portion) of the vaginal anterior wall and the recessed portion 541B is directed to a depressed portion 1402′ neighboring with the central portion 1401 while the recessed portion 541C is directed to a depressed portion 1402″ neighboring with the central portion 1401. Therefore, if absorption of the vaginal wall by the suction holes 54A, 54B and 54C is performed, then the central portion 1401 is absorbed to the recessed portion 541A, the depressed portion 1402′ (1402) is absorbed to the recessed portion 541B, and the depressed portion 1402″ (1402) is absorbed to the recessed portion 541C as depicted in FIG. 40B. In this manner, with the distal portion 52 of the present embodiment, not only the central portion 1401 but also the depressed portions 1402′ and 1402″ can be absorbed, and the vaginal anterior wall can be absorbed over a substantially overall area thereof with a relatively high degree of certainty.

In accordance with an exemplary embodiment, for example, by forming the recessed portion 541A such that it has a depth greater than that of the recessed portions 541B and 541C and has a width greater than that of the recessed portions 541B and 541C, the central portion 1401 can be absorbed to a greater depth. Therefore, the depressed portions 1402′ and 1402″ on the opposite sides of the central portion 1401 follow the central portion 1401 to approach the recessed portions 541B and 541C, respectively. As a result, the depressed portions 1402′ and 1402″ can be absorbed with a relative higher degree of certainty by the recessed portions 541B and 541C, respectively.

Here, although an angle θ6 defined by the suction direction 1a of the suction hole 54A and the suction direction 1b of the suction hole 54B are not restricted particularly, it preferably is, for example, approximately 20 degrees to 90 degrees, and more preferably is approximately 30 degrees to 50 degrees. Consequently, the recessed portions 541B and 541C are directed to the depressed portions 1402′ and 1402″ with a higher degree of accuracy, respectively.

Also with such an eighth embodiment as described above, effects similar to those of the aforementioned first embodiment can be demonstrated.

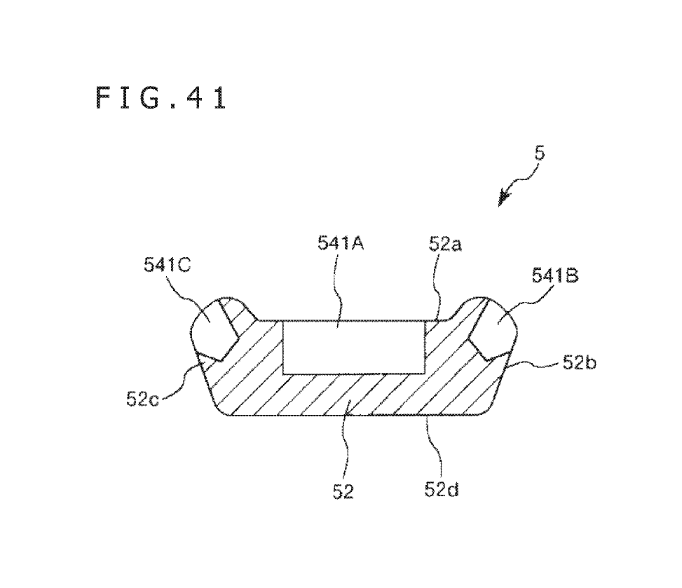

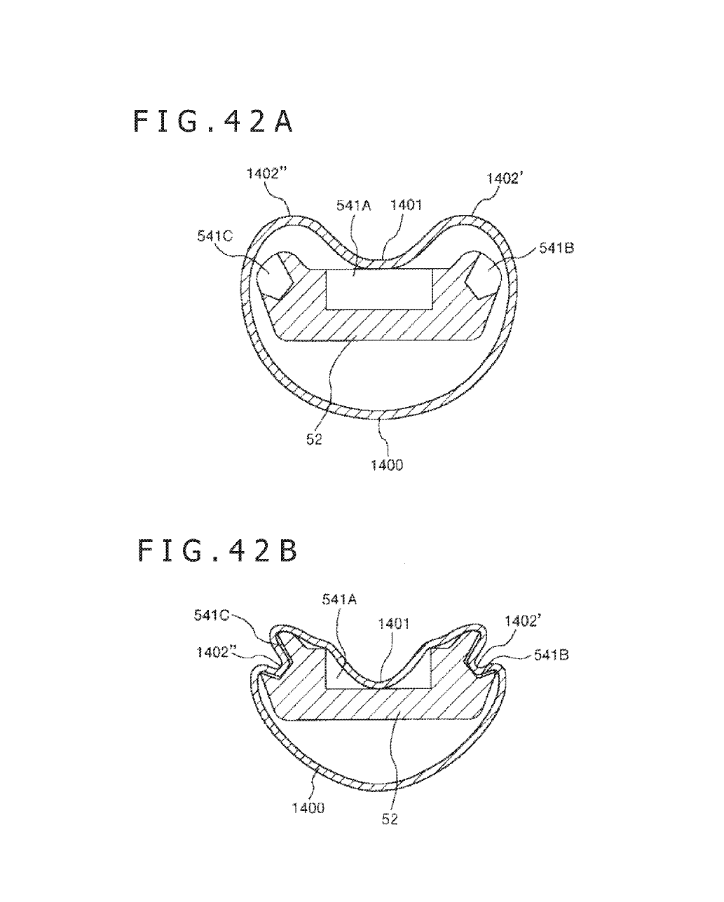

FIG. 41 is a sectional view depicting a distal portion of a vaginal-insertion tool, which includes a puncture apparatus according to a ninth embodiment. FIGS. 42A and 42B depict the distal portion depicted in FIG. 41 in a state in which it is inserted in the vagina, and wherein FIG. 42A is a view depicting the distal portion before the vaginal wall is absorbed and FIG. 42B is a view depicting the distal portion in a state in which the vaginal wall is absorbed. Note that FIG. 41 is a sectional view corresponding to FIG. 39B.

Although the ninth embodiment of the puncture apparatus is described below with reference to FIGS. 41 to 42B, the description is given principally of differences of the ninth embodiment from the aforementioned embodiments while description of like matters are omitted herein to avoid redundancy.

The present embodiment is similar to the aforementioned eighth embodiment except that it is different in configuration of the vaginal-insertion tool, particularly in regard to the shape of the distal portion.

As depicted in FIG. 41, the vaginal-insertion tool 5 of the present embodiment has a substantially trapezoidal transverse sectional shape, and the upper face 52a has a shape recessed to the inner side (lower face 52d side). Since the upper face 52a has a recessed shape in this manner, the distal portion 52 has such a shape that the suction holes 54B and 54C open to the side faces 52b and 52c project to the outer periphery side of the distal portion 52 farther than the suction hole 54A open to the upper face 52a.

Consequently, when the distal portion 52 is inserted in the vagina 1400 as depicted in FIG. 42A, the recessed portions 541B and 541C can be disposed closely to the depressed portions 1402′ and 1402″. Further, upon absorption, the depressed portions 1402′ and 1402″ can be absorbed to the recessed portions 541B and 541C with a relative higher degree of certainty as depicted in FIG. 42B.

Also with such a ninth embodiment as described above, effects similar to those of the aforementioned eighth embodiment can be demonstrated.

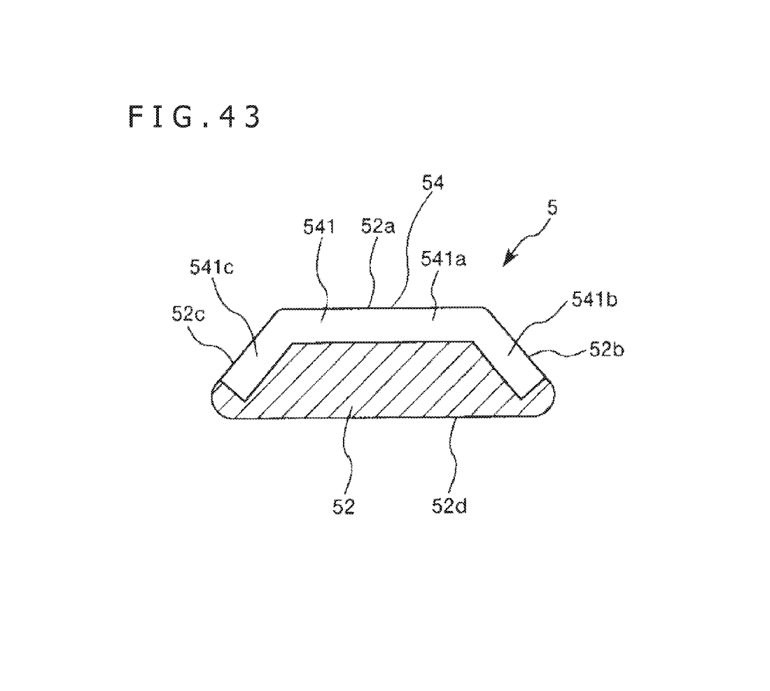

FIG. 43 is a sectional view depicting a distal portion of a vaginal-insertion tool, which includes a puncture apparatus according to a tenth embodiment. Note that FIG. 43 is a sectional view corresponding to FIG. 39B.

Although the tenth embodiment of the puncture apparatus is described below with reference to FIG. 43, the description is given principally of differences of the tenth embodiment from the aforementioned embodiments while description of like matters are omitted herein to avoid redundancy.

The present embodiment is similar to the aforementioned eighth embodiment except that it is different in configuration of the vaginal-insertion tool.

As depicted in FIG. 43, in the vaginal-insertion tool 5 of the present embodiment, a suction hole 54 having a recessed portion 541 open over the upper face 52a and the side faces 52b and 52c is provided. In such a suction hole 54 as just described, suction directions are different from each other among a portion 541a open to the upper face 52a of the recessed portion 541, another portion 541b open to the side face 52b, and a further portion 541c open to the side face 52c.

Also with such a tenth embodiment as described above, effects similar to those of the aforementioned eighth embodiment can be demonstrated.

Note that, while the upper face 52a and the side faces 52b and 52c in the present embodiment are individually configured from a flat face and the side faces 52b and 52c are inclined with respect to the upper face 52a, the configuration of the distal portion 52 is not limited to this. For example, the upper face 52a and the side faces 52b and 52c may be configured from curved faces continuing to each other. With such a shape as just described, effects similar to those of the present embodiment can be demonstrated.

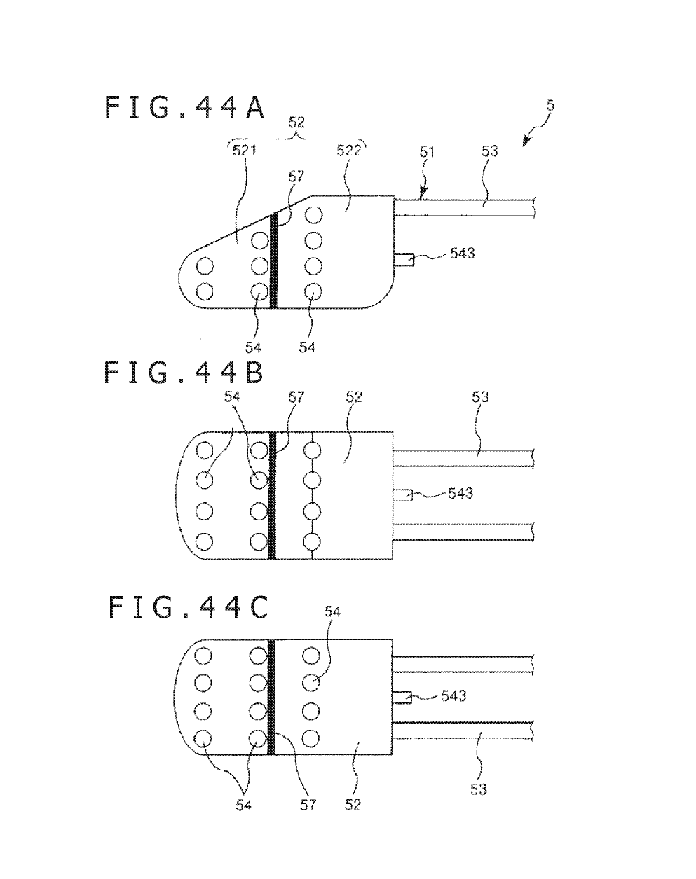

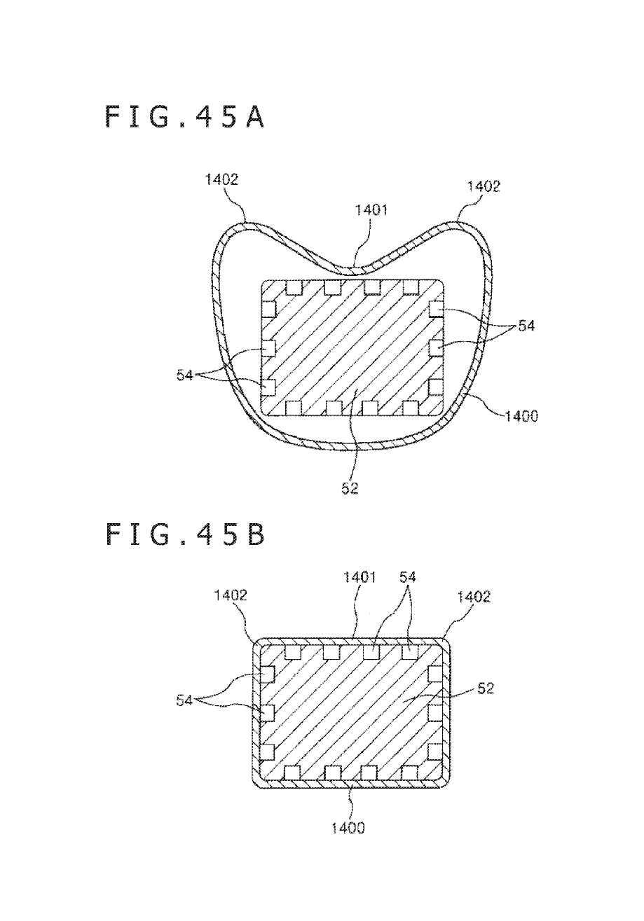

FIGS. 44A to 44C depict a distal portion of a vaginal-insertion tool, which includes a puncture apparatus according to an eleventh embodiment, and wherein FIG. 44A is a lateral view, FIG. 44B is a top plan view, and FIG. 44C is a bottom plan view. FIGS. 45A and 45B depict the distal portion depicted in FIGS. 44A to 44C in a state in which it is inserted in the vagina, and wherein FIG. 45A is a view depicting the distal portion in a state before the vaginal wall is absorbed and FIG. 45B is a view depicting the distal portion in another state in which the vaginal wall is absorbed.

Although the eleventh embodiment of the puncture apparatus is described below with reference to FIGS. 44A to 45B, the description is given principally of differences of the eleventh embodiment from the aforementioned embodiments while description of like matters are omitted herein to avoid redundancy.

The present embodiment is similar to the aforementioned first embodiment except that it is different in configuration of the vaginal-insertion tool.

As depicted in FIGS. 44A to 44C, in the vaginal-insertion tool 5 of the present embodiment, a plurality of suction holes 54 are provided over an overall region in a circumferential direction of the tapering portion 521 of the distal portion 52. Consequently, as depicted in FIG. 45B, not only the vaginal anterior wall but also the overall periphery of the vaginal wall can be absorbed. Therefore, the vaginal wall can be absorbed with a relative higher degree of effectiveness to the distal portion 52.

Particularly, in the present embodiment, a plurality of suction holes 54 are provided along the circumferential direction of the distal portion 52 and a plurality of suction holes 54 are provided along the longitudinal direction of the distal portion 52. Thus, the suction holes 54 are disposed over the overall region of the tapering portion 521. Consequently, since the suction hole 54 can be disposed in a wider range with the size of the suction holes 54 reduced to a suitable size, the vaginal wall can be absorbed with a relative higher degree of effectiveness while preventing damage to the vaginal wall upon absorption.

Further, since the suction holes 54 in the present embodiment are provided at least in and around a region positioned on the plane (plane f9) of the distal portion 52 including the puncture route of the puncture member 3, the vaginal anterior wall in the proximity of the puncture route of the puncture member 3 can be absorbed to the distal portion 52 with a relative higher degree of certainty. Therefore, a state in which the urethral wall and the vaginal anterior wall are sufficiently spaced away from each other in order to perform puncture by the puncture member 3 can be established with a relative higher degree of certainty.

Note that, while the suction holes 54 in the present embodiment have a substantially circular opening shape, the opening shape of the suction holes 54 is not restricted particularly to this. For example, a longitudinal shape extending in the peripheral direction of the distal portion 52 may be applied or a longitudinal shape extending in the longitudinal direction of the distal portion 52 may be applied. Further, for example, the suction holes 54 may have opening shapes whose shapes or sizes are different from each other.

Since, in the present embodiment, the inflow reduction portion 522 is provided at the proximal side of the tapering portion 521 at which the suction holes 54 are provided, the aforesaid effects can be demonstrated more remarkably together with the effects of the inflow reduction portion 522.

Also with such an eleventh embodiment as described above, effects similar to those of the aforementioned first embodiment can be demonstrated.

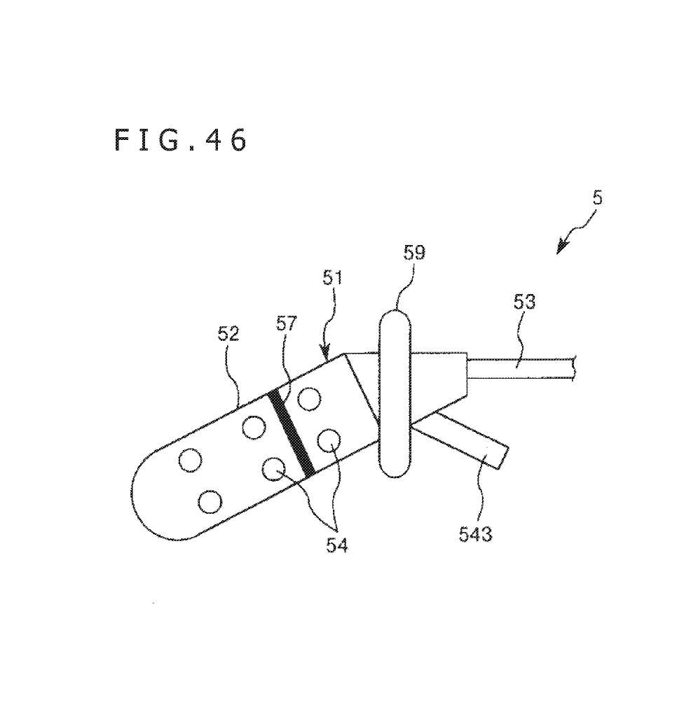

FIG. 46 is a view depicting a distal portion of a vaginal-insertion tool, which includes a puncture apparatus according to a twelfth embodiment.

Although the twelfth embodiment of the puncture apparatus is described below with reference to FIG. 46, the description is given principally of differences of the twelfth embodiment from the aforementioned embodiments while description of like matters are omitted herein to avoid redundancy.

The present embodiment is similar to the aforementioned eleventh embodiment except that it is different in configuration of the vaginal-insertion tool.

As depicted in FIG. 46, in the vaginal-insertion tool 5 of the present embodiment, the distal portion 52 has a flattened shape crushed in the heightwise direction. Also in such a distal portion 52 as just described, a plurality of suction holes 54 are provided in an overall region thereof in a circumferential direction similarly as in the aforementioned eleventh embodiment. Further, the inflow reduction portion 59 is provided on the proximal side of a region in which the suction hole 54 of the distal portion 52 is formed, such that is projects from the distal portion 52.

Also with such a twelfth embodiment as described above, effects similar to those of the aforementioned eleventh embodiment can be demonstrated.

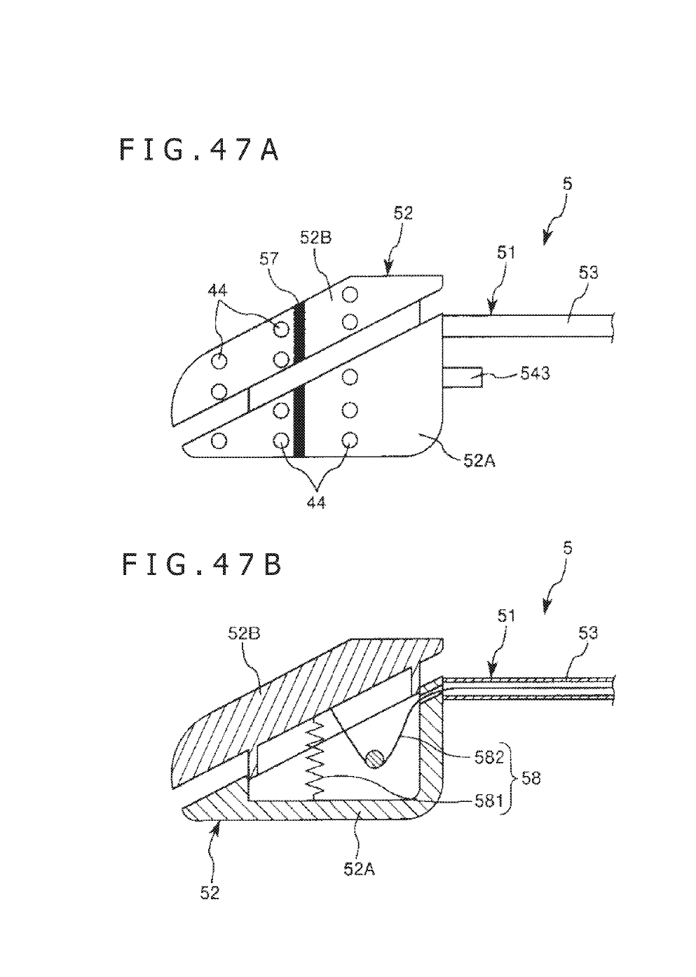

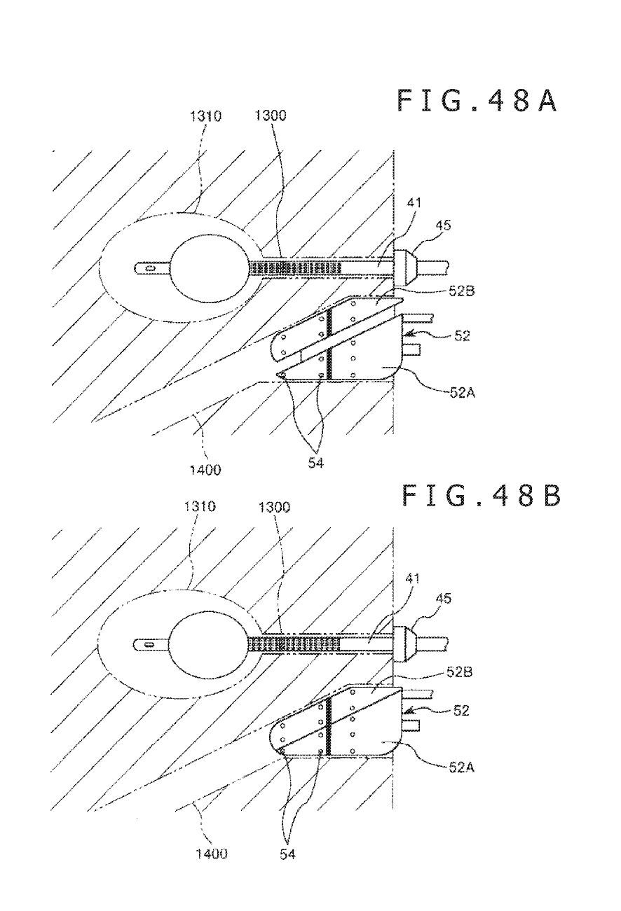

FIGS. 47A and 47B depict a distal portion of a vaginal-insertion tool which a puncture apparatus according to a thirteenth embodiment has, and wherein FIG. 47A is a lateral view and FIG. 47B is a sectional view. FIGS. 48A and 48B are views illustrating a function of the distal portion depicted in FIGS. 47A and 47B, respectively.

Although the thirteenth embodiment of the puncture apparatus is described below with reference to FIGS. 47A to 48B, the description is given principally of differences of the thirteenth embodiment from the aforementioned embodiments while description of like matters are omitted herein to avoid redundancy.

The present embodiment is similar to the aforementioned eleventh embodiment except that the distal portion of the vaginal-insertion tool is configured for deformation.

As depicted in FIG. 47A, in the vaginal-insertion tool 5 of the present embodiment, the distal portion 52 is configured from two divisional portions including a proximal part 52A and a movable part 52B. Further, the proximal part 52A is interlocked to the shaft portion 53 and the movable part 52B can be displaced upwardly and downwardly (juxtaposition direction of the urethral-insertion portion 41 and the vaginal-insertion portion 51) with respect to the proximal part 52A.

Although a displacement portion 58 for causing the movable part 52B to displace with respect to the proximal part 52A is not limited specifically, for example, as depicted in FIG. 47B, it can be configured such that it can include a spring (biasing portion) 581 for biasing the movable part 52B to the upper side (in a spacing direction) with respect to the proximal part 52A and a cord (operation portion) 582 interlocked to the movable part 52B and hooked on the proximal part 52A. Preferably, the cord 582 is pulled out to the proximity of the supporting portion 50 through the shaft portion 53 so as to be easily held by an operator. In the displacement portion 58 having such a configuration as just described, by pulling the cord 582, the movable part 52B can be displaced to the proximal part 52A side against the biasing force of the spring 581. After the displacement of the movable part 52B, the thickness of the distal portion 52 is small in comparison with that before the displacement.

The distal portion 52 having such a configuration as described above is used, for example, in such a manner as described below. First, as depicted in FIG. 48A, the distal portion 52 is inserted into the vagina 1400. Then, the vaginal wall is absorbed by the suction holes 54. Then, the cord 582 is pulled such that the movable part 52B is displaced downwardly as depicted in FIG. 48B. At this time, since the vaginal anterior wall absorbed by the movable part 52B is displaced to the lower side together with the movable part 52B, the space between the vagina 1400 and the urethra 1300 increases. Consequently, a puncture space for the puncture member 3 can be secured with a further increased size and puncture by the puncture member 3 can be performed with a relative higher degree of safety.

In accordance with an exemplary embodiment, for example, since the movable part 52B in the present embodiment is pushed into contact with the vaginal anterior wall by the spring 581, the movable part 52B can be closely contacted with the vaginal anterior wall and absorption of the vaginal anterior wall to the movable part 52B can be performed with a relative higher degree of certainty.

Also with such a thirteenth embodiment as described above, effects similar to those of the aforementioned eighth embodiment can be demonstrated.

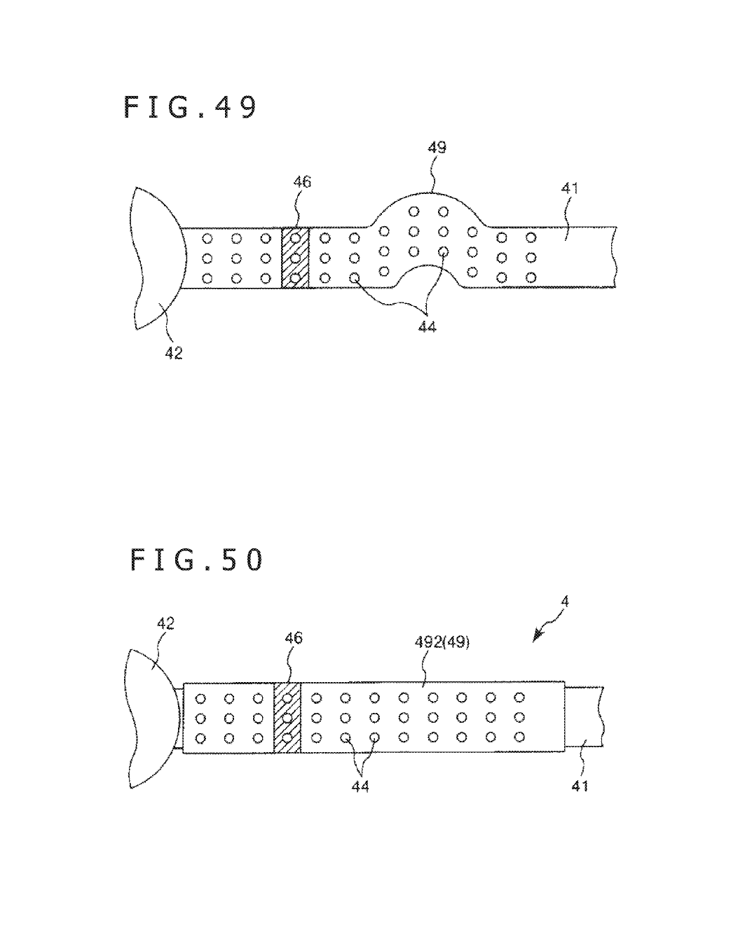

FIG. 49 is a lateral view depicting a urethral-insertion tool, which includes a puncture apparatus according to a fourteenth embodiment.

Although the fourteenth embodiment of the puncture apparatus is described below with reference to FIG. 49, the description is given principally of differences of the fourteenth embodiment from the aforementioned embodiments while description of like matters are omitted herein to avoid redundancy.

The present embodiment is similar to the aforementioned first embodiment except that it is different in configuration of the vaginal-insertion tool.

As depicted in FIG. 49, in the urethral-insertion tool 4 of the present embodiment, an intermediate portion (portion to be positioned in the urethra in the mounted state) of the urethral-insertion portion 41 is curved in a projection shape, and the portion curved in the projection shape configures the sliding resistance amplification portion 49.

Also with such a fourteenth embodiment as described above, effects similar to those of the aforementioned first embodiment can be demonstrated.

As a modification to the present embodiment, for example, an intermediate portion (portion to be positioned in the urethra in the mounted state) of the urethral-insertion portion 41 is curved by a plural number of times in a waveform such that the curved portion may configure a sliding resistance amplification portion 49.

FIG. 50 is a lateral view depicting a urethral-insertion tool, which includes a puncture apparatus according to a fifteenth embodiment.

Although the fifteenth embodiment of the puncture apparatus is described below with reference to FIG. 50, the description is given principally of differences of the fifteenth embodiment from the aforementioned embodiments while description of like matters are omitted herein to avoid redundancy.

The present embodiment is similar to the aforementioned first embodiment except that it is different in configuration of the vaginal-insertion tool.

As depicted in FIG. 50, in the urethral-insertion tool 4 of the present embodiment, a covering layer 492 for covering the urethral-insertion portion 41 is provided at an intermediate portion (portion to be positioned in the urethra in the mounted state) of the urethral-insertion portion 41. The covering layer 492 has sliding resistance with respect to the urethra higher than that of the surface of the urethral-insertion portion 41, and the sliding resistance amplification portion 49 is configured from the covering layer 492. Note that, in the present embodiment, since the covering layer 492 is provided in an overlapping relationship with the region S1, the suction holes 44 are formed such that they extend through the covering layer 492.

The constituent material for the covering layer 492 is not restricted particularly, and a material having a high friction coefficient such as various rubber materials such as, for example, natural rubber, acrylic rubber, styrene-butadiene rubber, urethane rubber, or silicone rubber can be used.

Also with such a fifteenth embodiment as described above, effects similar to those of the aforementioned first embodiment can be demonstrated.

Note that, while a step arising from the thickness of the covering layer 492 appears on the boundary between the covering layer 492 and the urethral-insertion portion 41 in the present embodiment, in order to eliminate the step, namely, in order to make the outer peripheral faces of the urethral-insertion portion 41 and the covering layer 492 flush with each other, a recessed portion corresponding to the thickness of the covering layer 492 may be formed in advance in a region in which the covering layer 492 of the urethral-insertion portion 41 is to be provided, which helps make it possible to perform insertion of the urethral-insertion portion 41 into the urethra with a relative higher degree of smoothness.

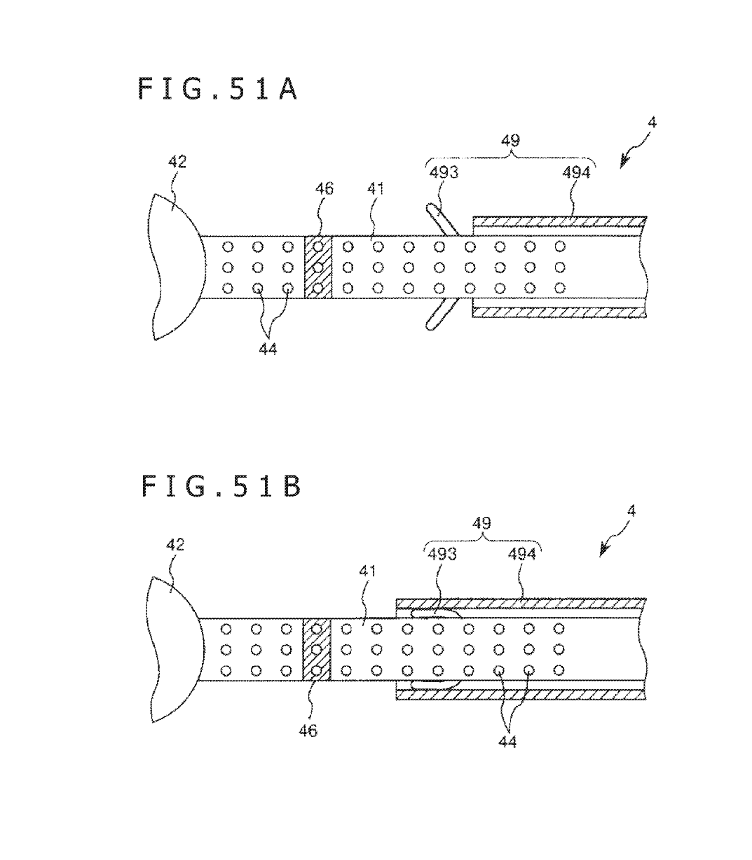

FIGS. 51A and 51B are partial sectional views depicting a urethral-insertion portion, which includes a puncture apparatus according to a sixteenth embodiment.

Although the sixteenth embodiment of the puncture apparatus is described below with reference to FIGS. 51A and 51B, the description is given principally of differences of the sixteenth embodiment from the aforementioned embodiments while description of like matters are omitted herein to avoid redundancy.

The present embodiment is similar to the aforementioned first embodiment except that it is different in configuration of the urethral-insertion tool.

As depicted in FIG. 51A, the sliding resistance amplification portion 49 of the present embodiment provided on the urethral-insertion tool 4 has projections (projection portions) 493 provided at an intermediate portion (portion to be positioned in the urethra in the mounted state) of the urethral-insertion portion 41 and projecting from the urethral-insertion portion 41. The projections 493 are inclined to the distal side and function as “barbs.”

Further, a cylindrical slider (cover member) 494 is provided on the outer periphery of the urethral-insertion portion 41 such that it is slidably movable in an axial direction with respect to the urethral-insertion portion 41. The slider 494 can move to a state for covering the projections 493 and another state for exposing the projections 493. The projections 493 are placed in a fallen state in the state thereof for covering the projection 493.

The projections 493 are configured for elastic deformation and configured from various rubber materials such as, for example, natural rubber, acrylic rubber, styrene-butadiene rubber, urethane rubber, or silicone rubber.

The urethral-insertion tool 4 having such a configuration as described above is placed in a state in which the projections 493 are covered with the slider 494 as depicted in FIG. 51B until the urethral-insertion portion 41 is inserted into the urethra. Then, after the urethral-insertion portion 41 is inserted into the urethra and positioned using the marker 45, the slider 494 is slidably moved to the proximal side to expose the projection 493 as depicted in FIG. 51A, and in this state, the urethral-insertion tool 4 is used.

With such usage as described above, since the urethral-insertion portion 41 is inserted into the urethra in a state in which the function of the sliding resistance amplification portion 49 is not demonstrated, the insertion can be performed smoothly. On the other hand, since the function of the sliding resistance amplification portion 49 is demonstrated after the urethral-insertion portion 41 is pushed into the inside of the body in a state in which the urethral-insertion portion 41 is sucked and fixed to the urethral wall, the urethra and the bladder can be pushed in with certainty together with the pushing.

In accordance with an exemplary embodiment, for example, since the projections 493 in the present embodiment function as “barbs,” the sliding resistance upon pushing in can be increased further.

Also with such a sixteenth embodiment as described above, effects similar to those of the aforementioned eighth embodiment can be demonstrated.

Note that, while two projections 493 are provided in the present embodiment, the number of projections 493 is not restricted particularly. Further, while the sliding resistance amplification portion 49 in the present embodiment has the projections (projection portions) 493, the configuration of it is not limited to this and at least one or more ones of projections or recesses may be provided on the sliding resistance amplification portion 49.

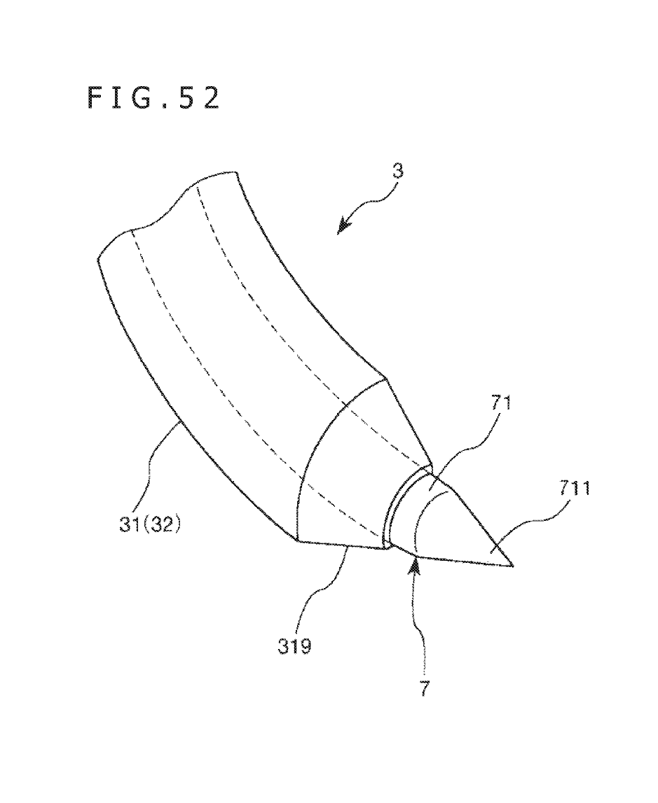

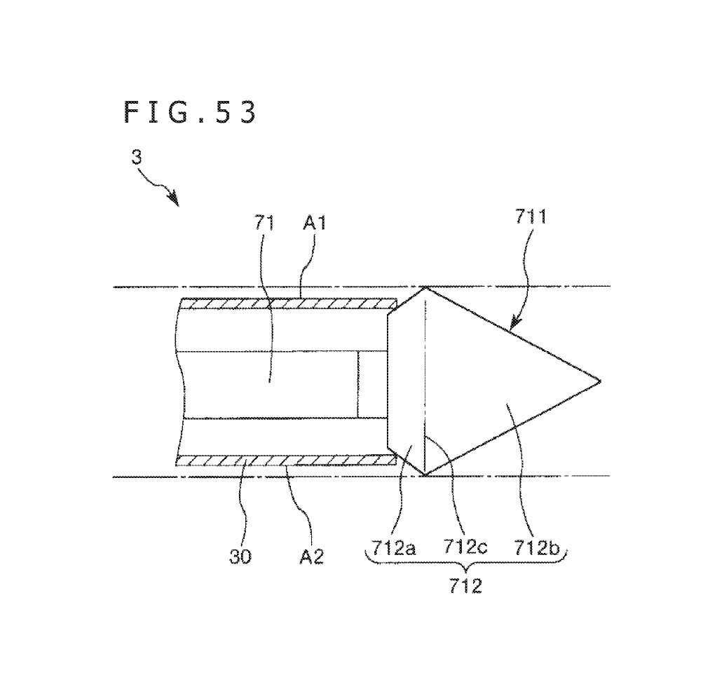

FIG. 52 is a perspective view depicting a puncture member, which includes a puncture apparatus according to a seventeenth embodiment. FIG. 53 is a sectional view depicting a modification to the puncture member depicted in FIG. 52.

Although the seventeenth embodiment of the puncture apparatus is described below with reference to FIGS. 52 and 53, the description is given principally of differences of the seventeenth embodiment from the aforementioned embodiments while description of like matters are omitted herein to avoid redundancy.

The present embodiment is similar to the aforementioned first embodiment except that it is different in configuration of the puncture member.

As depicted in FIG. 52, the puncture member 3 in the present embodiment is configured from a main body 31. In particular, for example, the puncture member 3 is configured by omitting the needle body 35 from the puncture member 3 in the aforementioned first embodiment. Further, in a state (initial state) in which the insertion portion 71 is inserted in the puncture member 3, the distal portion 711 which is a distal portion of the insertion portion 71 projects from the distal side opening of the main body 31. The distal portion 711 projecting from the main body 31 can also function as a needle tip of the puncture member 3.

Since the distal portion 711 of the insertion portion 71 functions also as the needle body of the puncture member 3, it is possible to contrive reduction of the number of members, for example, in comparison with the aforementioned first embodiment. Further, if the puncture member 3 is operated to puncture a living body and the insertion portion 71 is pulled out of the puncture member 3, then the distal side opening of the main body 31 can be opened. In other words, since it is unnecessary, in the present embodiment, to remove the needle body 35 in order to open the distal side opening of the main body 31 as in the aforementioned first embodiment, the operation can be carried out smoothly. Further, since the outer diameter of the insertion portion 71 and the inner diameter of the distal side opening of the main body 31 are set substantially equal to each other, displacement of the insertion portion 71 from the main body 31 can be prevented and the operability can thereby be enhanced.

In accordance with an exemplary embodiment, the main body 31 can have, at the distal portion thereof, a tapering portion 319 whose outer diameter gradually increases in a direction toward the proximal end from its distal side opening. The tapering portion 319 functions as a dissecting portion, which, as the distal portion 711 of the insertion portion 71 punctures a living body, dissects the living body in such a manner that it gradually expands the living body following the distal portion 711.

Note that, although a tapering angle of the tapering portion 319 and a tapering angle of the distal portion 711 may be equal to each other, preferably they are different from each other. In this case, it is preferable that the tapering angle of the tapering portion 319 is smaller than the tapering angle of the distal portion 711. This configuration can help enable a relative smooth puncture.

Also with such a seventeenth embodiment as described above, effects similar to those of the aforementioned first embodiment can be demonstrated.

As a modification to the present embodiment, the following configuration is available. As depicted in FIG. 53, the puncture member 3 is configured from a sheath 30. In particular, the puncture member 3 is configured by omitting the needle body 35 from the puncture member 3 in the aforementioned first embodiment. Further, in a state (initial state) in which the insertion portion 71 is inserted in the puncture member 3, the distal portion 711 which is a distal portion of the insertion portion 71 projects from the distal side opening of the main body 31.

The distal portion 711 is provided removable from the insertion portion 71 through screw engagement, fitting or the like. Further, the distal portion 711 has a needle tip 712 projecting from the distal end of the sheath 30. The needle tip 712 has a flattened shape following the sheath 30. Further, the needle tip 712 has a gradually increasing area portion 712a having a cross-sectional area gradually increasing toward the distal end, and a gradually decreasing area portion 712b provided on the distal side of the gradually increasing area portion 712a and having a cross-sectional area gradually decreasing toward the distal end. The minor axis of a boundary 712c between the gradually increasing area portion 712a and the gradually decreasing area portion 712b is longer than the minor axis of the distal end of the sheath 30, and the major axis of the boundary 712c is longer than the major axis of the distal end of the sheath 30, which helps make it possible to puncture the inside of a living body substantially only by the needle tip 712. Therefore, the puncture resistance can be reduced, and the living body can be punctured with a relative higher degree of smoothness. Note that the minor axis of the boundary 712c may be equal to the minor axis of the distal end of the sheath 30, and the major axis of the boundary 712c may be equal to the major axis of the distal end of the sheath 30.

Although the insertion tool and the insertion method for an insertion tool of the present disclosure have been described in connection with the embodiments depicted in the drawings, the present disclosure is not limited to them, and the components in the embodiments can be replaced by arbitrary members having similar functions. Further, some other arbitrary member or members may be added to the present disclosure. Further, the embodiments may be combined suitably.

Further, while a configuration in which the main body can be separated into a distal end divisional piece and a proximal end divisional piece is described in the aforementioned embodiments, the configuration of the main body is not limited to this, and a configuration that the main body cannot be separated into a distal end divisional piece and a proximal end divisional piece may be applied. In particular, the main body may be configured in the form of a single tube. In this case, also the state maintaining portion is omitted.

Further, while the sheath in one of the aforementioned embodiments is configured as part of the puncture member, the sheath is not limited to this. In particular, the puncture member may be used such that a sheath is inserted in a through-hole formed in the living body using some means. Describing the method given just above particularly in a corresponding relationship to the aforementioned first embodiment, the puncture apparatus 1 in which the puncture member 3 is omitted is prepared, and the distal portion 711 of the insertion portion 71 is caused to puncture, using the insertion portion 71 as the puncture member, the right side inguinal region of the patient. Then, the distal portion 711 is caused to pass one of the obturator foramens, the space between the urethra and the vagina, and the other one of the obturator foramens in order until the insertion portion is projected from the left side inguinal region to the outside of the body. Then, the insertion portion 71 is inserted into the inside of the puncture apparatus 1 and the sheath 30 (main body 31) is advanced to the inside of the body along the insertion portion 71 until a state in which the opposite ends thereof are projected from the body surface H is established. Then, the insertion portion 71 is removed from the inside of the body. Consequently, the sheath 30 is disposed into the living body. Then, the implant main body 91 is disposed into the sheath 30 and the sheath 30 is pulled off from within the body. The implant main body 91 can be indwelled thereby in the living body similarly as in the aforementioned embodiments.

Further, for example, the distal portion 711 of the insertion portion 71 is caused to puncture the right side inguinal region of the patient and then pass one of the obturator foramens, the space between the urethra and the vagina, and the other one of the obturator foramens in order until the distal portion 711 is projected from the left side inguinal region to the outside of the body. Thereafter, the distal portion of the sheath 30 is fixed to the distal portion 711. Then, the insertion portion 71 is rotated inversely and removed from the inside of the body so that the sheath 30 is indwelled in the living body. Then, if the implant main body 91 is disposed in the sheath 30 and then the sheath 30 is pulled out from within the body while the implant main body 91 is left in the living body, then the implant main body 91 can be indwelled in the living body similarly as in the aforementioned embodiments.

Further, in the aforementioned embodiments, a configuration is described that the implant main body is inserted into the main body after the main body of the puncture member is disposed into the living body, the embodiment is not limited to this, and the implant main body may be accommodated in the puncture member (main body) from the beginning. In this case, for example, it can be preferable to fix a string positioned on the needle tip side from between two strings, which the implant main body has to the needle tip in advance. This makes it possible to project, when the needle tip is removed from the main body, the string to the outside of the main body together with the removal of the needle tip. Therefore, fine adjustment of the disposition of the implant main body after the process just described or the like can be performed relatively smoothly.

Further, while a case in which the puncture apparatus is applied to a device for use in embedding in a living body an embeddable implant for treatment of female urinary incontinence has been described in the aforementioned embodiments, the application of the puncture apparatus is not limited to the described one.

For example, the target of the application of the present disclosure can include excretory disorders attendant on the weakening of the pelvic floor muscle group (urinary urgency, frequent urination, urinary incontinence, fecal incontinence, urinary retention, strangury and so forth) and pelvic floor disorders including pelvic organ prolapse, vesicovaginal fistula, urethrovaginal fistula, pelvic pain or the like. The pelvic organ prolapse includes disorders of cystocele, enterocele, rectocele, hysterocele and so forth. Alternatively, the pelvic organ prolapse includes such disorders as anterior vaginal prolapse, posterior vaginal prolapse, vaginal apical prolapse, vaginal vault prolapse and the like in which the naming method thereof is due to the prolapsed vaginal-wall part.

Further, the overactive tissues include the bladder, vagina, uterus, bowel and the like. The less active tissues include bones, muscles, fascias, ligaments and the like. In particular, in the case of pelvic floor disorders, the less active tissues include an obturator fascia, a coccygeus fascia, a cardinal ligament, an uterosacral ligament, a sacrospinous ligament and the like.

The procedure for interlocking an overactive tissue with a less active tissue in the pelvic floor disorder, includes retropubic sling surgery, transobturator sling surgery (transobturator tape (TOT) surgery), tension-free vaginal mesh (TVM) surgery, uterosacral ligament suspension (USLS) surgery, sacrospinous ligament fixation (SSLF) surgery, iliococcygeus fascia fixation surgery, coccygeus fascia fixation surgery and the like.

The insertion tool of the present disclosure includes a vaginal-insertion tool having a vaginal-insertion portion for being inserted into the vagina and a suction portion provided at the vaginal-insertion portion, a urethral-insertion tool having a urethral-insertion portion for being inserted into the urethra, a maintaining portion capable of maintaining a relative positional relationship between the urethral-insertion portion and the vaginal-insertion portion, and a displacement portion capable of causing a distal portion of the vaginal-insertion portion to approach the urethral-insertion portion rather than the relative positional relationship maintained by the maintaining portion. Therefore, since the distal portion of the vaginal-insertion portion can be caused to approach the urethral-insertion portion by the displacement portion, absorption of the vaginal wall by the vaginal-insertion portion can be performed with a higher degree of certainty and a sufficient space can be secured between the vagina and the urethra. In particular, by inserting the vaginal-insertion portion and the urethral-insertion portion into the vagina and the urethra, respectively, and moving the vaginal-insertion portion toward the urethral-insertion portion, the vaginal-insertion portion can be closely contacted with the vaginal wall. In this state, the vaginal wall can be absorbed with a higher degree of certainty to the vaginal-insertion portion. Then, after the vaginal wall is absorbed, the vagina can be separated sufficiently from the urethra by spacing the vaginal-insertion portion away from the urethra insertion portion and maintaining (fixing) the postures of them by the maintaining portion. Consequently, a space sufficient for puncture by the puncture member can be secured between the vagina and the urethra. Therefore, puncture by the puncture member can be performed with safety.

Meanwhile, the insertion method for an insertion tool of the present disclosure is an insertion method for an insertion tool having a vaginal-insertion tool including a vaginal-insertion portion for being inserted into the vagina and having a suction hole provided at the vaginal-insertion portion and a urethral-insertion tool including a urethral-insertion portion for being inserted into the urethra, including inserting the vaginal-insertion portion into the vagina and inserting the urethral-insertion portion into the urethra, displacing at least one of the vaginal-insertion portion and the urethral-insertion portion in a direction spaced away from the other one of the vaginal-insertion portion and the urethral-insertion portion in a state in which the vaginal wall is sucked by the suction hole, and maintaining a relative positional relationship after the displacement between the vaginal-insertion portion and the urethral-insertion portion. Therefore, the vaginal-insertion portion can be closely contacted with the vaginal wall by inserting the vaginal-insertion portion and the urethral-insertion portion into the vagina and the urethra, respectively, and causing the vaginal-insertion portion to approach the urethral-insertion portion. In this state, the vaginal wall can be absorbed with a higher degree of certainty to the vaginal-insertion portion. Then, after the vaginal wall is absorbed, the vagina can be separated sufficiently from the urethra by spacing the vaginal-insertion portion away from the urethra insertion portion and maintaining (fixing) the postures of them by the maintaining portion. Consequently, a space sufficient for puncture by the puncture member can be secured between the vagina and the urethra. Therefore, puncture by the puncture member can be performed with safety.

Accordingly, the insertion tool and the insertion method for an insertion tool of the present disclosure have industrial applicability.

The detailed description above describes an insertion tool and an insertion method for an insertion tool. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An insertion tool, comprising:
a vaginal-insertion tool having a vaginal-insertion portion configured to be inserted into a vagina and a suction portion provided at the vaginal-insertion portion;
a urethral-insertion tool having a urethral-insertion portion configured to be inserted into a urethra;
a maintaining portion capable of maintaining a relative positional relationship between the urethral-insertion portion and the vaginal-insertion portion; and
a displacement portion capable of causing a distal portion of the vaginal-insertion portion to approach the urethral-insertion portion rather than the relative positional relationship maintained by the maintaining portion.

2. The insertion tool according to claim 1, wherein the suction portion is provided at least in the vaginal-insertion portion on a side of the urethral-insertion portion.

3. The insertion tool according to claim 1,
wherein the vaginal-insertion tool has a vaginal-insertion portion supporting portion supporting the vaginal-insertion portion,
the urethral-insertion tool has a urethral-insertion portion supporting portion supporting the urethral-insertion portion, and
the displacement portion interlocks the vaginal-insertion portion supporting portion and the urethral-insertion portion supporting portion for rotary movement to each other.

4. The insertion tool according to claim 1,
wherein the vaginal-insertion tool has a vaginal-insertion portion supporting portion supporting the vaginal-insertion portion,
the urethral-insertion tool has a urethral-insertion portion supporting portion supporting the urethral-insertion portion, and
the displacement portion interlocks the vaginal-insertion portion supporting portion and the urethral-insertion portion supporting portion for movement toward and away from each other.

5. The insertion tool according to claim 3, wherein the maintaining portion includes a pawl portion provided at one of the vaginal-insertion portion supporting portion and the urethral-insertion portion supporting portion and an engagement portion provided at the other one of the vaginal-insertion portion supporting portion and the urethral-insertion portion supporting portion and configured to engage with the pawl portion.

6. The insertion tool according to claim 5, wherein the engagement between the pawl portion and the engagement portion can be cancelled.

7. The insertion tool according to claim 4, wherein the maintaining portion includes a pawl portion provided at one of the vaginal-insertion portion supporting portion and the urethral-insertion portion supporting portion and an engagement portion provided at the other one of the vaginal-insertion portion supporting portion and the urethral-insertion portion supporting portion and configured to engage with the pawl portion.

8. The insertion tool according to claim 7, wherein the engagement between the pawl portion and the engagement portion can be cancelled.

9. The insertion tool according to claim 1, wherein the displacement portion includes a hinge portion provided at an intermediate portion in a longitudinal direction of the vaginal-insertion portion for allowing the vaginal-insertion portion to be flexion-deformed at the intermediate portion in the longitudinal direction.

10. The insertion tool according to claim 9,
wherein the maintaining portion includes an operation portion disposed on an outer periphery of the vaginal-insertion portion and movable in the longitudinal direction with respect to the vaginal-insertion portion, and
the maintaining portion blocks the flexion-deformation by positioning the operation portion so as to overlap with the hinge portion but allows the flexion-deformation by positioning the operation portion so as to displace from the hinge portion.

11. An insertion method for an insertion tool having a vaginal-insertion tool including a vaginal-insertion portion configured to be inserted into a vagina and having a suction hole provided at the vaginal-insertion portion and a urethral-insertion tool including a urethral-insertion portion configured to be inserted into a urethra, the method comprising:
inserting the vaginal-insertion portion into the vagina and inserting the urethral-insertion portion into the urethra;
displacing at least one of the vaginal-insertion portion and the urethral-insertion portion in a direction spaced away from the other one of the vaginal-insertion portion and the urethral-insertion portion in a state in which a vaginal wall is sucked by the suction hole; and
maintaining a relative positional relationship between the vaginal-insertion portion and the urethral-insertion portion after the displacement.

12. The method according to claim 11, comprising:
providing the suction portion at least in the vaginal-insertion portion on a side of the urethral-insertion portion.

13. The method according to claim 11, wherein the vaginal-insertion tool has a vaginal-insertion portion supporting portion supporting the vaginal-insertion portion, and the urethral-insertion tool has a urethral-insertion portion supporting portion supporting the urethral-insertion portion; and
interlocking the vaginal-insertion portion supporting portion and the urethral-insertion portion supporting portion for rotary movement to each other with the displacement portion.

14. The method according to claim 11, wherein the vaginal-insertion tool has a vaginal-insertion portion supporting portion supporting the vaginal-insertion portion, and the urethral-insertion tool has a urethral-insertion portion supporting portion supporting the urethral-insertion portion; and
interlocking the vaginal-insertion portion supporting portion and the urethral-insertion portion supporting portion for movement toward and away from each other with the displacement portion.

15. The method according to claim 13, wherein the maintaining portion includes a pawl portion provided at one of the vaginal-insertion portion supporting portion and the urethral-insertion portion supporting portion and an engagement portion provided at the other one of the vaginal-insertion portion supporting portion and the urethral-insertion portion supporting portion and configured to engage with the pawl portion.

16. The method according to claim 15, comprising:
cancelling the engagement between the pawl portion and the engagement portion.

17. The method according to claim 14, wherein the maintaining portion includes a pawl portion provided at one of the vaginal-insertion portion supporting portion and the urethral-insertion portion supporting portion and an engagement portion provided at the other one of the vaginal-insertion portion supporting portion and the urethral-insertion portion supporting portion and configured to engage with the pawl portion.

18. The method according to claim 17, comprising:
cancelling the engagement between the pawl portion and the engagement portion.

19. The method according to claim 11, wherein the displacement portion includes a hinge portion provided at an intermediate portion in a longitudinal direction of the vaginal-insertion portion for allowing the vaginal-insertion portion to be flexion-deformed at the intermediate portion in the longitudinal direction.

20. The method according to claim 19, wherein the maintaining portion includes an operation portion disposed on an outer periphery of the vaginal-insertion portion and movable in the longitudinal direction with respect to the vaginal-insertion portion; and
blocking the flexion-deformation by positioning the operation portion so as to overlap with the hinge portion and allowing the flexion-deformation by positioning the operation portion so as to displace from the hinge portion.
